(19) United States
(12) Patent Application Publication
(10) Pub. No.: US 2010/0105134 A1
(43) Pub. Date: Apr. 29, 2010

(54) NUCLEIC ACID COMPOUNDS FOR INHIBITING GENE EXPRESSION AND USES THEREOF

(75) Inventors: Steven C. Quay, Seattle, WA (US); James McSwiggen, Boulder, CO (US); Narendra K. Vaish, Kirkland, WA (US); Mohammad Ahmadian, Carlsbad, CA (US)

Correspondence Address: SCHWABE, WILLIAMSON & WYATT, P.C., 1420 FIFTH, SUITE 3010, SEATTLE, WA, 98101, US

(73) Assignee: MDRNA, Inc., Bothell, WA (US)

(21) Appl. No.: 12/552,082

(22) Filed: Sep. 1, 2009

Related U.S. Application Data
(63) Continuation-in-part of application No. PCT/US2008/055371, filed on Feb. 28, 2008.
Application No. PCT/US2008/055371 is a continuation-in-part of application No. PCT/US2008/055370, filed on Feb. 28, 2008.
Application No. PCT/US2008/055370 is a continuation-in-part of application No. PCT/US2008/055362, filed on Feb. 28, 2008.
Application No. PCT/US2008/055362 is a continuation-in-part of application No. PCT/US2008/055380, filed on Feb. 28, 2008.
Application No. PCT/US2008/055380 is a continuation-in-part of application No. PCT/US2008/055383, filed on Feb. 28, 2008.
Application No. PCT/US2008/055383 is a continuation-in-part of application No. PCT/US2008/055375, filed on Feb. 28, 2008.
Application No. PCT/US2008/055375 is a continuation-in-part of application No. PCT/US2008/055360, filed on Feb. 28, 2008.
Application No. PCT/US2008/055360 is a continuation-in-part of application No. PCT/US2008/055374, filed on Feb. 28, 2008.
Application No. PCT/US2008/055374 is a continuation-in-part of application No. PCT/US2008/055357, filed on Feb. 28, 2008.
Application No. PCT/US2008/055357 is a continuation-in-part of application No. PCT/US2008/055381, filed on Feb. 28, 2008.
Application No. PCT/US2008/055381 is a continuation-in-part of application No. PCT/US2008/055378, filed on Feb. 28, 2008.
Application No. PCT/US2008/055378 is a continuation-in-part of application No. PCT/US2008/055372, filed on Feb. 28, 2008.
Application No. PCT/US2008/055372 is a continuation-in-part of application No. PCT/US2008/055345, filed on Feb. 28, 2008.
Application No. PCT/US2008/055345 is a continuation-in-part of application No. PCT/US2008/055377, filed on Feb. 28, 2008.
Application No. PCT/US2008/055377 is a continuation-in-part of application No. PCT/US2008/055376, filed on Feb. 28, 2008.
Application No. PCT/US2008/055376 is a continuation-in-part of application No. PCT/US2008/055373, filed on Feb. 28, 2008.
Application No. PCT/US2008/055373 is a continuation-in-part of application No. PCT/US2008/055385, filed on Feb. 28, 2008.
Application No. PCT/US2008/055385 is a continuation-in-part of application No. PCT/US2008/055386, filed on Feb. 28, 2008.
Application No. PCT/US2008/055386 is a continuation-in-part of application No. PCT/US2008/055382, filed on Feb. 28, 2008.
Application No. PCT/US2008/055382 is a continuation-in-part of application No. PCT/US2008/055333, filed on Feb. 28, 2008.
Application No. PCT/US2008/055333 is a continuation-in-part of application No. PCT/US2008/055341, filed on Feb. 28, 2008.
Application No. PCT/US2008/055341 is a continuation-in-part of application No. PCT/US2008/055350, filed on Feb. 28, 2008.
Application No. PCT/US2008/055350 is a continuation-in-part of application No. PCT/US2008/055356, filed on Feb. 28, 2008.
Application No. PCT/US2008/055356 is a continuation-in-part of application No. PCT/US2008/055366, filed on Feb. 28, 2008.
Application No. PCT/US2008/055366 is a continuation-in-part of application No. PCT/US2008/055339, filed on Feb. 28, 2008.
Application No. PCT/US2008/055339 is a continuation-in-part of application No. PCT/US2008/055365, filed on Feb. 28, 2008.
Application No. PCT/US2008/055365 is a continuation-in-part of application No. PCT/US2008/055340, filed on Feb. 28, 2008.
Application No. PCT/US2008/055340 is a continuation-in-part of application No. PCT/US2008/055505, filed on Feb. 29, 2008.
Application No. PCT/US2008/055505 is a continuation-in-part of application No. PCT/US2008/055556, filed on Feb. 29, 2008.
Application No. PCT/US2008/055556 is a continuation-in-part of application No. PCT/US2008/055515, filed on Feb. 29, 2008.
Application No. PCT/US2008/055515 is a continuation-in-part of application No. PCT/US2008/055599, filed on Mar. 3, 2008.
Application No. PCT/US2008/055599 is a continuation-in-part of application No. PCT/US2008/055601, filed on Mar. 3, 2008.
Application No. PCT/US2008/055601 is a continuation-in-part of application No. PCT/US2008/055603, filed on Mar. 3, 2008.
Application No. PCT/US2008/055603 is a continuation-in-part of application No. PCT/US2008/055606, filed on Mar. 3, 2008.
Application No. PCT/US2008/055606 is a continuation-in-part of application No. PCT/US2008/055548, filed on Feb. 29, 2008.
Application No. PCT/US2008/055548 is a continuation-in-part of application No. PCT/US2008/055611, filed on Mar. 3, 2008.
Application No. PCT/US2008/055611 is a continuation-in-part of application No. PCT/US2008/055615, filed on Mar. 3, 2008.
Application No. PCT/US2008/055615 is a continuation-in-part of application No. PCT/US2008/055709, filed on Mar. 3, 2008.
Application No. PCT/US2008/055709 is a continuation-in-part of application No. PCT/US2008/055618, filed on Mar. 3, 2008.
Application No. PCT/US2008/055618 is a continuation-in-part of application No. PCT/US2008/055644, filed on Mar. 3, 2008.
Application No. PCT/US2008/055644 is a continuation-in-part of application No. PCT/US2008/055651, filed on Mar. 3, 2008.
Application No. PCT/US2008/055651 is a continuation-in-part of application No. PCT/US2008/055649, filed on Mar. 3, 2008.
Application No. PCT/US2008/055649 is a continuation-in-part of application No. PCT/US2008/055711, filed on Mar. 3, 2008.
Application No. PCT/US2008/055711 is a continuation-in-part of application No. PCT/US2008/055635, filed on Mar. 3, 2008.
Application No. PCT/US2008/055635 is a continuation-in-part of application No. PCT/US2008/055524, filed on Feb. 29, 2008.
Application No. PCT/US2008/055524 is a continuation-in-part of application No. PCT/US2008/055572, filed on Mar. 3, 2008.
Application No. PCT/US2008/055572 is a continuation-in-part of application No. PCT/US2008/055627, filed on Mar. 3, 2008.
Application No. PCT/US2008/055627 is a continuation-in-part of application No. PCT/US2008/055697, filed on Mar. 3, 2008.
Application No. PCT/US2008/055697 is a continuation-in-part of application No. PCT/US2008/055662, filed on Mar. 3, 2008.
Application No. PCT/US2008/055662 is a continuation-in-part of application No. PCT/US2008/055678, filed on Mar. 3, 2008.
Application No. PCT/US2008/055678 is a continuation-in-part of application No. PCT/US2008/055368, filed on Feb. 2, 2008.
Application No. PCT/US2008/055368 is a continuation-in-part of application No. PCT/US2008/055676, filed on Mar. 3, 2008.
Application No. PCT/US2008/055676 is a continuation-in-part of application No. PCT/US2008/055550, filed on Feb. 29, 2008.
Application No. PCT/US2008/055550 is a continuation-in-part of application No. PCT/US2008/055560, filed on Feb. 29, 2008.
Application No. PCT/US2008/055560 is a continuation-in-part of application No. PCT/US2008/055698, filed on Mar. 3, 2008.
Application No. PCT/US2008/055698 is a continuation-in-part of application No. PCT/US2008/055695, filed on Mar. 3, 2008.
Application No. PCT/US2008/055695 is a continuation-in-part of application No. PCT/US2008/055701, filed on Mar. 3, 2008.
Application No. PCT/US2008/055701 is a continuation-in-part of application No. PCT/US2008/055693, filed on Mar. 3, 2008.
Application No. PCT/US2008/055693 is a continuation-in-part of application No. PCT/US2008/055704, filed on Mar. 3, 2008.
Application No. PCT/US2008/055704 is a continuation-in-part of application No. PCT/US2008/055708, filed on Mar. 3, 2008.
Application No. PCT/US2008/055708 is a continuation-in-part of application No. PCT/US2008/055597, filed on Mar. 3, 2008.
Application No. PCT/US2008/055597 is a continuation-in-part of application No. PCT/US2008/055604, filed on Mar. 3, 2008.
Application No. PCT/US2008/055604 is a continuation-in-part of application No. PCT/US2008/055608, filed on Mar. 3, 2008.
Application No. PCT/US2008/055608 is a continuation-in-part of application No. PCT/US2008/055353, filed on Mar. 3, 2008.
Application No. PCT/US2008/055353 is a continuation-in-part of application No. PCT/US2008/055631, filed on Mar. 3, 2008.
Application No. PCT/US2008/055631 is a continuation-in-part of application No. PCT/US2008/055563, filed on Mar. 3, 2008.
Application No. PCT/US2008/055563 is a continuation-in-part of application No. PCT/US2008/055612, filed on Mar. 3, 2008.
Application No. PCT/US2008/055612 is a continuation-in-part of application No. PCT/US2008/055622, filed on Mar. 3, 2008.
Application No. PCT/US2008/055622 is a continuation-in-part of application No. PCT/US2008/055625, filed on Mar. 3, 2008.
Application No. PCT/US2008/055625 is a continuation-in-part of application No. PCT/US2008/055527, filed on Feb. 29, 2008.
Application No. PCT/US2008/055527 is a continuation-in-part of application No. PCT/US2008/055533, filed on Feb. 29, 2008.
Application No. PCT/US2008/055533 is a continuation-in-part of application No. PCT/US2008/055554, filed on Feb. 29, 2008.
Application No. PCT/US2008/055554 is a continuation-in-part of application No. PCT/US2008/055511, filed on Feb. 29, 2008.
Application No. PCT/US2008/055511 is a continuation-in-part of application No. PCT/US2008/055532, filed on Feb. 29, 2008.
Application No. PCT/US2008/055532 is a continuation-in-part of application No. PCT/US2008/055516, filed on Feb. 29, 2008.
Application No. PCT/US2008/055516 is a continuation-in-part of application No. PCT/US2008/055551, filed on Feb. 29, 2008.
Application No. PCT/US2008/055551 is a continuation-in-part of application No. PCT/US2008/055519, filed on Feb. 29, 2008.
Application No. PCT/US2008/055519 is a continuation-in-part of application No. PCT/US2008/055542, filed on Feb. 29, 2008.
Application No. PCT/US2008/055542 is a continuation-in-part of application No. PCT/US2008/055526, filed on Feb. 29, 2008.
(60) Provisional application No. 60/934,930, filed on Mar. 16, 2007.
(60) Provisional application No. 60/934,940, filed on Mar. 2, 2007.
(60) Provisional application No. 60/934,930, filed on Mar. 16, 2007.
(60) Provisional application No. 60/934,940, filed on Mar. 2, 2007.
(60) Provisional application No. 60/934,941, filed on Apr. 13, 2007.
(60) Provisional application No. 60/934,932, filed on Apr. 20, 2007.
(60) Provisional application No. 60/934,930, filed on Mar. 16, 2007.
(60) Provisional application No. 60/934,940, filed on Mar. 2, 2007.
(60) Provisional application No. 60/934,931, filed on Apr. 20, 2007.
(60) Provisional application No. 60/934,934, filed on Apr. 24, 2007.
(60) Provisional application No. 60/934,928, filed on Apr. 24, 2007.
(60) Provisional application No. 60/934,943, filed on Apr. 25, 2007.
(60) Provisional application No. 60/934,942, filed on Apr. 25, 2007.
(60) Provisional application No. 60/934,930, filed on Mar. 16, 2007.
(60) Provisional application No. 60/934,940, filed on Mar. 2, 2007.
(60) Provisional application No. 60/934,931, filed on Apr. 20, 2007.
(60) Provisional application No. 60/934,934, filed on Apr. 24, 2007.
(60) Provisional application No. 60/934,928, filed on Apr. 24, 2007.
(60) Provisional application No. 60/934,943, filed on Apr. 25, 2007.
(60) Provisional application No. 60/934,965, filed on Jun. 18, 2007.
(60) Provisional application No. 60/934,929, filed on Jun. 14, 2007.
(60) Provisional application No. 60/934,954, filed on Jun. 6, 2007.
(60) Provisional application No. 60/934,940, filed on Mar. 2, 2007.
(60) Provisional application No. 60/934,930, filed on Mar. 16, 2007.
(60) Provisional application No. 60/934,930, filed on Mar. 16, 2007.
(60) Provisional application No. 60/934,940, filed on Mar. 2, 2007.
(60) Provisional application No. 60/934,921, filed on May 24, 2007.
(60) Provisional application No. 60/932,968, filed on May 30, 2007.
(60) Provisional application No. 60/932,969, filed on May 30, 2007.
(60) Provisional application No. 60/932,967, filed on Jun. 1, 2007.
(60) Provisional application No. 60/934,930, filed on Mar. 16, 2007.
(60) Provisional application No. 60/934,940, filed on Mar. 2, 2007.
(60) Provisional application No. 60/934,954, filed on Jun. 6, 2007.
(60) Provisional application No. 60/934,929, filed on Jun. 14, 2007.
(60) Provisional application No. 60/934,930, filed on Mar. 16, 2007.
(60) Provisional application No. 60/934,940, filed on Mar. 2, 2007.
(60) Provisional application No. 60/949,443, filed on Jul. 12, 2007.
(60) Provisional application No. 60/934,930, filed on Mar. 16, 2007.
(60) Provisional application No. 60/934,940, filed on Mar. 2, 2007.
(60) Provisional application No. 60/949,444, filed on Jul. 12, 2007.
(60) Provisional application No. 60/934,930, filed on Mar. 16, 2007.
(60) Provisional application No. 60/934,940, filed on Mar. 2, 2007.
(60) Provisional application No. 60/949,448, filed on Jul. 12, 2007.
(60) Provisional application No. 60/934,930, filed on Mar. 16, 2007.
(60) Provisional application No. 60/934,940, filed on Mar. 2, 2007.
(60) Provisional application No. 60/949,450, filed on Jul. 12, 2007.
(60) Provisional application No. 60/951,167, filed on Jul. 20, 2007.
(60) Provisional application No. 60/951,168, filed on Jul. 20, 2007.
(60) Provisional application No. 60/951,170, filed on Jul. 20, 2007.
(60) Provisional application No. 60/934,930, filed on Mar. 16, 2007.
(60) Provisional application No. 60/934,940, filed on Mar. 2, 2007.
(60) Provisional application No. 60/952,191, filed on Jul. 26, 2007.
(60) Provisional application No. 60/952,188, filed on Jul. 26, 2007.
(60) Provisional application No. 60/952,192, filed on Jul. 26, 2007.
(60) Provisional application No. 60/952,499, filed on Jul. 27, 2007.
(60) Provisional application No. 60/934,930, filed on Mar. 16, 2007.
(60) Provisional application No. 60/934,940, filed on Mar. 2, 2007.
(60) Provisional application No. 60/952,191, filed on Jul. 26, 2007.
(60) Provisional application No. 60/952,188, filed on Jul. 26, 2007.
(60) Provisional application No. 60/952,192, filed on Jul. 26, 2007.
(60) Provisional application No. 60/952,499, filed on Jul. 27, 2007.
(60) Provisional application No. 60/953,873, filed on Aug. 3, 2007.
(60) Provisional application No. 60/934,930, filed on Mar. 16, 2007.
(60) Provisional application No. 60/934,940, filed on Mar. 2, 2007.
(60) Provisional application No. 60/956,093, filed on Aug. 15, 2007.
(60) Provisional application No. 60/934,930, filed on Mar. 16, 2007.
(60) Provisional application No. 60/934,940, filed on Mar. 2, 2007.
(60) Provisional application No. 60/970,414, filed on Sep. 6, 2007.
(60) Provisional application No. 60/934,930, filed on Mar. 16, 2007.
(60) Provisional application No. 60/934,940, filed on Mar. 2, 2007.
(60) Provisional application No. 60/956,679, filed on Aug. 17, 2007.
(60) Provisional application No. 60/934,930, filed on Mar. 16, 2007.
(60) Provisional application No. 60/934,940, filed on Mar. 2, 2007.
(60) Provisional application No. 60/970,796, filed on Sep. 7, 2007.
(60) Provisional application No. 60/934,930, filed on Mar. 16, 2007.
(60) Provisional application No. 60/934,940, filed on Mar. 2, 2007.
(60) Provisional application No. 60/970,858, filed on Sep. 7, 2007.
(60) Provisional application No. 60/934,930, filed on Mar. 16, 2007.
(60) Provisional application No. 60/934,940, filed on Mar. 2, 2007.
(60) Provisional application No. 60/971,476, filed on Sep. 11, 2007.
(60) Provisional application No. 60/934,930, filed on Mar. 16, 2007.
(60) Provisional application No. 60/934,940, filed on Mar. 2, 2007.
(60) Provisional application No. 60/971,874, filed on Sep. 12, 2007.
(60) Provisional application No. 60/934,930, filed on Mar. 16, 2007.
(60) Provisional application No. 60/934,940, filed on Mar. 2, 2007.
(60) Provisional application No. 60/973,397, filed on Sep. 18, 2007.
(60) Provisional application No. 60/934,930, filed on Mar. 16, 2007.
(60) Provisional application No. 60/934,940, filed on Mar. 2, 2007.
(60) Provisional application No. 60/973,398, filed on Sep. 18, 2007.
(60) Provisional application No. 60/973,397, filed on Sep. 18, 2007.
(60) Provisional application No. 60/934,930, filed on Mar. 16, 2007.
(60) Provisional application No. 60/934,940, filed on Mar. 2, 2007.
(60) Provisional application No. 60/983,179, filed on Oct. 27, 2007.
(60) Provisional application No. 60/934,930, filed on Mar. 16, 2007.
(60) Provisional application No. 60/934,940, filed on Mar. 2, 2007.
(60) Provisional application No. 60/983,183, filed on Oct. 27, 2007.
(60) Provisional application No. 60/983,182, filed on Oct. 27, 2007.
(60) Provisional application No. 60/983,180, filed on Oct. 27, 2007.
(60) Provisional application No. 60/983,181, filed on Oct. 27, 2007.
(60) Provisional application No. 60/986,910, filed on Nov. 9, 2007.
(60) Provisional application No. 60/986,822, filed on Nov. 9, 2007.
(60) Provisional application No. 60/986,893, filed on Nov. 9, 2007.
(60) Provisional application No. 60/934,930, filed on Mar. 16, 2007.
(60) Provisional application No. 60/934,940, filed on Mar. 2, 2007.
(60) Provisional application No. 60/986,913, filed on Nov. 9, 2007.
(60) Provisional application No. 60/934,930, filed on Mar. 16, 2007.
(60) Provisional application No. 60/934,940, filed on Mar. 2, 2007.
(60) Provisional application No. 61/021,491, filed on Jan. 16, 2008.
(60) Provisional application No. 60/934,930, filed on Mar. 16, 2007.
(60) Provisional application No. 60/934,940, filed on Mar. 2, 2007.
(60) Provisional application No. 60/989,419, filed on Nov. 20, 2007.
(60) Provisional application No. 60/934,930, filed on Mar. 16, 2007.
(60) Provisional application No. 60/934,940, filed on Mar. 2, 2007.
(60) Provisional application No. 60/986,916, filed on Nov. 9, 2007.
(60) Provisional application No. 60/934,930, filed on Mar. 16, 2007.
(60) Provisional application No. 60/934,940, filed on Mar. 2, 2007.
(60) Provisional application No. 60/986,919, filed on Nov. 9, 2007.
(60) Provisional application No. 60/934,930, filed on Mar. 16, 2007.
(60) Provisional application No. 60/934,940, filed on Mar. 2, 2007.
(60) Provisional application No. 60/987,733, filed on Nov. 13, 2007.
(60) Provisional application No. 60/934,930, filed on Mar. 16, 2007.
(60) Provisional application No. 60/934,940, filed on Mar. 2, 2007.
(60) Provisional application No. 60/987,737, filed on Nov. 13, 2007.
(60) Provisional application No. 60/934,930, filed on Mar. 16, 2007.
(60) Provisional application No. 60/934,940, filed on Mar. 2, 2007.
(60) Provisional application No. 60/987,740, filed on Nov. 13, 2007.
(60) Provisional application No. 60/988,076, filed on Nov. 14, 2007.
(60) Provisional application No. 60/988,079, filed on Nov. 14, 2007.
(60) Provisional application No. 60/988,082, filed on Nov. 14, 2007.
(60) Provisional application No. 60/988,083, filed on Nov. 14, 2007.
(60) Provisional application No. 60/934,930, filed on Mar. 16, 2007.
(60) Provisional application No. 60/934,940, filed on Mar. 2, 2007.
(60) Provisional application No. 61/013,979, filed on Dec. 14, 2007.
(60) Provisional application No. 60/934,930, filed on Mar. 16, 2007.
(60) Provisional application No. 60/934,940, filed on Mar. 2, 2007.
(60) Provisional application No. 60/988,395, filed on Nov. 15, 2007.
(60) Provisional application No. 60/934,930, filed on Mar. 16, 2007.
(60) Provisional application No. 60/934,940, filed on Mar. 2, 2007.
(60) Provisional application No. 60/988,397, filed on Nov. 15, 2007.
(60) Provisional application No. 60/988,398, filed on Nov. 15, 2007.
(60) Provisional application No. 60/934,930, filed on Mar. 16, 2007.
(60) Provisional application No. 60/934,940, filed on Mar. 2, 2007.
(60) Provisional application No. 60/988,399, filed on Nov. 15, 2007.
(60) Provisional application No. 60/934,930, filed on Mar. 16, 2007.
(60) Provisional application No. 60/934,940, filed on Mar. 2, 2007.
(60) Provisional application No. 60/988,400, filed on Nov. 15, 2007.
(60) Provisional application No. 60/988,401, filed on Nov. 15, 2007.
(60) Provisional application No. 60/988,402, filed on Nov. 15, 2007.
(60) Provisional application No. 60/934,930, filed on Mar. 16, 2007.
(60) Provisional application No. 60/934,940, filed on Mar. 2, 2007.
(60) Provisional application No. 60/986,944, filed on Nov. 9, 2007.
(60) Provisional application No. 60/988,403, filed on Nov. 15, 2007.
(60) Provisional application No. 61/013,981, filed on Dec. 14, 2007.
(60) Provisional application No. 60/934,930, filed on Mar. 16, 2007.
(60) Provisional application No. 60/934,940, filed on Mar. 2, 2007.
(60) Provisional application No. 61/015,611, filed on Dec. 20, 2007.
(60) Provisional application No. 60/934,930, filed on Mar. 16, 2007.
(60) Provisional application No. 60/934,940, filed on Mar. 2, 2007.
(60) Provisional application No. 60/988,405, filed on Nov. 15, 2007.
(60) Provisional application No. 60/989,427, filed on Nov. 20, 2007.
(60) Provisional application No. 60/989,424, filed on Nov. 20, 2007.
(60) Provisional application No. 60/989,428, filed on Nov. 20, 2007.
(60) Provisional application No. 60/934,930, filed on Mar. 16, 2007.
(60) Provisional application No. 60/934,940, filed on Mar. 2, 2007.
(60) Provisional application No. 61/014,305, filed on Dec. 17, 2007.
(60) Provisional application No. 60/934,930, filed on Mar. 16, 2007.
(60) Provisional application No. 60/934,940, filed on Mar. 2, 2007.
(60) Provisional application No. 61/014,730, filed on Dec. 18, 2007.
(60) Provisional application No. 60/934,930, filed on Mar. 16, 2007.
(60) Provisional application No. 60/934,940, filed on Mar. 2, 2007.
(60) Provisional application No. 61/015,164, filed on Dec. 19, 2007.
(60) Provisional application No. 60/934,930, filed on Mar. 16, 2007.
(60) Provisional application No. 60/934,940, filed on Mar. 2, 2007.
(60) Provisional application No. 61/015,166, filed on Dec. 19, 2007.
(60) Provisional application No. 60/934,930, filed on Mar. 16, 2007.
(60) Provisional application No. 60/934,940, filed on Mar. 2, 2007.
(60) Provisional application No. 61/012,401, filed on Dec. 7, 2007.
(60) Provisional application No. 60/934,930, filed on Mar. 16, 2007.
(60) Provisional application No. 60/934,940, filed on Mar. 2, 2007.
(60) Provisional application No. 61/016,340, filed on Dec. 21, 2007.
(60) Provisional application No. 60/934,930, filed on Mar. 16, 2007.
(60) Provisional application No. 60/934,940, filed on Mar. 2, 2007.
(60) Provisional application No. 60/992,808, filed on Dec. 6, 2007.
(60) Provisional application No. 60/934,930, filed on Mar. 16, 2007.
(60) Provisional application No. 60/934,940, filed on Mar. 2, 2007.
(60) Provisional application No. 61/013,982, filed on Dec. 14, 2007.
(60) Provisional application No. 60/934,930, filed on Mar. 16, 2007.
(60) Provisional application No. 60/934,940, filed on Mar. 2, 2007.
(60) Provisional application No. 61/014,139, filed on Dec. 17, 2007.
(60) Provisional application No. 60/934,930, filed on Mar. 16, 2007.
(60) Provisional application No. 60/934,940, filed on Mar. 2, 2007.
(60) Provisional application No. 60/934,921, filed on May 24, 2007.
(60) Provisional application No. 60/932,968, filed on May 30, 2007.
(60) Provisional application No. 60/932,969, filed on May 30, 2007.
(60) Provisional application No. 60/932,967, filed on Jun. 1, 2007.
(60) Provisional application No. 60/934,930, filed on Mar. 16, 2007.
(60) Provisional application No. 60/934,940, filed on Mar. 2, 2007.
(60) Provisional application No. 60/934,921, filed on May 24, 2007.
(60) Provisional application No. 60/932,968, filed on May 30, 2007.
(60) Provisional application No. 60/932,969, filed on May 30, 2007.
(60) Provisional application No. 60/932,967, filed on Jun. 1, 2007.
(60) Provisional application No. 61/014,733, filed on Dec. 18, 2007.
(60) Provisional application No. 60/934,930, filed on Mar. 16, 2007.
(60) Provisional application No. 60/934,940, filed on Mar. 2, 2007.
(60) Provisional application No. 61/012,402, filed on Dec. 7, 2007.
(60) Provisional application No. 60/934,930, filed on Mar. 16, 2007.
(60) Provisional application No. 60/934,940, filed on Mar. 2, 2007.
(60) Provisional application No. 61/016,343, filed on Dec. 21, 2007.
(60) Provisional application No. 60/934,930, filed on Mar. 16, 2007.
(60) Provisional application No. 60/934,940, filed on Mar. 2, 2007.
(60) Provisional application No. 61/015,616, filed on Dec. 20, 2007.
(60) Provisional application No. 60/934,930, filed on Mar. 16, 2007.
(60) Provisional application No. 60/934,940, filed on Mar. 2, 2007.
(60) Provisional application No. 61/015,618, filed on Dec. 20, 2007.
(60) Provisional application No. 60/934,930, filed on Mar. 16, 2007.
(60) Provisional application No. 60/934,940, filed on Mar. 2, 2007.
(60) Provisional application No. 61/014,734, filed on Dec. 18, 2007.
(60) Provisional application No. 60/934,930, filed on Mar. 16, 2007.
(60) Provisional application No. 60/934,940, filed on Mar. 2, 2007.
(60) Provisional application No. 61/015,167, filed on Dec. 19, 2007.
(60) Provisional application No. 60/934,930, filed on Mar. 16, 2007.
(60) Provisional application No. 60/934,940, filed on Mar. 2, 2007.
(60) Provisional application No. 61/015,170, filed on Dec. 19, 2007.
(60) Provisional application No. 60/934,930, filed on Mar. 16, 2007.
(60) Provisional application No. 60/934,940, filed on Mar. 2, 2007.
(60) Provisional application No. 61/014,735, filed on Dec. 18, 2007.
(60) Provisional application No. 60/934,930, filed on Mar. 16, 2007.
(60) Provisional application No. 60/934,940, filed on Mar. 2, 2007.
(60) Provisional application No. 61/014,737, filed on Dec. 18, 2007.
(60) Provisional application No. 60/934,930, filed on Mar. 16, 2007.
(60) Provisional application No. 60/934,940, filed on Mar. 2, 2007.
(60) Provisional application No. 60/992,975, filed on Dec. 6, 2007.
(60) Provisional application No. 60/934,930, filed on Mar. 16, 2007.
(60) Provisional application No. 60/934,940, filed on Mar. 2, 2007.
(60) Provisional application No. 61/015,171, filed on Dec. 19, 2007.
(60) Provisional application No. 60/934,930, filed on Mar. 16, 2007.
(60) Provisional application No. 60/934,940, filed on Mar. 2, 2007.
(60) Provisional application No. 61/015,173, filed on Dec. 19, 2007.
(60) Provisional application No. 60/934,930, filed on Mar. 16, 2007.
(60) Provisional application No. 60/934,940, filed on Mar. 2, 2007.
(60) Provisional application No. 61/012,404, filed on Dec. 7, 2007.
(60) Provisional application No. 60/934,930, filed on Mar. 16, 2007.
(60) Provisional application No. 60/934,940, filed on Mar. 2, 2007.
(60) Provisional application No. 61/014,738, filed on Dec. 18, 2007.
(60) Provisional application No. 60/934,930, filed on Mar. 16, 2007.
(60) Provisional application No. 60/934,940, filed on Mar. 2, 2007.
(60) Provisional application No. 61/015,619, filed on Dec. 20, 2007.
(60) Provisional application No. 60/934,930, filed on Mar. 16, 2007.
(60) Provisional application No. 60/934,940, filed on Mar. 2, 2007.
(60) Provisional application No. 60/934,930, filed on Mar. 16, 2007.
(60) Provisional application No. 60/934,940, filed on Mar. 2, 2007.
(60) Provisional application No. 61/016,319, filed on Dec. 21, 2007.
(60) Provisional application No. 60/934,930, filed on Mar. 16, 2007.
(60) Provisional application No. 60/934,940, filed on Mar. 2, 2007.
(60) Provisional application No. 60/934,930, filed on Mar. 16, 2007.
(60) Provisional application No. 60/934,940, filed on Mar. 2, 2007.
(60) Provisional application No. 60/934,930, filed on Mar. 16, 2007.
(60) Provisional application No. 60/934,940, filed on Mar. 2, 2007.
(60) Provisional application No. 60/934,930, filed on Mar. 16, 2007.
(60) Provisional application No. 60/934,940, filed on Mar. 2, 2007.
(60) Provisional application No. 60/934,930, filed on Mar. 16, 2007.
(60) Provisional application No. 60/934,940, filed on Mar. 2, 2007.
(60) Provisional application No. 60/934,930, filed on Mar. 16, 2007.
(60) Provisional application No. 60/934,940, filed on Mar. 2, 2007.
(60) Provisional application No. 60/934,930, filed on Mar. 16, 2007.
(60) Provisional application No. 60/934,940, filed on Mar. 2, 2007.
(60) Provisional application No. 60/934,930, filed on Mar. 16, 2007.
(60) Provisional application No. 60/934,940, filed on Mar. 2, 2007.

(51) Int. Cl. C12N 5/071; C07H 21/02; C12N 5/00

(52) U.S. Cl. 435/366; 536/23.1; 435/375

(57) ABSTRACT

The present disclosure provides RNA molecules, for example, meroduplex ribonucleic acid molecules (mdRNA), and blunt ended double-stranded ribonucleic acid molecules capable of decreasing or silencing expression of a target gene. An mdRNA of this disclosure comprises at least three strands that combine to form at least two non-overlapping double-stranded regions separated by a nick or gap wherein one strand is complementary to a target mRNA. Also provided are methods of decreasing expression of a target gene in a cell or in a subject to treat a disease or condition associated with the target gene.

### CROSS-REFERENCE TO RELATED APPLICATIONS

This application is a continuation-in-part of:

(1) International Application No. PCT/US2008/055371, filed Feb. 28, 2008, which claims the benefit of U.S. Provisional Patent Application Nos. 60/934,930, filed Mar. 16, 2007, and 60/934,940, filed Mar. 2, 2007;

(2) International Application No. PCT/US2008/055370, filed Feb. 28, 2008, which claims the benefit of U.S. Provisional Patent Application Nos. 60/934,930, filed Mar. 16, 2007, 60/934,940, filed Mar. 2, 2007, 60/934,941, filed Apr. 13, 2007, and 60/934,932, filed Apr. 20, 2007;

(3) International Application No. PCT/US2008/055362, filed Feb. 28, 2008, which claims the benefit of U.S. Provisional Patent Application Nos. 60/934,930, filed Mar. 16, 2007, 60/934,940, filed Mar. 2, 2007, 60/934,931, filed Apr. 20, 2007, 60/934,934, filed Apr. 24, 2007, 60/934,928, filed Apr. 24, 2007, 60/934,943, filed Apr. 25, 2007, and 60/934,942, filed Apr. 25, 2007;

(4) International Application No. PCT/US2008/055380, filed Feb. 28, 2008, which claims the benefit of U.S. Provisional Patent Application Nos. 60/934,930, filed Mar. 16, 2007, 60/934,940, filed Mar. 2, 2007, 60/934,931, filed Apr. 20, 2007, 60/934,934, filed Apr. 24, 2007, 60/934,928, filed Apr. 24, 2007, 60/934,943, filed Apr. 25, 2007, and 60/934,949, filed May 3, 2007;

(5) International Application No. PCT/US2008/055383, filed Feb. 28, 2008, which claims the benefit of U.S. Provisional Patent Application Nos. 60/934,930, filed Mar. 16, 2007, 60/934,940, filed Mar. 2, 2007, 60/934,941, filed Apr. 13, 2007, 60/934,932, filed Apr. 20, 2007, 60/934,933, filed Apr. 20, 2007, and 60/934,944, filed May 10, 2007;

(6) International Application No. PCT/US2008/055375, filed Feb. 28, 2008, which claims the benefit of U.S. Provisional Patent Application Nos. 60/934,930, filed Mar. 16, 2007, 60/934,940, filed Mar. 2, 2007, 60/934,946, filed May 3, 2007, 60/934,945, filed May 10, 2007, 60/934,935, filed May 15, 2007, and 60/934,922, filed May 17, 2007;

(7) International Application No. PCT/US2008/055360, filed Feb. 28, 2008, which claims the benefit of U.S. Provisional Patent Application Nos. 60/934,930, filed Mar. 16, 2007, 60/934,940, filed Mar. 2, 2007, 60/934,946, filed May 3, 2007, 60/934,945, filed May 10, 2007, 60/934,935, filed May 15, 2007, 60/934,922, filed May 17, 2007, and 60/932,970, filed May 22, 2007;

(8) International Application No. PCT/US2008/055374, filed Feb. 28, 2008, which claims the benefit of U.S. Provisional Patent Application Nos. 60/934,930, filed Mar. 16, 2007, 60/934,940, filed Mar. 2, 2007, 60/934,921, filed May 24, 2007, 60/932,968, filed May 30, 2007, 60/932,969, filed May 30, 2007, and 60/932,967, filed Jun. 1, 2007;

(9) International Application No. PCT/US2008/055381, filed Feb. 28, 2008, which claims the benefit of U.S. Provisional Patent Application Nos. 60/934,930, filed Mar. 16, 2007, 60/934,940, filed Mar. 2, 2007, 60/934,954, filed Jun. 6, 2007, and 60/934,929, filed Jun. 14, 2007;

(10) International Application No. PCT/US2008/055357, filed Feb. 28, 2008, which claims the benefit of U.S. Provisional Patent Application Nos. 60/934,930, filed Mar. 16, 2007, 60/934,940, filed Mar. 2, 2007, 60/934,954, filed Jun. 6, 2007, 60/934,929, filed Jun. 14, 2007, and 60/934,965, filed Jun. 22, 2007;

(11) International Application No. PCT/US2008/055378, filed Feb. 28, 2008, which claims the benefit of U.S. Provisional Patent Application Nos. 60/934,930, filed Mar. 16, 2007, 60/934,940, filed Mar. 2, 2007, and 60/949,443, filed Jul. 12, 2007;

(12) International Application No. PCT/US2008/055372, filed Feb. 28, 2008, which claims the benefit of U.S. Provisional Patent Application Nos. 60/934,930, filed Mar. 16, 2007, 60/934,940, filed Mar. 2, 2007, and 60/949,444, filed Jul. 12, 2007;

(13) International Application No. PCT/US2008/055345, filed Feb. 28, 2008, which claims the benefit of U.S. Provisional Patent Application Nos. 60/934,930, filed Mar. 16, 2007, 60/934,940, filed Mar. 2, 2007, and 60/949,448, filed Jul. 12, 2007;

(14) International Application No. PCT/US2008/055377, filed Feb. 28, 2008, which claims the benefit of U.S. Provisional Patent Application Nos. 60/934,930, filed Mar. 16, 2007, 60/934,940, filed Mar. 2, 2007, 60/949,450, filed Jul. 12, 2007, 60/951,167, filed Jul. 20, 2007, 60/951,168, filed Jul. 20, 2007, and 60/951,170, filed Jul. 20, 2007;

(15) International Application No. PCT/US2008/055376, filed Feb. 28, 2008, which claims the benefit of U.S. Provisional Patent Application Nos. 60/934,930, filed Mar. 16, 2007, 60/934,940, filed Mar. 2, 2007, 60/952,191, filed Jul. 26, 2007, 60/952,188, filed Jul. 26, 2007, 60/952,192, filed Jul. 26, 2007, and 60/952,499, filed Jul. 27, 2007;

(16) International Application No. PCT/US2008/055373, filed Feb. 28, 2008, which claims the benefit of U.S. Provisional Patent Application Nos. 60/934,930, filed Mar. 16, 2007, 60/934,940, filed Mar. 2, 2007, 60/952,191, filed Jul. 26, 2007, 60/952,188, filed Jul. 26, 2007, 60/952,192, filed Jul. 26, 2007, 60/952,499, filed Jul. 27, 2007, and 60/953,873, filed Aug. 3, 2007;

(17) International Application No. PCT/US2008/055385, filed Feb. 28, 2008, which claims the benefit of U.S. Provisional Patent Application Nos. 60/934,930, filed Mar. 16, 2007, 60/934,940, filed Mar. 2, 2007, and 60/956,093, filed Aug. 15, 2007;

(18) International Application No. PCT/US2008/055386, filed Feb. 28, 2008, which claims the benefit of U.S. Provisional Patent Application Nos. 60/934,930, filed Mar. 16, 2007, 60/934,940, filed Mar. 2, 2007, and 60/970,414, filed Sep. 6, 2007;

(19) International Application No. PCT/US2008/055382, filed Feb. 28, 2008, which claims the benefit of U.S. Provisional Patent Application Nos. 60/934,930, filed Mar. 16, 2007, 60/934,940, filed Mar. 2, 2007, and 60/956,679, filed Aug. 17, 2007;

(20) International Application No. PCT/US2008/055333, filed Feb. 28, 2008, which claims the benefit of U.S. Provisional Patent Application Nos. 60/934,930, filed Mar. 16, 2007, 60/934,940, filed Mar. 2, 2007, and 60/970,796, filed Sep. 7, 2007;

(21) International Application No. PCT/US2008/055341, filed Feb. 28, 2008, which claims the benefit of U.S. Provisional Patent Application Nos. 60/934,930, filed Mar. 16, 2007, 60/934,940, filed Mar. 2, 2007, and 60/970,858, filed Sep. 7, 2007;

(22) International Application No. PCT/US2008/055350, filed Feb. 28, 2008, which claims the benefit of U.S. Provisional Patent Application Nos. 60/934,930, filed Mar. 16, 2007, 60/934,940, filed Mar. 2, 2007, and 60/971,476, filed Sep. 11, 2007;

(23) International Application No. PCT/US2008/055356, filed Feb. 28, 2008, which claims the benefit of U.S. Provisional Patent Application Nos. 60/934,930, filed Mar. 16, 2007, 60/934,940, filed Mar. 2, 2007, and 60/971,874, filed Sep. 12, 2007;

(24) International Application No. PCT/US2008/055366, filed Feb. 28, 2008, which claims the benefit of U.S. Provisional Patent Application Nos. 60/934,930, filed Mar. 16, 2007, 60/934,940, filed Mar. 2, 2007, and 60/973,397, filed Sep. 18, 2007;

(25) International Application No. PCT/US2008/055339, filed Feb. 28, 2008, which claims the benefit of U.S. Provisional Patent Application Nos. 60/934,930, filed Mar. 16, 2007, 60/934,940, filed Mar. 2, 2007, 60/973,398, filed Sep. 18, 2007, 60/013,212, filed Dec. 12, 2007, 60/013,239, filed Dec. 12, 2007, and 60/973,397, filed Sep. 18, 2007;

(26) International Application No. PCT/US2008/055365, filed Feb. 28, 2008, which claims the benefit of U.S. Provisional Patent Application Nos. 60/934,930, filed Mar. 16, 2007, 60/934,940, filed Mar. 2, 2007, and 60/983,179, filed Oct. 27, 2007;

(27) International Application No. PCT/US2008/055340, filed Feb. 28, 2008, which claims the benefit of U.S. Provisional Patent Application Nos. 60/934,930, filed Mar. 16, 2007, 60/934,940, filed Mar. 2, 2007, 60/983,183, filed Oct. 27, 2007, 60/983,182, filed Oct. 27, 2007, 60/983,180, filed Oct. 27, 2007, 60/983,181, filed Oct. 27, 2007, 60/986,910, filed Nov. 9, 2007, 60/986,822, filed Nov. 9, 2007, and 60/986,893, filed Nov. 9, 2007;

(28) International Application No. PCT/US2008/055505, filed Feb. 29, 2008, which claims the benefit of U.S. Provisional Patent Application Nos. 60/934,930, filed Mar. 16, 2007, 60/934,940, filed Mar. 2, 2007, and 60/986,913, filed Nov. 9, 2007;

(29) International Application No. PCT/US2008/055556, filed Feb. 29, 2008, which claims the benefit of U.S. Provisional Patent Application Nos. 60/934,930, filed Mar. 16, 2007, 60/934,940, filed Mar. 2, 2007, and 61/021,491, filed Nov. 16, 2007;

(30) International Application No. PCT/US2008/055515, filed Feb. 29, 2008, which claims the benefit of U.S. Provisional Patent Application Nos. 60/934,930, filed Mar. 16, 2007, 60/934,940, filed Mar. 2, 2007, and 60/989,419, filed Nov. 20, 2007;

(31) International Application No. PCT/US2008/055599, filed Mar. 3, 2008, which claims the benefit of U.S. Provisional Patent Application Nos. 60/934,930, filed Mar. 16, 2007, 60/934,940, filed Mar. 2, 2007, and 60/986,916, filed Nov. 9, 2007;

(32) International Application No. PCT/US2008/055601, filed Mar. 3, 2008, which claims the benefit of U.S. Provisional Patent Application Nos. 60/934,930, filed Mar. 16, 2007, 60/934,940, filed Mar. 2, 2007, and 60/986,919, filed Nov. 9, 2007;

(33) International Application No. PCT/US2008/055603, filed Mar. 3, 2008, which claims the benefit of U.S. Provisional Patent Application Nos. 60/934,930, filed Mar. 16, 2007, 60/934,940, filed Mar. 2, 2007, and 60/987,733, filed Nov. 13, 2007;

(34) International Application No. PCT/US2008/055606, filed Mar. 3, 2008, which claims the benefit of U.S. Provisional Patent Application Nos. 60/934,930, filed Mar. 16, 2007, 60/934,940, filed Mar. 2, 2007, and 60/987,737, filed Nov. 13, 2007;

(35) International Application No. PCT/US2008/055548, filed Feb. 29, 2008, which claims the benefit of U.S. Provisional Patent Application Nos. 60/934,930, filed Mar. 16, 2007, 60/934,940, filed Mar. 2, 2007, 60/987,740, filed Nov. 13, 2007, 60/988,076, filed Nov. 14, 2007, 60/988,079, filed Nov. 14, 2007, 60/988,082, filed Nov. 14, 2007, and 60/988,083, filed Nov. 14, 2007;

(36) International Application No. PCT/US2008/055611, filed Mar. 3, 2008, which claims the benefit of U.S. Provisional Patent Application Nos. 60/934,930, filed Mar. 16, 2007, 60/934,940, filed Mar. 2, 2007, and 61/013,979, filed Dec. 14, 2007;

(37) International Application No. PCT/US2008/055615, filed Mar. 3, 2008, which claims the benefit of U.S. Provisional Patent Application Nos. 60/934,930, filed Mar. 16, 2007, 60/934,940, filed Mar. 2, 2007, and 60/988,395, filed Nov. 15, 2007;

(38) International Application No. PCT/US2008/055709, filed Mar. 3, 2008, which claims the benefit of U.S. Provisional Patent Application Nos. 60/934,930, filed Mar. 16, 2007, 60/934,940, filed Mar. 2, 2007, 60/988,397, filed Nov. 15, 2007, and 60/988,398, filed Nov. 15, 2007;

(39) International Application No. PCT/US2008/055618, filed Mar. 3, 2008, which claims the benefit of U.S. Provisional Patent Application Nos. 60/934,930, filed Mar. 16, 2007, 60/934,940, filed Mar. 2, 2007, and 60/988,399, filed Nov. 15, 2007;

(40) International Application No. PCT/US2008/055644, filed Mar. 3, 2008, which claims the benefit of U.S. Provisional Patent Application Nos. 60/934,930, filed Mar. 16, 2007, 60/934,940, filed Mar. 2, 2007, 60/988,400, filed Nov. 15, 2007, 60/988,401, filed Nov. 15, 2007, and 60/988,402, filed Nov. 15, 2007;

(41) International Application No. PCT/US2008/055651, filed Mar. 3, 2008, which claims the benefit of U.S. Provisional Patent Application Nos. 60/934,930, filed Mar. 16, 2007, 60/934,940, filed Mar. 2, 2007, 60/986,944, filed Nov. 9, 2007, 60/988,403, filed Nov. 15, 2007, and 61/013,981, filed Dec. 14, 2007;

(42) International Application No. PCT/US2008/055649, filed Mar. 3, 2008, which claims the benefit of U.S. Provisional Patent Application Nos. 60/934,930, filed Mar. 16, 2007, 60/934,940, filed Mar. 2, 2007, and 61/015,611, filed Dec. 20, 2007;

(43) International Application No. PCT/US2008/055711, filed Mar. 3, 2008, which claims the benefit of U.S. Provisional Patent Application Nos. 60/934,930, filed Mar. 16, 2007, 60/934,940, filed Mar. 2, 2007, 60/988,405, filed Nov. 15, 2007, 60/989,427, filed Nov. 20, 2007, 60/989,424, filed Nov. 20, 2007, and 60/989,428, filed Nov. 20, 2007;

(44) International Application No. PCT/US2008/055635, filed Mar. 3, 2008, which claims the benefit of U.S. Provisional Patent Application Nos. 60/934,930, filed Mar. 16, 2007, 60/934,940, filed Mar. 2, 2007, and 61/014,305, filed Dec. 17, 2007;

(45) International Application No. PCT/US2008/055524, filed Feb. 29, 2008, which claims the benefit of U.S. Provisional Patent Application Nos. 60/934,930, filed Mar. 16, 2007, 60/934,940, filed Mar. 2, 2007, and 61/014,730, filed Dec. 18, 2007;

(46) International Application No. PCT/US2008/055572, filed Mar. 3, 2008, which claims the benefit of U.S. Provisional Patent Application Nos. 60/934,930, filed Mar. 16, 2007, 60/934,940, filed Mar. 2, 2007, and 61/015,164, filed Dec. 19, 2007;

(47) International Application No. PCT/US2008/055627, filed Mar. 3, 2008, which claims the benefit of U.S. Provisional Patent Application Nos. 60/934,930, filed Mar. 16, 2007, 60/934,940, filed Mar. 2, 2007, and 61/015,166, filed Dec. 19, 2007;

(48) International Application No. PCT/US2008/055697, filed Mar. 3, 2008, which claims the benefit of U.S. Provisional Patent Application Nos. 60/934,930, filed Mar. 16, 2007, 60/934,940, filed Mar. 2, 2007, and 61/012,401, filed Dec. 7, 2007;

(49) International Application No. PCT/US2008/055662, filed Mar. 3, 2008, which claims the benefit of U.S. Provisional Patent Application Nos. 60/934,930, filed Mar. 16, 2007, 60/934,940, filed Mar. 2, 2007, and 61/016,340, filed Dec. 21, 2007;

(50) International Application No. PCT/US2008/055678, filed Mar. 3, 2008, which claims the benefit of U.S. Provisional Patent Application Nos. 60/934,930, filed Mar. 16, 2007, 60/934,940, filed Mar. 2, 2007, and 60/992,808, filed Dec. 6, 2007;

(51) International Application No. PCT/US2008/055368, filed Feb. 2, 2008, which claims the benefit of U.S. Provisional Patent Application Nos. 60/934,930, filed Mar. 16, 2007, 60/934,940, filed Mar. 2, 2007, and 61/013,982, filed Dec. 14, 2007;

(52) International Application No. PCT/US2008/055676, filed Mar. 3, 2008, which claims the benefit of U.S. Provisional Patent Application Nos. 60/934,930, filed Mar. 16, 2007, 60/934,940, filed Mar. 2, 2007, and 61/014,139, filed Dec. 17, 2007;

(53) International Application No. PCT/US2008/055550, filed Feb. 29, 2008, which claims the benefit of U.S. Provisional Patent Application Nos. 60/934,930, filed Mar. 16, 2007, 60/934,940, filed Mar. 2, 2007, 60/934,921, filed May 24, 2007, 60/932,968, filed May 30, 2007, 60/932,969, filed May 30, 2007, and 60/932,967, filed Jun. 1, 2007;

(54) International Application No. PCT/US2008/055560, filed Feb. 29, 2008, which claims the benefit of U.S. Provisional Patent Application Nos. 60/934,930, filed Mar. 16, 2007, 60/934,940, filed Mar. 2, 2007, 60/934,921, filed May 24, 2007; 60/932,968, filed May 30, 2007, 60/932,969, filed May 30, 2007, 60/932,967, filed Jun. 1, 2007, and 61/014,733 filed Dec. 18, 2007;

(55) International Application No. PCT/US2008/055698, filed Mar. 3, 2008, which claims the benefit of U.S. Provisional Patent Application Nos. 60/934,930, filed Mar. 16, 2007, 60/934,940, filed Mar. 2, 2007, and 61/012,402, filed Dec. 7, 2007;

(56) International Application No. PCT/US2008/055695, filed Mar. 3, 2008, which claims the benefit of U.S. Provisional Patent Application Nos. 60/934,930, filed Mar. 16, 2007, 60/934,940, filed Mar. 2, 2007, and 61/016,343, filed Dec. 21, 2007;

(57) International Application No. PCT/US2008/055701, filed Mar. 3, 2008, which claims the benefit of U.S. Provisional Patent Application Nos. 60/934,930, filed Mar. 16, 2007, 60/934,940, filed Mar. 2, 2007, and 61/015,616, filed Dec. 20, 2007;

(58) International Application No. PCT/US2008/055693, filed Mar. 3, 2008, which claims the benefit of U.S. Provisional Patent Application Nos. 60/934,930, filed Mar. 16, 2007, 60/934,940, filed Mar. 2, 2007, and 61/015,618, filed Dec. 20, 2007;

(59) International Application No. PCT/US2008/055704, filed Mar. 3, 2008, which claims the benefit of U.S. Provisional Patent Application Nos. 60/934,930, filed Mar. 16, 2007, 60/934,940, filed Mar. 2, 2007, and 61/014,734, filed Dec. 18, 2007;

(60) International Application No. PCT/US2008/055708, filed Mar. 3, 2008, which claims the benefit of U.S. Provisional Patent Application Nos. 60/934,930, filed Mar. 16, 2007, 60/934,940, filed Mar. 2, 2007, and 61/015,167, filed Dec. 19, 2007;

(61) International Application No. PCT/US2008/055597, filed Mar. 3, 2008, which claims the benefit of U.S. Provisional Patent Application Nos. 60/934,930, filed Mar. 16, 2007, 60/934,940, filed Mar. 2, 2007, and 61/015,170, filed Dec. 19, 2007;

(62) International Application No. PCT/US2008/055604, filed Mar. 3, 2008, which claims the benefit of U.S. Provisional Patent Application Nos. 60/934,930, filed Mar. 16, 2007, 60/934,940, filed Mar. 2, 2007, and 61/014,735, filed Dec. 18, 2007;

(63) International Application No. PCT/US2008/055608, filed Mar. 3, 2008, which claims the benefit of U.S. Provisional Patent Application Nos. 60/934,930, filed Mar. 16, 2007, 60/934,940, filed Mar. 2, 2007, and 61/014,737, filed Dec. 18, 2007;

(64) International Application No. PCT/US2008/055353, filed Mar. 3, 2008, which claims the benefit of U.S. Provisional Patent Application Nos. 60/934,930, filed Mar. 16, 2007, 60/934,940, filed Mar. 2, 2007, and 60/992,975, filed Dec. 6, 2007;

(65) International Application No. PCT/US2008/055631, filed Mar. 3, 2008, which claims the benefit of U.S. Provisional Patent Application Nos. 60/934,930, filed Mar. 16, 2007, 60/934,940, filed Mar. 2, 2007, and 61/015,171, filed Dec. 19, 2007;

(66) International Application No. PCT/US2008/055563, filed Feb. 29, 2008, which claims the benefit of U.S. Provisional Patent Application Nos. 60/934,930, filed Mar. 16, 2007, 60/934,940, filed Mar. 2, 2007, and 61/015,173, filed Dec. 19, 2007;

(67) International Application No. PCT/US2008/055612, filed Mar. 3, 2008, which claims the benefit of U.S. Provisional Patent Application Nos. 60/934,930, filed Mar. 16, 2007, 60/934,940, filed Mar. 2, 2007, and 61/012,404, filed Dec. 7, 2007;

(68) International Application No. PCT/US2008/055622, filed Mar. 3, 2008, which claims the benefit of U.S. Provisional Patent Application Nos. 60/934,930, filed Mar. 16, 2007, 60/934,940, filed Mar. 2, 2007, and 61/014,738, filed Dec. 18, 2007;

(69) International Application No. PCT/US2008/055625, filed Mar. 3, 2008, which claims the benefit of U.S. Provisional Patent Application Nos. 60/934,930, filed Mar. 16, 2007, 60/934,940, filed Mar. 2, 2007, and 61/015,619, filed Dec. 20, 2007;

(70) International Application No. PCT/US2008/055527, filed Feb. 29, 2008, which claims the benefit of U.S. Provisional Patent Application Nos. 60/934,930, filed Mar. 16, 2007, and 60/934,940, filed Mar. 2, 2007;

(71) International Application No. PCT/US2008/055533, filed Feb. 29, 2008, which claims the benefit of U.S. Provisional Patent Application Nos. 60/934,930, filed Mar. 16, 2007, 60/934,940, filed Mar. 2, 2007, and 61/016,319, filed Dec. 21, 2007;

(72) International Application No. PCT/US2008/055554, filed Feb. 29, 2008, which claims the benefit of U.S. Provisional Patent Application Nos. 60/934,930, filed Mar. 16, 2007, and 60/934,940, filed Mar. 2, 2007;

(73) International Application No. PCT/US2008/055511, filed Feb. 29, 2008, which claims the benefit of U.S. Provisional Patent Application Nos. 60/934,930, filed Mar. 16, 2007, and 60/934,940, filed Mar. 2, 2007;

(74) International Application No. PCT/US2008/055532, filed Feb. 29, 2008, which claims the benefit of U.S. Provisional Patent Application Nos. 60/934,930, filed Mar. 16, 2007, and 60/934,940, filed Mar. 2, 2007;

(75) International Application No. PCT/US2008/055516, filed Feb. 29, 2008, which claims the benefit of U.S. Provisional Patent Application Nos. 60/934,930, filed Mar. 16, 2007, and 60/934,940, filed Mar. 2, 2007;

(76) International Application No. PCT/US2008/055551, filed Feb. 29, 2008, which claims the benefit of U.S. Provisional Patent Application Nos. 60/934,930, filed Mar. 16, 2007, and 60/934,940, filed Mar. 2, 2007;

(77) International Application No. PCT/US2008/055519, filed Feb. 29, 2008, which claims the benefit of U.S. Provisional Patent Application Nos. 60/934,930, filed Mar. 16, 2007, and 60/934,940, filed Mar. 2, 2007;

(78) International Application No. PCT/US2008/055542, filed Feb. 29, 2008, which claims the benefit of U.S. Provisional Patent Application Nos. 60/934,930, filed Mar. 16, 2007, and 60/934,940, filed Mar. 2, 2007; and

(79) International Application No. PCT/US2008/055526, filed Feb. 29, 2008, which claims the benefit of U.S. Provisional Patent Application Nos. 60/934,930, filed Mar. 16, 2007, and 60/934,940, filed Mar. 2, 2007.

All of the above identified applications are incorporated by reference in their entirety.

### TECHNICAL FIELD

The present disclosure provides RNA molecules, for example, meroduplex ribonucleic acid molecules (mdRNA) and blunt ended double-stranded ribonucleic acid molecules, which are capable of decreasing or silencing expression of a target gene or family of genes. An mdRNA of this disclosure comprises at least three strands that combine to form at least two non-overlapping double-stranded regions separated by a nick or gap wherein at least one strand is complementary to a particular mRNA(s). Also provided are methods of decreasing expression of a target gene or family of genes in a cell or in a subject to treat a disease or condition associated with the target gene or family of genes.

### BACKGROUND

RNA interference (RNAi) refers to the cellular process of sequence specific, post-transcriptional gene silencing in animals mediated by small inhibitory nucleic acid molecules, such as a double-stranded RNA (dsRNA) that is homologous to a portion of a targeted messenger RNA (Fire et al., Nature 391:806, 1998; Hamilton et al., Science 286:950-951, 1999). RNAi has been observed in a variety of organisms, including mammals (Fire et al., Nature 391:806, 1998; Bahramian and Zarbl, Mol. Cell. Biol. 19:274-283, 1999; Wianny and Goetz, Nature Cell Biol. 2:70, 1999). RNAi can be induced by introducing an exogenous synthetic 21-nucleotide RNA duplex into cultured mammalian cells (Elbashir et al., Nature 411:494, 2001a).

The mechanism by which dsRNA mediates targeted gene-silencing can be described as involving two steps. The first step involves degradation of long dsRNAs by a ribonuclease III-like enzyme, referred to as Dicer, into short interfering RNAs (siRNAs) having from 21 to 23 nucleotides with double-stranded regions of about 19 base pairs and a two nucleotide, generally, overhang at each 3′-end (Berstein et al., Nature 409:363, 2001; Elbashir et al., Genes Dev. 15:188, 2001b; and Kim et al., Nature Biotech. 23:222, 2005). The second step of RNAi gene-silencing involves activation of a multi-component nuclease having one strand (guide or antisense strand) from the siRNA and an Argonaute protein to form an RNA-induced silencing complex (“RISC”) (Elbashir et al., Genes Dev. 15:188, 2001). Argonaute initially associates with a double-stranded siRNA and then endonucleolytically cleaves the non-incorporated strand (passenger or sense strand) to facilitate its release due to resulting thermodynamic instability of the cleaved duplex (Leuschner et al., EMBO 7:314, 2006). The guide strand in the activated RISC binds to a complementary target mRNA, which is then cleaved by the RISC to promote gene silencing. Cleavage of the target RNA occurs in the middle of the target region that is complementary to the guide strand (Elbashir et al., 2001b).

There continues to be a need for alternative effective therapeutic modalities useful for treating or preventing diseases or disorders in which reduced expression of a gene or family of genes (gene silencing) would be beneficial. The present disclosure meets such needs, and further provides other related advantages.

### BRIEF SUMMARY

Briefly, the present disclosure provides nicked or gapped double-stranded RNA (dsRNA) comprising at least three strands, and blunt ended double-stranded RNA having continuous strands (i.e., not nicked or gapped) that are suitable as substrate for Dicer or as RISC activators to modify expression of a target messenger RNA (mRNA).

In one aspect, the instant disclosure provides meroduplex mdRNA molecules, comprising a first strand that is complementary to a human mRNA nucleotide sequence, and a second strand and a third strand that are each complementary to non-overlapping regions of the first strand, wherein the second strand and third strands can anneal with the first strand to form at least two double-stranded regions spaced apart by up to 10 nucleotides and thereby forming a gap between the second and third strands, and wherein (a) the mdRNA molecule optionally includes at least one double-stranded region of 5 base pairs to 13 base pairs, or (b) the double-stranded regions combined total about 15 base pairs to about 40 base pairs and the mdRNA molecule optionally has blunt ends. In certain embodiments, the first strand is about 15 to about 40 nucleotides in length, and the second and third strands are each, individually, about 5 to about 20 nucleotides, wherein the combined length of the second and third strands is about 15 nucleotides to about 40 nucleotides. In other embodiments, the first strand is about 15 to about 40 nucleotides in length and is complementary to at least 15, 16, 17, 18, 19, 20, 21, 22, 23, 24, 25, 26, 27, 28, 29, 30, 31, 32, 33, 34, 35, 36, 37, 38, 39, or 40 contiguous nucleotides of a human mRNA nucleotide sequence. In still further embodiments, the first strand is about 15 to about 40 nucleotides in length and is at least 70%, 75%, 80%, 85%, 90%, 95%, 96%, 97%, 98%, or 99% identical to a sequence that is complementary to at least 15, 16, 17, 18, 19, 20, 21, 22, 23, 24, 25, 26, 27, 28, 29, 30, 31, 32, 33, 34, 35, 36, 37, 38, 39, or 40 contiguous nucleotides of a human mRNA nucleotide sequence.

In other embodiments, the mdRNA is a RISC activator (e.g., the first strand has about 15 nucleotides to about 25 nucleotides) or a Dicer substrate (e.g., the first strand has about 26 nucleotides to about 40 nucleotides). In some embodiments, the gap comprises at least one to ten unpaired nucleotides in the first strand positioned between the double-stranded regions formed by the second and third strands when annealed to the first strand. In other embodiments, the gap is a nick. In certain embodiments, the nick or gap is located 10 nucleotides from the 5′-end of the first (antisense) strand or at the Argonaute cleavage site. In another embodiment, the meroduplex nick or gap is positioned such that the thermal stability is maximized for the first and second strand duplex and for the first and third strand duplex as compared to the thermal stability of such meroduplexes having a nick or gap in a different position.

In another aspect, the instant disclosure provides mdRNA molecules having a first strand that is complementary to a human mRNA nucleotide sequence, and a second strand and a third strand that are each complementary to non-overlapping regions of the first strand, wherein the second strand and third strand can anneal with the first strand to form at least two double-stranded regions spaced apart by up to 10 nucleotides and thereby forming a gap between the second and third strands, and wherein (a) the mdRNA molecule includes at least one double-stranded region of 5 base pairs to 13 base pairs or (b) the double-stranded regions combined total about 15 base pairs to about 40 base pairs and the mdRNA molecule optionally has blunt ends; and wherein at least one pyrimidine of the mdRNA comprises a pyrimidine nucleoside according to Formula I or II:

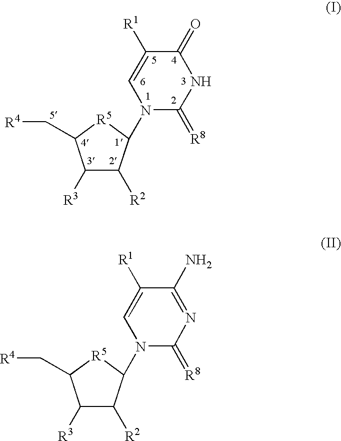

wherein

R¹ and R² are each independently a —H, —OH, —OCH₃, —OCH₂OCH₂CH₃, —OCH₂CH₂OCH₃, halogen, substituted or unsubstituted C₁-C₁₀ alkyl, alkoxy, alkoxyalkyl, hydroxyalkyl, carboxyalkyl, alkylsulfonylamino, aminoalkyl, dialkylamino, alkylaminoalkyl, dialkylaminoalkyl, haloalkyl, trifluoromethyl, cycloalkyl, (cycloalkyl)alkyl, substituted or unsubstituted C₂-C₁₀ alkenyl, substituted or unsubstituted —O-allyl, —O—CH₂CH═CH₂, —O—CH═CHCH₃, substituted or unsubstituted C₂-C₁₀ alkynyl, carbamoyl, carbamyl, carboxy, carbonylamino, substituted or unsubstituted aryl, substituted or unsubstituted aralkyl, —NH₂, —NO₂, —C≡N, or heterocyclo group;

R³ and R⁴ are each independently a hydroxyl, a protected hydroxyl, a phosphate, or an internucleoside linking group; and

R⁵ and R⁸ are independently O or S.

In certain embodiments, at least one nucleoside is according to Formula I in which R¹ is methyl and R² is —OH. In certain related embodiments, at least one uridine of the dsRNA molecule is replaced with a nucleoside according to Formula I in which R¹ is methyl and R² is —OH, or R¹ is methyl, R² is —OH, and R⁸ is S. In some embodiments, at least one R¹ is a C₁-C₅ alkyl, such as methyl. In some embodiments, at least one R² is selected from 2′-O—(C₁-C₅) alkyl, 2′-O-methyl, 2′-OCH₂OCH₂CH₃, 2′-OCH₂CH₂OCH₃, 2′-O-allyl, or fluoro.

In some embodiments, at least one pyrimidine nucleoside of the mdRNA molecule is a locked nucleic acid (LNA) in the form of a bicyclic sugar, wherein R² is oxygen, and the 2′-O and 4′-C form an oxymethylene bridge on the same ribose ring (e.g., a 5-methyluridine LNA) or is a G clamp. In other embodiments, one or more of the nucleosides are according to Formula I in which R¹ is methyl and R² is a 2′-O—(C₁-C₅) alkyl, such as 2′-O-methyl.

In some embodiments, the gap comprises at least one unpaired nucleotide in the first strand positioned between the double-stranded regions formed by the second and third strands when annealed to the first strand, or the gap is a nick. In certain embodiments, the nick or gap is located 10 nucleotides from the 5′-end of the first strand or at the Argonaute cleavage site. In another embodiment, the meroduplex nick or gap is positioned such that the thermal stability is maximized for the first and second strand duplex and for the first and third strand duplex as compared to the thermal stability of such meroduplexes having a nick or gap in a different position.

In still another aspect, the instant disclosure provides for an mdRNA having one more hydroxymethyl modified nucleomonomer(s) (see chemical formulas below—monomers D, F, G, H, I, and J.). Hereunder as one such example is an acyclic nucleomonomer, more preferably an acyclic monomer selected from the group consisting of monomers D, F, G, H, I, and J. Additional monomers that may be incorporated into an mdRNA of this disclosure include:

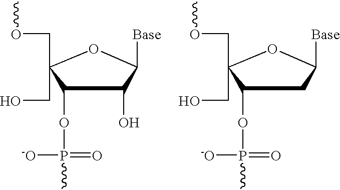

In still another aspect, the instant disclosure provides a method for reducing the expression of a human gene in a cell, comprising administering an mdRNA molecule to a cell expressing the gene, wherein the mdRNA molecule is capable of specifically binding to an mRNA and thereby reducing the gene's level of expression in the cell. In a related aspect, there is provided a method of treating or preventing a disease associated with the expression of a gene or family of genes in a subject by administering an mdRNA molecule of this disclosure. In certain embodiments, the cell or subject is human. In certain embodiments, the disease is cancer, a metabolic disease or an inflammatory disease.

In any of the aspects of this disclosure, some embodiments provide an mdRNA molecule having a 5-methyluridine (ribothymidine), a 2-thioribothymidine, or 2′-O-methyl-5-methyluridine in place of at least one uridine on the first, second, or third strand, or in place of each and every uridine on the first, second, or third strand. In further embodiments, the mdRNA further comprises one or more non-standard nucleoside, such as a deoxyuridine, locked nucleic acid (LNA) molecule, or a universal-binding nucleotide, or a G clamp. Exemplary universal-binding nucleotides include C-phenyl, C-naphthyl, inosine, azole carboxamide, 1-β-D-ribofuranosyl-4-nitroindole, 1-β-D-ribofuranosyl-5-nitroindole, 1-β-D-ribofuranosyl-6-nitroindole, or 1-β-D-ribofuranosyl-3-nitropyrrole.

In some embodiments, the mdRNA molecule further comprises a 2′-sugar substitution, such as a 2′-O-methyl, 2′-O-methoxyethyl, 2′-O-2-methoxyethyl, 2′-O-allyl, or halogen (e.g., 2′-fluoro). In certain embodiments, the mdRNA molecule further comprises a terminal cap substituent on one or both ends of one or more of the first strand, second strand, or third strand, such as independently an alkyl, abasic, deoxy abasic, glyceryl, dinucleotide, acyclic nucleotide, or inverted deoxynucleotide moiety. In other embodiments, the mdRNA molecule further comprises at least one modified internucleoside linkage, such as independently a phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, thionophosphoramidate, thionoalkylphosphonate, thionoalkylphosphotriester, selenophosphate, or boranophosphate linkage.

In any of the aspects of this disclosure, some embodiments provide mdRNA comprising an overhang of one to four nucleotides on at least one 3′-end that is not part of the gap, such as at least one deoxyribonucleotide or two deoxyribonucleotides (e.g., thymidine). In some embodiments, at least one or two 5′-terminal ribonucleotide(s) of the second strand within the double-stranded region comprises a 2′-sugar substitution. In related embodiments, at least one or two 5′-terminal ribonucleotide(s) of the first strand within the double-stranded region comprises a 2′-sugar substitution. In other related embodiments, at least one or two 5′-terminal ribonucleotide of the second strand and at least one or two 5′-terminal ribonucleotide of the first strand within the double-stranded regions comprise independent 2′-sugar substitutions. In other embodiments, the mdRNA molecules comprise at least three 5-methyluridines within at least one double-stranded region. In some embodiments, the mdRNA molecules have a blunt end at one or both ends. In other embodiments, the 5′-terminal of the third strand is a hydroxyl or a phosphate.

In one aspect, the instant disclosure provides double-stranded (dsRNA) molecules, comprising a first strand that is complementary to a human mRNA nucleotide sequence, and a second strand that is complementary to the first strand, wherein the double-stranded region is from about 15 base pairs to about 40 base pairs. In certain aspects, the dsRNA molecule has one or more blunt ends. In certain embodiments, the first strand and the second strand are each independently from about 15 to about 40 nucleotides in length. In other embodiments, the first strand is about 15 to about 40 nucleotides in length and is complementary to at least 15, 16, 17, 18, 19, 20, 21, 22, 23, 24, 25, 26, 27, 28, 29, 30, 31, 32, 33, 34, 35, 36, 37, 38, 39, or 40 contiguous nucleotides of a human mRNA nucleotide sequence. In still further embodiments, the first strand is about 15 to about 40 nucleotides in length and is at least 70%, 75%, 80%, 85%, 90%, 95%, 96%, 97%, 98%, or 99% identical to a sequence that is complementary to at least 15, 16, 17, 18, 19, 20, 21, 22, 23, 24, 25, 26, 27, 28, 29, 30, 31, 32, 33, 34, 35, 36, 37, 38, 39, or 40 contiguous nucleotides of a human mRNA nucleotide sequence.

In other embodiments, the dsRNA is a RISC activator (e.g., the first strand has about 15 nucleotides to about 25 nucleotides) or a Dicer substrate (e.g., the first strand has about 26 nucleotides to about 40 nucleotides).

In another aspect, the instant disclosure provides dsRNA molecules having a first strand that is complementary to a human mRNA nucleotide sequence, and a second strand that is complementary to the first strand, wherein the double-stranded region is from about 15 base pairs to about 40 base pairs. In certain embodiments, the dsRNA has one or more blunt ends and at least one pyrimidine of the dsRNA comprises a pyrimidine nucleoside according to Formula I or II:

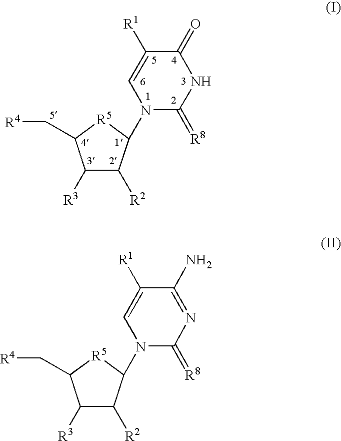

wherein

R¹ and R² are each independently a —H, —OH, —OCH₃, —OCH₂OCH₂CH₃, —OCH₂CH₂OCH₃, halogen, substituted or unsubstituted C₁-C₁₀ alkyl, alkoxy, alkoxyalkyl, hydroxyalkyl, carboxyalkyl, alkylsulfonylamino, aminoalkyl, dialkylamino, alkylaminoalkyl, dialkylaminoalkyl, haloalkyl, trifluoromethyl, cycloalkyl, (cycloalkyl)alkyl, substituted or unsubstituted C₂-C₁₀ alkenyl, substituted or unsubstituted —O-allyl, —O—CH₂CH═CH₂, —O—CH═CHCH₃, substituted or unsubstituted C₂-C₁₀ alkynyl, carbamoyl, carbamyl, carboxy, carbonylamino, substituted or unsubstituted aryl, substituted or unsubstituted aralkyl, —NH₂, —NO₂, —C≡N, or heterocyclo group;

R³ and R⁴ are each independently a hydroxyl, a protected hydroxyl, a phosphate, or an internucleoside linking group; and

R⁵ and R⁸ are independently O or S.

In certain embodiments, at least one nucleoside is according to Formula I in which R¹ is methyl and R² is —OH. In certain related embodiments, at least one uridine of the dsRNA molecule is replaced with a nucleoside according to Formula I in which R¹ is methyl and R² is —OH, or R¹ is methyl, R² is —OH, and R⁸ is S. In some embodiments, the at least one R¹ is a C₁-C₅ alkyl, such as methyl. In some embodiments, at least one R² is selected from 2′-O—(C₁-C₅) alkyl, 2′-O-methyl, 2′-OCH₂OCH₂CH₃, 2′-OCH₂CH₂OCH₃, 2′-O-allyl, or fluoro.

In some embodiments, at least one pyrimidine nucleoside of the dsRNA molecule is a locked nucleic acid (LNA) in the form of a bicyclic sugar, wherein R² is oxygen, and the 2′-O and 4′-C form an oxymethylene bridge on the same ribose ring (e.g., a 5-methyluridine LNA) or is a G clamp. In other embodiments, one or more of the nucleosides are according to Formula I in which R¹ is methyl and R² is a 2′-O—(C₁-C₅) alkyl, such as 2′-O-methyl.

In still another aspect, the instant disclosure provide for a dsRNA having one more hydroxymethyl modified nucleomonomer(s) (see chemical formulas below—monomers D, F, G, H, I, and J.). Hereunder as one such example is an acyclic nucleomonomer, more preferably an acyclic monomer selected from the group consisting of monomers D, F, G, H, I, and J. Additional monomers that may be incorporated into a dsRNA of this disclosure include:

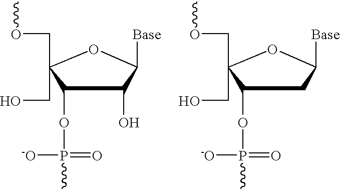

In still another aspect, the instant disclosure provides a method for reducing the expression of a human gene in a cell, comprising administering a dsRNA molecule to a cell expressing the gene, wherein the dsRNA molecule is capable of specifically binding to an mRNA and thereby reducing the gene's level of expression in the cell. In a related aspect, there is provided a method of treating or preventing a disease associated with the expression of a gene or family of genes in a subject by administering a dsRNA molecule of this disclosure. In certain embodiments, the cell or subject is human. In certain embodiments, the disease is cancer, a metabolic disease or inflammatory disease.

In any of the aspects of this disclosure, some embodiments provide dsRNA molecules having a 5-methyluridine (ribothymidine), a 2-thioribothymidine, or 2′-O-methyl-5-methyluridine in place of at least one uridine on the first, second, or third strand, or in place of each and every uridine on the first, second, or third strand.

In further embodiments, the dsRNA further comprises one or more non-standard nucleoside, such as a deoxyuridine, locked nucleic acid (LNA) molecule, or a universal-binding nucleotide, or a G clamp. Exemplary universal-binding nucleotides include C-phenyl, C-naphthyl, inosine, azole carboxamide, 1-β-D-ribofuranosyl-4-nitroindole, 1-β-D-ribofuranosyl-5-nitroindole, 1-β-D-ribofuranosyl-6-nitroindole, or 1-β-D-ribofuranosyl-3-nitropyrrole. In some embodiments, the dsRNA molecules further comprise a 2′-sugar substitution, such as a 2′-O-methyl, 2′-O-methoxyethyl, 2′-O-2-methoxyethyl, 2′-O-allyl, or halogen (e.g., 2′-fluoro).

In certain embodiments, the dsRNA molecules further comprise a terminal cap substituent on one or both ends of one or more of the first strand or second strands, such as independently an alkyl, abasic, deoxy abasic, glyceryl, dinucleotide, acyclic nucleotide, or inverted deoxynucleotide moiety. In other embodiments, the mdRNA molecule further comprises at least one modified internucleoside linkage, such as independently a phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, thionophosphoramidate, thionoalkylphosphonate, thionoalkylphosphotriester, selenophosphate, or boranophosphate linkage.

In any of the aspects of this disclosure, some embodiments provide a dsRNA comprising an overhang of one to four nucleotides on at least one 3′-end, such as at least one deoxyribonucleotide or two deoxyribonucleotides (e.g., thymidine). In some embodiments, at least one or two 5′-terminal ribonucleotide of the second strand within the double-stranded region comprises a 2′-sugar substitution. In related embodiments, at least one or two 5′-terminal ribonucleotide of the first strand within the double-stranded region comprises a 2′-sugar substitution. In other related embodiments, at least one or two 5′-terminal ribonucleotide of the second strand and at least one or two 5′-terminal ribonucleotide of the first strand within the double-stranded region comprise independent 2′-sugar substitutions. In other embodiments, the dsdRNA molecule comprises at least three 5-methyluridines within the double-stranded region. In some embodiments, the dsRNA molecule has a blunt end at one or both ends. In other embodiments, the 5′-terminal of the third strand is a hydroxyl or a phosphate.

### BRIEF DESCRIPTION OF THE DRAWINGS

FIG. 1 shows the average gene silencing activity of intact (first bar), nicked (middle bar), and gapped (last bar) dsRNA Dicer substrate specific for each of 22 different targets (AKT, EGFR, FLT1, FRAP1, HIF1A, 1L17A, IL18, IL6, MAP2K1, MAPK1, MAPK14, PDGFA, PDGFRA, PIKC3A, PKN3, RAF1, SRD5A1, TNF, TNFSF13B, VEGFA, BCR-ABL [b2a2], and BCR-ABL [b3a2]). Each bar is a graphical representation of an average activity of ten different sequences for each target, which is calculated from the data found in Table 1.

FIG. 2 shows knockdown activity for RISC activator lacZ dsRNA (21 nucleotide sense strand/21 nucleotide antisense strand; 21/21), Dicer substrate lacZ dsRNA (25 nucleotide sense strand/27 nucleotide antisense strand; 25/27), and meroduplex lacZ mdRNA (13 nucleotide sense strand and 11 nucleotide sense strand/27 nucleotide antisense strand; 13, 11/27—the sense strand is missing one nucleotide so that a single nucleotide gap is left between the 13 nucleotide and 11 nucleotide sense strands when annealed to the 27 nucleotide antisense strand. Knockdown activities were normalized to a Qneg control dsRNA and presented as a normalized value of Qneg (i.e., Qneg represents 100% or “normal” gene expression levels). A smaller value indicates a greater knockdown effect.

FIG. 3 shows knockdown activity of a RISC activator influenza dsRNA G1498 (21/21) and nicked dsRNA (10, 11/21) at 100 nM. The “wt” designation indicates an unsubstituted RNA molecule; “rT” indicates RNA having each uridine substituted with a ribothymidine; and “p” indicates that the 5′-nucleotide of that strand was phosphorylated. The 21 nucleotide sense and antisense strands of G1498 were individually nicked between the nucleotides 10 and 11 as measured from the 5′-end, and is referred to as 11, 10/21 and 21/10, 11, respectively. The G1498 single stranded 21 nucleotide antisense strand alone (designated AS-only) was used as a control.

FIG. 4 shows knockdown activity of a lacZ dicer substrate (25/27) having a nick in one of each of positions 8 to 14 and a one nucleotide gap at position 13 of the sense strand (counted from the 5′-end). A dideoxy guanosine (ddG) was incorporated at the 5′-end of the 3′-most strand of the nicked or gapped sense sequence at position 13. FIG. 4 discloses SEQ ID NO: 3.

FIG. 5 shows knockdown activity of a dicer substrate influenza dsRNA G1498DS (25/27) and this sequence nicked at one of each of positions 8 to 14 of the sense strand, and shows the activity of these nicked molecules that are also phosphorylated or have a locked nucleic acid substitution.

FIG. 6 shows a dose dependent knockdown activity a dicer substrate influenza dsRNA G1498DS (25/27) and this sequence nicked at position 13 of the sense strand.

FIG. 7 shows knockdown activity of a dicer substrate influenza dsRNA G1498DS having a nick or a gap of one to six nucleotides that begins at any one of positions 8 to 12 of the sense strand.

FIG. 8 shows knockdown activity of a LacZ RISC dsRNA having a nick or a gap of one to six nucleotides that begins at any one of positions 8 to 14 of the sense strand.

FIG. 9 shows knockdown activity of an influenza RISC dsRNA having a nick at any one of positions 8 to 14 of the sense strand and further having one or two locked nucleic acids (LNA) per sense strand. The inserts on the right side of the graph provides a graphic depiction of the meroduplex structures (for clarity, a single antisense strand is shown at the bottom of the grouping with each of the different nicked sense strands above the antisense) having different nick positions with the relative positioning of the LNAs on the sense strands.

FIG. 10 shows knockdown activity of a LacZ dicer substrate dsRNA having a nick at any one of positions 8 to 14 of the sense strand as compared to the same nicked dicer substrates but having a locked nucleic acid substitution.

FIG. 11 shows the percent knockdown in influenza viral titers using influenza specific mdRNA against influenza strain WSN.

FIG. 12 shows the in vivo reduction in PR8 influenza viral titers using influenza specific mdRNA as measured by TCID₅₀.

### DETAILED DESCRIPTION

The instant disclosure provides nicked or gapped double-stranded RNA comprising at least three strands, and blunt ended double-stranded RNA having continuous strands (i.e., not nicked or gapped) that are suitable substrates for Dicer or RISC and, therefore, may be advantageously employed for gene silencing via, for example, the RNA interference pathway. That is, partially duplexed dsRNA molecules described herein (also referred to as “meroduplexes,” which have a nick or gap in at least one strand) and blunt-ended dsRNA are capable of initiating an RNA interference cascade that modifies (e.g., reduces) expression of a target messenger RNA (mRNA), such as a human tumor necrosis factor (TNF) mRNA, vascular endothelial growth factor (VEGF) mRNA, vascular endothelial growth factor receptor (VEGFR) mRNA, epidermal growth factor receptor (EGFR) mRNA, erythroblastic leukemia viral oncogene homolog (ERBB) mRNA, platelet derived growth factor (PDGF) mRNA, platelet derived growth factor receptor (PDGFR) mRNA, breakpoint cluster region (BCR)-abelson murine leukemia viral oncogene homolog (ABL) mRNA, steroid-5-alpha-reductase, alpha polypeptide 1 (SRD5A1) mRNA, steroid-5-alpha-reductase, alpha polypeptide 2 (SRD5A2) mRNA, phosphoinositide-3-kinase, catalytic (PIK3C) mRNA, mitogen-activated protein kinase (MAPK) mRNA, p38 MAPK family mRNA, hypoxia-inducible factor 1 alpha (HIF1A) mRNA, protein kinase N3 (PKN3) mRNA, interleukin 17A (IL17A) mRNA, interleukin 6 (IL6) mRNA, interleukin 18 (IL18) mRNA, tumor necrosis factor (ligand) superfamily member 13b (TNFSF13B) mRNA, mitogen-activated protein kinase 1 (MAPK1) mRNA, v-raf-1 murine leukemia viral oncogene homolog 1 (RAF1) mRNA, v-AKT murine thymoma viral oncogene (AKT) mRNA, FK506 binding protein 12-rapamycin associated protein 1 (FRAP1) mRNA, mitogen-activated protein kinase 2 (MAPK2) mRNA, cyclin-dependent kinase 2 (CDK2) mRNA, ATP-binding cassette, subfamily B, member 1 (ABCB1) mRNA, B-cell CLL/lymphoma 2 (BCL2) mRNA, angiopoietin 2 (ANGPT2) mRNA, checkpoint kinase 1 homolog (CHEK1) mRNA, insulin-like growth factor 1 receptor (IGF1R) mRNA, signal transducer and activator of transcription 3 (STAT3) mRNA, matrix metalloproteinase (MMP) mRNA, folate hydrolase (prostate-specific membrane antigen) 1 (FOLH1) mRNA, v-myc myelocytomatosis viral oncogene homolog (avian) (MYC) mRNA, telomerase RNA component (TERC) mRNA, telomerase reverse transcriptase (TERT) mRNA, protein kinase C, alpha (PRKCA) mRNA, RAS viral (v-ras) oncogene homolog (RAS) mRNA, chemokine (C-X-C motif) ligand or receptor (CXC) mRNA, Wingless-Type MMTV (Murine Mammary Tumor Virus) Integration Site (WNT) mRNA, toll-like receptor (TLR) mRNA, Fc fragment of IgE, low affinity II, receptor for (CD23) (FCER2) mRNA, FOS gene, (FOS, FOSB, FOSL1, OR FOSL2) mRNA, hydroxysteroid (11-beta) dehydrogenase (HSD11B1) mRNA, JUN gene (cJUN, JUNB, or JUND) mRNA, thymidine phosphorylase (TYMP) mRNA, early growth response (EGR) mRNA, zeste homolog 2 (EZH2) mRNA, cyclin D1 (CCND1) mRNA, Fas (TNF receptor superfamily, member 6) (FAS) mRNA, proliferating cell nuclear antigen (PCNA) mRNA, fibroblast growth factor 2 (FGF2) mRNA, tumor growth factor-beta (TGF-β) mRNA, tumor growth factor-beta receptor (TGF-βR) mRNA, tumor-associated calcium signal transducer 1 (TACSTD1) mRNA, Mucin 1 (MUC1) mRNA, protein tyrosine phosphatase, non-receptor-11 (Noonan Syndrome 1) (PTPN11) mRNA, neuregulin 1 (NRG1) mRNA, membrane metallo-endopeptidase (MME) mRNA, CD19 molecule (CD19) mRNA, CD40 molecule, TNF receptor superfamily member 5 (CD40) mRNA, apolipoprotein B (including Ag(x) antigen) (ApoB) mRNA, synuclein, alpha (non A4 component of amyloid precursor) (SNCA) mRNA, silent mating type information regulation 2 homolog (SIRT2) mRNA, histone deacetylase (HDAC) mRNA, membrane-spanning 4-domains, subfamily A, member 1 (MS4A1) mRNA, CD22 molecule (CD22) mRNA, diacylglycerol o-acyltransferase 1 (DGAT1) mRNA, diacylglycerol o-acyltransferase 2 (DGAT2) mRNA, CD3 molecule (CD3) mRNA, proprotein convertase subtilisin-like kexin type 9 (PCSK9) mRNA, MET (Mesenchymal epithelial transition factor) (c-Met proto-oncogene) mRNA, catenin (cadherin-associated protein) (beta-catenin) (CTNNB1) mRNA, inhition of DNA binding proteins (Inhibition of Differentiation Proteins, Dominant Negative Helix-Loop-Helix Protein) (ID, e.g., ID-1) mRNA, protein tyrosine phosphatase, non-receptor type 1 (PTPN1) mRNA, tie-1 (TIE1; tyrosine kinase with immunoglobulin and EGF factor homology domains 1) mRNA, tek tyrosine kinase (TEK) mRNA, fibroblast growth factor receptor (FGFR) mRNA, mitogen-activated protein kinase 3 (MAPK3) mRNA, survivin (BIRC5) mRNA, polo-like kinase family genes (PLK Family; PLK1, PLK2, and PLK3) mRNA, or any one or more combination gene targets identified above.

Meroduplex ribonucleic acid (mdRNA) molecules described herein include a first (antisense) strand that is complementary to a human mRNA nucleotide sequence, along with second and third strands (together forming a gapped sense strand) that are each complementary to non-overlapping regions of the first strand, wherein the second and third strands can anneal with the first strand to form at least two double-stranded regions separated by a gap, and wherein at least one double-stranded region is from about 5 base pairs to about 15 base pairs, or the combined double-stranded regions total about 15 base pairs to about 40 base pairs and the mdRNA is blunt-ended.

The gap can be from 0 nucleotides (i.e., a nick in which only a phosphodiester bond between two nucleotides is broken in a polynucleotide molecule) up to about 10 nucleotides (i.e., the first strand will have at least one unpaired nucleotide). In certain embodiments, the nick or gap is located 10 nucleotides from the 5′-end of the first (antisense) strand or at the Argonaute cleavage site. In another embodiment, the meroduplex nick or gap is positioned such that the thermal stability is maximized for the first and second strand duplex and for the first and third strand duplex as compared to the thermal stability of such meroduplexes having a nick or gap in a different position.

Also provided herein are methods of using such dsRNA to reduce expression of a gene in a cell or to treat or prevent diseases or disorders associated with gene expression, including cancer, metabolic, and inflammatory diseases.

Prior to introducing more detail to this disclosure, it may be helpful to an appreciation thereof to provide definitions of certain terms to be used herein.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean±20% of the indicated range, value, or structure, unless otherwise indicated. As used herein, the terms “include” and “comprise” are open ended and are used synonymously. It should be understood that the terms “a” and “an” as used herein refer to “one or more” of the enumerated components. The use of the alternative (e.g., “or”) should be understood to mean either one, both, or any combination thereof of the alternatives.

As used herein, the term “isolated” means that the referenced material (e.g., nucleic acid molecules of the instant disclosure), is removed from its original environment, such as being separated from some or all of the co-existing materials in a natural environment (e.g., a natural environment may be a cell).

As used herein, “complementary” refers to a nucleic acid molecule that can form hydrogen bond(s) with another nucleic acid molecule or itself by either traditional Watson-Crick base pairing or other non-traditional types of pairing (e.g., Hoogsteen or reversed Hoogsteen hydrogen bonding) between complementary nucleosides or nucleotides. In reference to the nucleic acid molecules of the present disclosure, the binding free energy for a nucleic acid molecule with its complementary sequence is sufficient to allow the relevant function of the nucleic acid molecule to proceed, for example, RNAi activity, and there is a sufficient degree of complementarity to avoid non-specific binding of the nucleic acid molecule (e.g., dsRNA) to non-target sequences under conditions in which specific binding is desired, i.e., under physiological conditions in the case of in vivo assays or therapeutic treatment, or under conditions in which the assays are performed in the case of in vitro assays (e.g., hybridization assays). Determination of binding free energies for nucleic acid molecules is well known in the art (see, e.g., Turner et al., CSH Symp. Quant. Biol. LII:123, 1987; Frier et al., Proc. Nat'l. Acad. Sci. USA 83:9373, 1986; Turner et al., J. Am. Chem. Soc. 109:3783, 1987). Thus, “complementary” or “specifically hybridizable” or “specifically binds” are terms that indicate a sufficient degree of complementarity or precise pairing such that stable and specific binding occurs between a nucleic acid molecule (e.g., RNA) and a DNA or RNA target.

It is understood in the art that a nucleic acid molecule need not be 100% complementary to a target nucleic acid sequence to be specifically hybridizable or to specifically bind. That is, two or more nucleic acid molecules may be less than fully complementary, as indicated by a percentage of contiguous residues in a nucleic acid molecule that can form hydrogen bonds with a second nucleic acid molecule. For example, a first nucleic acid molecule may have 10 nucleotides and a second nucleic acid molecule may have 10 nucleotides, then base pairing of 5, 6, 7, 8, 9, or 10 nucleotides between the first and second nucleic acid molecules, which may or may not form a contiguous double-stranded region, represents 50%, 60%, 70%, 80%, 90%, and 100% complementarity, respectively. In certain embodiments, complementary nucleic acid molecules may have wrongly paired bases—that is, bases that cannot form a traditional Watson-Crick base pair or other non-traditional types of pair (i.e., “mismatched” bases). For instance, complementary nucleic acid molecules may be identified as having a certain number of “mismatches,” such as zero or about 1, about 2, about 3, about 4 or about 5.

“Perfectly” or “fully” complementary nucleic acid molecules means those in which a certain number of nucleotides of a first nucleic acid molecule hydrogen bond (anneal) with the same number of residues in a second nucleic acid molecule to form a contiguous double-stranded region. For example, two or more fully complementary nucleic acid molecule strands can have the same number of nucleotides (i.e., have the same length and form one double-stranded region, with or without an overhang) or have a different number of nucleotides (e.g., one strand may be shorter than but fully contained within another strand or one strand may overhang the other strand).

By “ribonucleic acid” or “RNA” is meant a nucleic acid molecule comprising at least one ribonucleotide molecule. As used herein, “ribonucleotide” refers to a nucleotide with a hydroxyl group at the 2′-position of a β-D-ribofuranose moiety. The term RNA includes double-stranded (ds) RNA, single-stranded (ss) RNA, isolated RNA (such as partially purified RNA, essentially pure RNA, synthetic RNA, recombinantly produced RNA), altered RNA (which differs from naturally occurring RNA by the addition, deletion, substitution or alteration of one or more nucleotides), or any combination thereof. For example, such altered RNA can include addition of non-nucleotide material, such as at one or both ends of an RNA molecule, internally at one or more nucleotides of the RNA, or any combination thereof. Nucleotides in RNA molecules of the instant disclosure can also comprise non-standard nucleotides, such as naturally occurring nucleotides, non-naturally occurring nucleotides, chemically-modified nucleotides, deoxynucleotides, or any combination thereof. These altered RNAs may be referred to as analogs or analogs of RNA containing standard nucleotides (i.e., standard nucleotides, as used herein, are considered to be adenine, cytidine, guanidine, thymidine, and uridine).

The term “dsRNA” as used herein, which includes “mdRNA,” refers to any nucleic acid molecule comprising at least one ribonucleotide molecule and capable of inhibiting or down regulating gene expression, for example, by promoting RNA interference (“RNAi”) or gene silencing in a sequence-specific manner. The dsRNAs and mdRNAs of the instant disclosure may be suitable substrates for Dicer or for association with RISC to mediate gene silencing by RNAi. Examples of dsRNA molecules of this disclosure may be generated with single stranded nucleic acid molecules as presented in the provided Sequence Listing identified herein. One or both strands of the dsRNA can further comprise a terminal phosphate group, such as a 5′-phosphate or 5′, 3′-diphosphate. As used herein, dsRNA molecules, in addition to at least one ribonucleotide, can further include substitutions, chemically-modified nucleotides, and non-nucleotides. In certain embodiments, dsRNA molecules comprise ribonucleotides up to about 100% of the nucleotide positions.

In addition, as used herein, the term dsRNA is meant to be equivalent to other terms used to describe nucleic acid molecules that are capable of mediating sequence specific RNAi, for example, meroduplex RNA (mdRNA), nicked dsRNA (ndsRNA), gapped dsRNA (gdsRNA), short interfering nucleic acid (siNA), siRNA, micro-RNA (miRNA), short hairpin RNA (shRNA), short interfering oligonucleotide, short interfering substituted oligonucleotide, short interfering modified oligonucleotide, chemically-modified dsRNA, post-transcriptional gene silencing RNA (ptgsRNA), or the like. The term “large double-stranded RNA” (“large dsRNA”) refers to any double-stranded RNA longer than about 40 base pairs (bp) to about 100 by or more, particularly up to about 300 by to about 500 bp. The sequence of a large dsRNA may represent a segment of an mRNA or an entire mRNA. A double-stranded structure may be formed by a self-complementary nucleic acid molecule or by annealing of two or more distinct complementary nucleic acid molecule strands.

In one aspect, a dsRNA comprises two separate oligonucleotides, comprising a first strand (antisense) and a second strand (sense), wherein the antisense and sense strands are self-complementary (i.e., each strand comprises a nucleotide sequence that is complementary to a nucleotide sequence in the other strand and the two separate strands form a duplex or double-stranded structure, for example, wherein the double-stranded region is about 15 to about 24 base pairs or about 26 to about 40 base pairs); the antisense strand comprises a nucleotide sequence that is complementary to a nucleotide sequence in a target nucleic acid molecule or a portion thereof; and the sense strand comprises a nucleotide sequence corresponding (i.e., homologous) to the target nucleic acid sequence or a portion thereof (e.g., a sense strand of about 15 to about 25 nucleotides or about 26 to about 40 nucleotides corresponds to the target nucleic acid or a portion thereof).

In another aspect, the dsRNA is assembled from a single oligonucleotide in which the self-complementary sense and antisense strands of the dsRNA are linked together by a nucleic acid based-linker or a non-nucleic acid-based linker. In certain embodiments, the first (antisense) and second (sense) strands of the dsRNA molecule are covalently linked by a nucleotide or non-nucleotide linker as described herein and known in the art. In other embodiments, a first dsRNA molecule is covalently linked to at least one second dsRNA molecule by a nucleotide or non-nucleotide linker known in the art, wherein the first dsRNA molecule can be linked to a plurality of other dsRNA molecules that can be the same or different, or any combination thereof. In another embodiment, the linked dsRNA may include a third strand that forms a meroduplex with the linked dsRNA.

In still another aspect, dsRNA molecules described herein form a meroduplex RNA (mdRNA) having three or more strands such as, for example, an ‘A’ (first or antisense) strand, ‘S1’ (second) strand, and ‘S2’ (third) strand in which the ‘S1’ and ‘S2’ strands are complementary to and form base pairs (bp) with non-overlapping regions of the ‘A’ strand (e.g., an mdRNA can have the form of A:S1S2). The double-stranded region formed by the annealing of the ‘S1’ and ‘A’ strands is distinct from and non-overlapping with the double-stranded region formed by the annealing of the ‘S2’ and ‘A’ strands. An mdRNA molecule is a “gapped” molecule, i.e., it contains a “gap” ranging from 0 nucleotides up to about 10 nucleotides (or a gap of 11, 12, 13, 14, 15, 16, 17, 18, 19, 20, 21, 22, 23, 24, 25, 26, 27, 28, 29, 30, 31, 32, 33, 34 or 35 nucleotides). In one embodiment, the A:S1 duplex is separated from the A:S2 duplex by a gap resulting from at least one unpaired nucleotide (up to about 10 unpaired nucleotides) in the ‘A’ strand that is positioned between the A:S1 duplex and the A:S2 duplex and that is distinct from any one or more unpaired nucleotide at the 3′-end of one or more of the ‘A’, ‘S1’, or ‘S2’ strands. In another embodiment, the A:S1 duplex is separated from the A:S2 duplex by a gap of zero nucleotides (i.e., a nick in which only a phosphodiester bond between two nucleotides is broken or missing in the polynucleotide molecule) between the A:S1 duplex and the A:S2 duplex—which can also be referred to as nicked dsRNA (ndsRNA). For example, A:S1S2 may be comprised of a dsRNA having at least two double-stranded regions that, in combination, total about 14 base pairs to about 40 base pairs wherein the double-stranded regions are separated by a gap of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 nucleotides, optionally having blunt ends, or A:S1S2 may comprise a dsRNA having at least two double-stranded regions spaced apart by up to 10 nucleotides and thereby forming a gap between the second and third strands wherein at least one of the double-stranded regions optionally has from 5 base pairs to 13 base pairs.

A dsRNA or large dsRNA may include a substitution or modification in which the substitution or modification may be in a phosphate backbone bond, a sugar, a base, or a nucleoside. Such nucleoside substitutions can include natural non-standard nucleosides (e.g., 5-methyluridine or 5-methylcytidine or a 2-thioribothymidine), and such backbone, sugar, or nucleoside modifications can include an alkyl or heteroatom substitution or addition, such as a methyl, alkoxyalkyl, halogen, nitrogen or sulfur, or other modifications known in the art.

In addition, as used herein, the term “RNAi” is meant to be equivalent to other terms used to describe sequence specific RNA interference, such as post transcriptional gene silencing, translational inhibition, or epigenetics. For example, dsRNA molecules of this disclosure can be used to epigenetically silence genes at the post-transcriptional level or the pre-transcriptional level or any combination thereof.

As used herein, “target nucleic acid”, “target mRNA”, “target RNA”, and “target gene” refers to any nucleic acid sequence whose expression or activity is to be altered (e.g. a target gene may be one or more of the following: tumor necrosis factor (TNF), vascular endothelial growth factor (VEGF), vascular endothelial growth factor receptor (VEGFR), epidermal growth factor receptor (EGFR), erythroblastic leukemia viral oncogene homolog (ERBB), platelet derived growth factor (PDGF), platelet derived growth factor receptor (PDGFR), breakpoint cluster region (BCR)-abelson murine leukemia viral oncogene homolog (ABL), steroid-5-alpha-reductase, alpha polypeptide 1 (SRD5A1), steroid-5-alpha-reductase, alpha polypeptide 2 (SRD5A2), phosphoinositide-3-kinase, catalytic (PIK3C), mitogen-activated protein kinase (MAPK), p38 MAPK family, hypoxia-inducible factor 1 alpha (HIF1A), protein kinase N3 (PKN3), interleukin 17A (IL17A), interleukin 6 (IL6), interleukin 18 (IL18), tumor necrosis factor (ligand) superfamily member 13b (TNFSF13B), mitogen-activated protein kinase 1 (MAPK1), v-raf-1 murine leukemia viral oncogene homolog 1 (RAF1), v-AKT murine thymoma viral oncogene (AKT), FK506 binding protein 12-rapamycin associated protein 1 (FRAP1), mitogen-activated protein kinase 2 (MAPK2), cyclin-dependent kinase 2 (CDK2), ATP-binding cassette, subfamily B, member 1 (ABCB1), B-cell CLL/lymphoma 2 (BCL2), angiopoietin 2 (ANGPT2), checkpoint kinase 1 homolog (CHEK1), insulin-like growth factor 1 receptor (IGF1R), signal transducer and activator of transcription 3 (STAT3), matrix metalloproteinase (MMP), folate hydrolase (prostate-specific membrane antigen) 1 (FOLH1), v-myc myelocytomatosis viral oncogene homolog (avian) (MYC), telomerase RNA component (TERC), telomerase reverse transcriptase (TERT), protein kinase C, alpha (PRKCA), RAS viral (v-ras) oncogene homolog (RAS), chemokine (C-X-C motif) ligand or receptor (CXC), Wingless-Type MMTV (Murine Mammary Tumor Virus) Integration Site (WNT), toll-like receptor (TLR), Fc fragment of IgE, low affinity II, receptor for (CD23) (FCER2), FOS gene, (FOS, FOSB, FOSL1, OR FOSL2), hydroxysteroid (11-beta) dehydrogenase (HSD11B1), JUN gene (cJUN, JUNB, or JUND), thymidine phosphorylase (TYMP), early growth response (EGR), zeste homolog 2 (EZH2), cyclin D1 (CCND1), Fas (TNF receptor superfamily, member 6) (FAS), proliferating cell nuclear antigen (PCNA), fibroblast growth factor 2 (FGF2), tumor growth factor-beta (TGF-β), tumor growth factor-beta receptor (TGF-βR), tumor-associated calcium signal transducer 1 (TACSTD1), Mucin 1 (MUC1), protein tyrosine phosphatase, non-receptor-11 (Noonan Syndrome 1) (PTPN11), neuregulin 1 (NRG1), membrane metallo-endopeptidase (MME), CD19 molecule (CD19), CD40 molecule, TNF receptor superfamily member 5 (CD40), apolipoprotein B (including Ag(x) antigen) (ApoB), synuclein, alpha (non A4 component of amyloid precursor) (SNCA), silent mating type information regulation 2 homolog (SIRT2), histone deacetylase (HDAC), membrane-spanning 4-domains, subfamily A, member 1 (MS4A1), CD22 molecule (CD22), diacylglycerol o-acyltransferase 1 (DGAT1), diacylglycerol o-acyltransferase 2 (DGAT2), CD3 molecule (CD3), proprotein convertase subtilisin-like kexin type 9 (PCSK9), MET (Mesenchymal epithelial transition factor) (c-Met proto-oncogene), catenin (cadherin-associated protein) (beta-catenin) (CTNNB1), inhition of DNA binding proteins (Inhibition of Differentiation Proteins, Dominant Negative Helix-Loop-Helix Protein) (ID, e.g., ID-1), protein tyrosine phosphatase, non-receptor type 1 (PTPN1), tie-1 (TIE1; tyrosine kinase with immunoglobulin and EGF factor homology domains 1), tek tyrosine kinase (TEK), fibroblast growth factor receptor (FGFR), mitogen-activated protein kinase 3 (MAPK3), survivin (BIRC5), polo-like kinase family genes (PLK Family; PLK1, PLK2, and PLK3). The target nucleic acid can be DNA, RNA, or analogs thereof, and includes single, double, and multi-stranded forms. By “target site” or “target sequence” is meant a sequence within a target nucleic acid (e.g., mRNA) that, when present in an RNA molecule, is “targeted” for cleavage by RNAi and mediated by a dsRNA construct of this disclosure containing a sequence within the antisense strand that is complementary to the target site or sequence.

As used herein, “off-target effect” or “off-target profile” refers to the observed altered expression pattern of one or more genes in a cell or other biological sample not targeted, directly or indirectly, for gene silencing by an mdRNA or dsRNA. For example, an off-target effect can be quantified by using a DNA microarray to determine how many non-target genes have an expression level altered by about two-fold or more in the presence of a candidate mdRNA or dsRNA or analog thereof that is specific for a target sequence. A “minimal off-target effect” means that an mdRNA or dsRNA affects expression by about two-fold or more of about 25% to about 1% of the non-target genes examined or it means that the off-target effect of substituted or modified mdRNA or dsRNA (e.g., having at least one uridine substituted with a 5-methyluridine or 2-thioribothymidine and optionally having at least one nucleotide modified at the 2′-position), is reduced by at least about 1% to about 80% or more as compared to the effect on non-target genes of an unsubstituted or unmodified mdRNA or dsRNA.

By “sense region” or “sense strand” is meant one or more nucleotide sequences of a dsRNA molecule having complementarity to one or more antisense regions of the dsRNA molecule. In addition, the sense region of a dsRNA molecule comprises a nucleic acid sequence having homology with or identity to a target sequence. By “antisense region” or “antisense strand” is meant a nucleotide sequence of a dsRNA molecule having complementarity to a target nucleic acid sequence. In addition, the antisense region of a dsRNA molecule can comprise nucleic acid sequence region having complementarity to one or more sense strands of the dsRNA molecule.

“Analog” as used herein refers to a compound that is structurally similar to a parent compound (e.g., a nucleic acid molecule), but differs slightly in composition (e.g., one atom or functional group is different, added, or removed). The analog may or may not have different chemical or physical properties than the original compound and may or may not have improved biological or chemical activity. For example, the analog may be more hydrophilic or it may have altered activity as compared to a parent compound. The analog may mimic the chemical or biological activity of the parent compound (i.e., it may have similar or identical activity), or, in some cases, may have increased or decreased activity. The analog may be a naturally or non-naturally occurring (e.g., chemically-modified or recombinant) variant of the original compound. An example of an RNA analog is an RNA molecule having a non-standard nucleotide, such as 5-methyuridine or 5-methylcytidine or 2-thioribothymidine, which may impart certain desirable properties (e.g., improve stability, bioavailability, minimize off-target effects or interferon response).

As used herein, the term “universal base” refers to nucleotide base analogs that form base pairs with each of the standard DNA/RNA bases with little discrimination between them. A universal base is thus interchangeable with all of the standard bases when substituted into a nucleotide duplex (see, e.g., Loakes et al., J. Mol. Bio. 270:426, 1997). Exemplary universal bases include C-phenyl, C-naphthyl and other aromatic derivatives, inosine, azole carboxamides, or nitroazole derivatives such as 3-nitropyrrole, 4-nitroindole, 5-nitroindole, and 6-nitroindole (see, e.g., Loakes, Nucleic Acids Res. 29:2437, 2001).

The term “gene” as used herein, especially in the context of “target gene” or “gene target” for RNAi, means a nucleic acid molecule that encodes an RNA or a transcription product of such gene, including a messenger RNA (mRNA, also referred to as structural genes that encode for a polypeptide), an mRNA splice variant of such gene, a functional RNA (fRNA), or non-coding RNA (ncRNA), such as small temporal RNA (stRNA), microRNA (miRNA), small nuclear RNA (snRNA), short interfering RNA (siRNA), small nucleolar RNA (snRNA), ribosomal RNA (rRNA), transfer RNA (tRNA) and precursor RNAs thereof. Such non-coding RNAs can serve as target nucleic acid molecules for dsRNA mediated RNAi to alter the activity of the target RNA involved in functional or regulatory cellular processes.

As used herein, “gene silencing” refers to a partial or complete loss-of-function through targeted inhibition of gene expression in a cell, which may also be referred to as RNAi “knockdown,” “inhibition,” “down-regulation,” or “reduction” of expression of a target gene. Depending on the circumstances and the biological problem to be addressed, it may be preferable to partially reduce gene expression. Alternatively, it might be desirable to reduce gene expression as much as possible. The extent of silencing may be determined by methods described herein and known in the art (see, e.g., PCT Publication No. WO 99/32619; Elbashir et al., EMBO J. 20:6877, 2001). Depending on the assay, quantification of gene expression permits detection of various amounts of inhibition that may be desired in certain embodiments of this disclosure, including prophylactic and therapeutic methods, which will be capable of knocking down target gene expression, in terms of mRNA level or protein level or activity, for example, by equal to or greater than 10%, 30%, 50%, 75% 90%, 95% or 99% of baseline (i.e., normal) or other control levels, including elevated expression levels as may be associated with particular disease states or other conditions targeted for therapy.

As used herein, the term “therapeutically effective amount” means an amount of dsRNA that is sufficient to result in a decrease in severity of disease symptoms, an increase in frequency or duration of disease symptom-free periods, or a prevention of impairment or disability due to the disease, in the subject (e.g., human) to which it is administered. For example, a therapeutically effective amount of dsRNA directed against an mRNA of a target nucleic acid or target gene can reduce or alleviate the signs and/or symptoms of a disease or condition by at least about 20%, at least about 40%, at least about 60%, or at least about 80% relative to untreated subjects. A therapeutically effective amount of a therapeutic compound can decrease, for example, atheromatous plaque size or otherwise ameliorate symptoms in a subject. One of ordinary skill in the art would be able to determine such therapeutically effective amounts based on such factors as the subject's size, the severity of symptoms, and the particular composition or route of administration selected. The nucleic acid molecules of the instant disclosure, individually, or in combination or in conjunction with other drugs, can be used to treat diseases or conditions discussed herein. For example, to treat a particular disease, disorder, or condition, the dsRNA molecules can be administered to a patient or can be administered to other appropriate cells evident to those skilled in the art, individually or in combination with one or more drugs, under conditions suitable for treatment.

In addition, one or more dsRNA may be used to knockdown expression of an mRNA or a related mRNA splice variant. In this regard it is noted that a target gene may be transcribed into two or more mRNA splice variants; and thus, for example, in certain embodiments, knockdown of one mRNA splice variant without affecting the other mRNA splice variant may be desired, or vice versa; or knockdown of all transcription products may be targeted.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure. As described herein, all value ranges are inclusive over the indicated range. Thus, a range of C₁-C₄ will be understood to include the values of 1, 2, 3, and 4, such that C1, C2, C3 and C₄ are included.

The term “alkyl” as used herein refers to saturated straight- or branched-chain aliphatic groups containing from 1-20 carbon atoms, preferably 1-8 carbon atoms and most preferably 1-4 carbon atoms. This definition applies as well to the alkyl portion of alkoxy, alkanoyl and aralkyl groups. The alkyl group may be substituted or unsubstituted. In certain embodiments, the alkyl is a (C₁-C₄) alkyl or methyl.

The term “cycloalkyl” as used herein refers to a saturated cyclic hydrocarbon ring system containing from 3 to 12 carbon atoms that may be optionally substituted. Exemplary embodiments include, but are not limited to, cyclopropyl, cyclobutyl, cyclopentyl and cyclohexyl. In certain embodiments, the cycloalkyl group is cyclopropyl. In another embodiment, the (cycloalkyl)alkyl groups contain from 3 to 12 carbon atoms in the cyclic portion and 1 to 6 carbon atoms in the alkyl portion. In certain embodiments, the (cycloalkyl)alkyl group is cyclopropylmethyl. The alkyl groups are optionally substituted with from one to three substituents selected from the group consisting of halogen, hydroxy and amino.

The terms “alkanoyl” and “alkanoyloxy” as used herein refer, respectively, to —C(O)-alkyl groups and —O—C(═O)— alkyl groups, each optionally containing 2 to 10 carbon atoms. Specific embodiments of alkanoyl and alkanoyloxy groups are acetyl and acetoxy, respectively.

The term “alkenyl” refers to an unsaturated branched, straight-chain or cyclic alkyl group having 2 to 15 carbon atoms and having at least one carbon-carbon double bond derived by the removal of one hydrogen atom from a single carbon atom of a parent alkene. The group may be in either the cis or trans conformation about the double bond(s). Certain embodiments include ethenyl, 1-propenyl, 2-propenyl, 1-methylethenyl, 1-butenyl, 2-butenyl, 3-butenyl, 2-methyl-2-propenyl, 1-pentenyl, 2-pentenyl, 4-pentenyl, 3-methyl-2-butenyl, 1-hexenyl, 2-hexenyl, 1-heptenyl, 2-heptenyl, 1-octenyl, 2-octenyl, 1,3-octadienyl, 2-nonenyl, 1,3-nonadienyl, 2-decenyl, etc., or the like. The alkenyl group may be substituted or unsubstituted.

The term “alkynyl” as used herein refers to an unsaturated branched, straight-chain, or cyclic alkyl group having 2 to 10 carbon atoms and having at least one carbon-carbon triple bond derived by the removal of one hydrogen atom from a single carbon atom of a parent alkyne. Exemplary alkynyls include ethynyl, 1-propynyl, 2-propynyl, 1-butynyl, 2-butynyl, 3-butynyl, 1-pentynyl, 2-pentynyl, 4-pentynyl, 1-octynyl, 6-methyl-1-heptynyl, 2-decynyl, or the like. The alkynyl group may be substituted or unsubstituted.

The term “hydroxyalkyl” alone or in combination, refers to an alkyl group as previously defined, wherein one or several hydrogen atoms, preferably one hydrogen atom has been replaced by a hydroxyl group. Examples include hydroxymethyl, hydroxyethyl and 2-hydroxyethyl.

The term “aminoalkyl” as used herein refers to the group —NRR′, where R and R′ may independently be hydrogen or (C₁-C₄) alkyl.

The term “alkylaminoalkyl” refers to an alkylamino group linked via an alkyl group (i.e., a group having the general structure -alkyl-NH-alkyl or -alkyl-N(alkyl)(alkyl)). Such groups include, but are not limited to, mono- and di-(C₁-C₈ alkyl)aminoC₁-C₈ alkyl, in which each alkyl may be the same or different.

The term “dialkylaminoalkyl” refers to alkylamino groups attached to an alkyl group. Examples include, but are not limited to, N,N-dimethylaminomethyl, N,N-dimethylaminoethyl N,N-dimethylaminopropyl, and the like. The term dialkylaminoalkyl also includes groups where the bridging alkyl moiety is optionally substituted.

The term “haloalkyl” refers to an alkyl group substituted with one or more halo groups, for example chloromethyl, 2-bromoethyl, 3-iodopropyl, trifluoromethyl, perfluoropropyl, 8-chlorononyl, or the like.

The term “carboxyalkyl” as used herein refers to the substituent —R¹⁰—COOH, wherein R¹⁰ is alkylene; and “carbalkoxyalkyl” refers to —R¹⁰—C(═O)OR¹¹, wherein R¹⁰ and R¹¹ are alkylene and alkyl respectively. In certain embodiments, alkyl refers to a saturated straight- or branched-chain hydrocarbyl radical of 1 to 6 carbon atoms such as methyl, ethyl, n-propyl, isopropyl, n-butyl, t-butyl, n-pentyl, 2-methylpentyl, n-hexyl, and so forth. Alkylene is the same as alkyl except that the group is divalent.

The term “alkoxy” includes substituted and unsubstituted alkyl, alkenyl, and alkynyl groups covalently linked to an oxygen atom. In one embodiment, the alkoxy group contains 1 to about 10 carbon atoms. Embodiments of alkoxy groups include, but are not limited to, methoxy, ethoxy, isopropyloxy, propoxy, butoxy, and pentoxy groups. Embodiments of substituted alkoxy groups include halogenated alkoxy groups. In a further embodiment, the alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Exemplary halogen substituted alkoxy groups include, but are not limited to, fluoromethoxy, difluoromethoxy, trifluoromethoxy, chloromethoxy, dichloromethoxy, and trichloromethoxy.

The term “alkoxyalkyl” refers to an alkylene group substituted with an alkoxy group. For example, methoxyethyl (CH₃OCH₂CH₂—) and ethoxymethyl (CH₃CH₂OCH₂—) are both C3 alkoxyalkyl groups.

The term “aryl” as used herein refers to monocyclic or bicyclic aromatic hydrocarbon groups having from 6 to 12 carbon atoms in the ring portion, for example, phenyl, naphthyl, biphenyl and diphenyl groups, each of which may be substituted with, for example, one to four substituents such as alkyl; substituted alkyl as defined above, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy. Specific embodiments of aryl groups in accordance with the present disclosure include phenyl, substituted phenyl, naphthyl, biphenyl, and diphenyl.

The term “aroyl,” as used alone or in combination herein, refers to an aryl radical derived from an aromatic carboxylic acid, such as optionally substituted benzoic or naphthoic acids.

The term “aralkyl” as used herein refers to an aryl group bonded to the 2-pyridinyl ring or the 4-pyridinyl ring through an alkyl group, preferably one containing 1 to 10 carbon atoms. A preferred aralkyl group is benzyl.

The term “carboxy,” as used herein, represents a group of the formula —C(═O)OH or —C(═O)O⁻.

The term “carbonyl” as used herein refers to a group in which an oxygen atom is double-bonded to a carbon atom —C═O.

The term “trifluoromethyl” as used herein refers to —CF₃.

The term “trifluoromethoxy” as used herein refers to —OCF₃.

The term “hydroxyl” as used herein refers to —OH or —O⁻.

The term “nitrile” or “cyano” as used herein refers to the group —CN.

The term “nitro,” as used herein alone or in combination refers to a —NO₂ group.

The term “amino” as used herein refers to the group —NR⁹R⁹, wherein R⁹ may independently be hydrogen, alkyl, aryl, alkoxy, or heteroaryl. The term “aminoalkyl” as used herein represents a more detailed selection as compared to “amino” and refers to the group —NR′R′, wherein R′ may independently be hydrogen or (C₁-C₄) alkyl. The term “dialkylamino” refers to an amino group having two attached alkyl groups that can be the same or different.

The term “alkanoylamino” refers to alkyl, alkenyl or alkynyl groups containing the group —C(═O)— followed by —N(H)—, for example acetylamino, propanoylamino and butanoylamino and the like.

The term “carbonylamino” refers to the group —NR′—CO—CH₂—R′, wherein R′ may be independently selected from hydrogen or (C₁-C₄) alkyl.

The term “carbamoyl” as used herein refers to —O—C(O)NH₂.

The term “carbamyl” as used herein refers to a functional group in which a nitrogen atom is directly bonded to a carbonyl, i.e., as in —NR″C(═O)R″ or —C(═O)NR″R″, wherein R″ can be independently hydrogen, alkyl, substituted alkyl, alkenyl, substituted alkenyl, alkoxy, cycloalkyl, aryl, heterocyclo, or heteroaryl.

The term “alkylsulfonylamino” refers to the group —NHS(O)₂R¹², wherein R¹² is alkyl.

The term “halogen” as used herein refers to bromine, chlorine, fluorine or iodine. In one embodiment, the halogen is fluorine. In another embodiment, the halogen is chlorine.

The term “heterocyclo” refers to an optionally substituted, unsaturated, partially saturated, or fully saturated, aromatic or nonaromatic cyclic group that is a 4 to 7 membered monocyclic, or 7 to 11 membered bicyclic ring system that has at least one heteroatom in at least one carbon atom-containing ring. The substituents on the heterocyclo rings may be selected from those given above for the aryl groups. Each ring of the heterocyclo group containing a heteroatom may have 1, 2, or 3 heteroatoms selected from nitrogen, oxygen or sulfur. Plural heteroatoms in a given heterocyclo ring may be the same or different.

Exemplary monocyclic heterocyclo groups include pyrrolidinyl, pyrrolyl, indolyl, pyrazolyl, imidazolyl, oxazolyl, isoxazolyl, thiazolyl, furyl, tetrahydrofuryl, thienyl, piperidinyl, piperazinyl, azepinyl, pyrimidinyl, pyridazinyl, tetrahydropyranyl, morpholinyl, dioxanyl, triazinyl and triazolyl. Preferred bicyclic heterocyclo groups include benzothiazolyl, benzoxazolyl, benzothienyl, quinolinyl, tetrahydroisoquinolinyl, benzimidazolyl, benzofuryl, indazolyl, benzisothiazolyl, isoindolinyl and tetrahydroquinolinyl. In more detailed embodiments heterocyclo groups may include indolyl, imidazolyl, furyl, thienyl, thiazolyl, pyrrolidyl, pyridyl and pyrimidyl.

“Substituted” refers to a group in which one or more hydrogen atoms are each independently replaced with the same or different substituent(s). Representative substituents include —X, —R⁶, —O—, ═O, —OR, —SR⁶, —S—, ═S, —NR⁶R⁶, ═NR⁶, —CX₃, —CF₃, —CN, —OCN, —SCN, —NO, —NO₂, ═N₂, —N₃, —S(═O)₂O—, —S(═O)₂OH, —S(═O)₂R⁶, —OS(═O)₂O—, —OS(═O)₂OH, —OS(═O)₂R⁶, —P(═O)(O⁻)₂, —P(═O)(OH)(O⁻), —OP(═O)₂(O⁻), —C(—O)R⁶, —C(═S)R⁶, —C(═O)OR⁶, —C(═O)O⁻, —C(═S)OR⁶, —NR⁶—C(═O)—N(R⁶)₂, —NR⁶—C(═S)—N(R⁶)₂, and —C(═NR⁶)NR⁶R⁶, wherein each X is independently a halogen; and each R⁶ is independently hydrogen, halogen, alkyl, aryl, arylalkyl, arylaryl, arylheteroalkyl, heteroaryl, heteroarylalkyl, NR⁷R⁷, —C(═O)R⁷, and —S(═O)₂R⁷; and each R⁷ is independently hydrogen, alkyl, alkanyl, alkynyl, aryl, arylalkyl, arylheteralkyl, arylaryl, heteroaryl or heteroarylalkyl. Aryl containing substituents, whether or not having one or more substitutions, may be attached in a para (p-), meta (m-) or ortho (o-) conformation, or any combination thereof.

Target Genes and Exemplary dsRNA Molecules

More detail regarding TNF and its nucleotide sequence (target mRNA), function(s), diseases and disorders related thereto, and example siRNA sequences are found in the priority document patent application PCT/US2008/055371 and associated sequence listing, the contents of which are hereby incorporated by reference in their entirety.

More detail regarding VEGF and its nucleotide sequence (target mRNA), function(s), diseases and disorders related thereto, and example siRNA sequences are found in the priority document patent applications PCT/US2008/055362 and associated sequence listing; and PCT/US2008/055380 and associated sequence listing, the contents of which are hereby incorporated by reference in their entirety.

More detail regarding VEGFR and its nucleotide sequence (target mRNA), function(s), diseases and disorders related thereto, and example siRNA sequences are found in the priority document patent application PCT/US2008/055370 and associated sequence listing; and PCT/US2008/055383 and associated sequence listing, the contents of which are hereby incorporated by reference in their entirety.

More detail regarding ERBB and EGFR and their nucleotide sequences (target mRNAs), function(s), diseases and disorders related thereto, and example siRNA sequences are found in the priority document patent application PCT/US2008/055375 and associated sequence listing; and PCT/US2008/055360 and associated sequence listing, the contents of which are hereby incorporated by reference in their entirety.

More detail regarding PDGF and its nucleotide sequence (target mRNA), function(s), diseases and disorders related thereto, and example siRNA sequences are found in the priority document patent application PCT/US2008/055374 and associated sequence listing, the contents of which are hereby incorporated by reference in their entirety.

More detail regarding PDGFR and its nucleotide sequence (target mRNA), function(s), diseases and disorders related thereto, and example siRNA sequences are found in the priority document patent application PCT/US2008/055381 and associated sequence listing; and PCT/US2008/055357 and associated sequence listing, the contents of which are hereby incorporated by reference in their entirety.

More detail regarding BCR-ABL and its nucleotide sequence (target mRNA), function(s), diseases and disorders related thereto, and example siRNA sequences are found in the priority document patent application PCT/US2008/055378 and associated sequence listing, the contents of which are hereby incorporated by reference in their entirety.

More detail regarding SRD5A1 and its nucleotide sequence (target mRNA), function(s), diseases and disorders related thereto, and example siRNA sequences are found in the priority document patent application PCT/US2008/055372 and associated sequence listing, the contents of which are hereby incorporated by reference in their entirety.

More detail regarding SRD5A2 and its nucleotide sequence (target mRNA), function(s), diseases and disorders related thereto, and example siRNA sequences are found in the priority document patent application PCT/US2008/055345 and associated sequence listing, the contents of which are hereby incorporated by reference in their entirety.

More detail regarding PIK3C and its nucleotide sequence (target mRNA), function(s), diseases and disorders related thereto, and example siRNA sequences are found in the priority document patent application PCT/US2008/055377 and associated sequence listing, the contents of which are hereby incorporated by reference in their entirety.

More detail regarding MAPK and its nucleotide sequence (target mRNA), function(s), diseases and disorders related thereto, and example siRNA sequences are found in the priority document patent application PCT/US2008/055376 and associated sequence listing; and PCT/US2008/055373 and associated sequence listing, the contents of which are hereby incorporated by reference in their entirety.

More detail regarding HIF1A and its nucleotide sequence (target mRNA), function(s), diseases and disorders related thereto, and example siRNA sequences are found in the priority document patent application PCT/US2008/055385 and associated sequence listing, the contents of which are hereby incorporated by reference in their entirety.

More detail regarding PKN3 and its nucleotide sequence (target mRNA), function(s), diseases and disorders related thereto, and example siRNA sequences are found in the priority document patent application PCT/US2008/055386 and associated sequence listing, the contents of which are hereby incorporated by reference in their entirety.

More detail regarding IL17A and its nucleotide sequence (target mRNA), function(s), diseases and disorders related thereto, and example siRNA sequences are found in the priority document patent application PCT/US2008/055382 and associated sequence listing, the contents of which are hereby incorporated by reference in their entirety.

More detail regarding IL6 and its nucleotide sequence (target mRNA), function(s), diseases and disorders related thereto, and example siRNA sequences are found in the priority document patent application PCT/US2008/055333 and associated sequence listing, the contents of which are hereby incorporated by reference in their entirety.

More detail regarding IL18 and its nucleotide sequence (target mRNA), function(s), diseases and disorders related thereto, and example siRNA sequences are found in the priority document patent application PCT/US2008/055341 and associated sequence listing, the contents of which are hereby incorporated by reference in their entirety.

More detail regarding TNFSF13B and its nucleotide sequence (target mRNA), function(s), diseases and disorders related thereto, and example siRNA sequences are found in the priority document patent application PCT/US2008/055350 and associated sequence listing, the contents of which are hereby incorporated by reference in their entirety.

More detail regarding MAPK1 and its nucleotide sequence (target mRNA), function(s), diseases and disorders related thereto, and example siRNA sequences are found in the priority document patent application PCT/US2008/055356 and associated sequence listing, the contents of which are hereby incorporated by reference in their entirety.

More detail regarding RAF1 and its nucleotide sequence (target mRNA), function(s), diseases and disorders related thereto, and example siRNA sequences are found in the priority document patent application PCT/US2008/055366 and associated sequence listing, the contents of which are hereby incorporated by reference in their entirety.

More detail regarding AKT and its nucleotide sequence (target mRNA), function(s), diseases and disorders related thereto, and example siRNA sequences are found in the priority document patent application PCT/US2008/055339 and associated sequence listing, the contents of which are hereby incorporated by reference in their entirety.

More detail regarding FRAP1 and its nucleotide sequence (target mRNA), function(s), diseases and disorders related thereto, and example siRNA sequences are found in the priority document patent application PCT/US2008/055365 and associated sequence listing, the contents of which are hereby incorporated by reference in their entirety.

More detail regarding MAPK2 and its nucleotide sequence (target mRNA), function(s), diseases and disorders related thereto, and example siRNA sequences are found in the priority document patent application PCT/US2008/055340 and associated sequence listing, the contents of which are hereby incorporated by reference in their entirety.

More detail regarding CDK2 and its nucleotide sequence (target mRNA), function(s), diseases and disorders related thereto, and example siRNA sequences are found in the priority document patent application PCT/US2008/055505 and associated sequence listing, the contents of which are hereby incorporated by reference in their entirety.

More detail regarding ABCB1 and its nucleotide sequence (target mRNA), function(s), diseases and disorders related thereto, and example siRNA sequences are found in the priority document patent application PCT/US2008/055556 and associated sequence listing, the contents of which are hereby incorporated by reference in their entirety.

More detail regarding BCL2 and its nucleotide sequence (target mRNA), function(s), diseases and disorders related thereto, and example siRNA sequences are found in the priority document patent application PCT/US2008/055515 and associated sequence listing, the contents of which are hereby incorporated by reference in their entirety.

More detail regarding ANGPT2 and its nucleotide sequence (target mRNA), function(s), diseases and disorders related thereto, and example siRNA sequences are found in the priority document patent application PCT/US2008/055599 and associated sequence listing, the contents of which are hereby incorporated by reference in their entirety.

More detail regarding CHEK1 and its nucleotide sequence (target mRNA), function(s), diseases and disorders related thereto, and example siRNA sequences are found in the priority document patent application PCT/US2008/055601 and associated sequence listing, the contents of which are hereby incorporated by reference in their entirety.

More detail regarding IGF1R and its nucleotide sequence (target mRNA), function(s), diseases and disorders related thereto, and example siRNA sequences are found in the priority document patent application PCT/US2008/055603 and associated sequence listing, the contents of which are hereby incorporated by reference in their entirety.

More detail regarding STAT3 and its nucleotide sequence (target mRNA), function(s), diseases and disorders related thereto, and example siRNA sequences are found in the priority document patent application PCT/US2008/055606 and associated sequence listing, the contents of which are hereby incorporated by reference in their entirety.

More detail regarding MMP and its nucleotide sequence (target mRNA), function(s), diseases and disorders related thereto, and example siRNA sequences are found in the priority document patent application PCT/U2008/055548 and associated sequence listing, the contents of which are hereby incorporated by reference in their entirety.

More detail regarding FOLH1 and its nucleotide sequence (target mRNA), function(s), diseases and disorders related thereto, and example siRNA sequences are found in the priority document patent application PCT/US2008/055611 and associated sequence listing, the contents of which are hereby incorporated by reference in their entirety.

More detail regarding MYC and its nucleotide sequence (target mRNA), function(s), diseases and disorders related thereto, and example siRNA sequences are found in the priority document patent application PCT/US2008/055615 and associated sequence listing, the contents of which are hereby incorporated by reference in their entirety.

More detail regarding TERC and its nucleotide sequence (target mRNA), function(s), diseases and disorders related thereto, and example siRNA sequences are found in the priority document patent application PCT/US2008/055709 and associated sequence listing, the contents of which are hereby incorporated by reference in their entirety.

More detail regarding TERT and its nucleotide sequence (target mRNA), function(s), diseases and disorders related thereto, and example siRNA sequences are found in the priority document patent application PCT/US2008/055709 and associated sequence listing, the contents of which are hereby incorporated by reference in their entirety.

More detail regarding PRKCA and its nucleotide sequence (target mRNA), function(s), diseases and disorders related thereto, and example siRNA sequences are found in the priority document patent application PCT/US2008/055618 and associated sequence listing, the contents of which are hereby incorporated by reference in their entirety.

More detail regarding RAS and its nucleotide sequence (target mRNA), function(s), diseases and disorders related thereto, and example siRNA sequences are found in the priority document patent application PCT/US2008/055644 and associated sequence listing, the contents of which are hereby incorporated by reference in their entirety.

More detail regarding CXC and its nucleotide sequence (target mRNA), function(s), diseases and disorders related thereto, and example siRNA sequences are found in the priority document patent application PCT/US2008/055651 and associated sequence listing, the contents of which are hereby incorporated by reference in their entirety.

More detail regarding WNT and its nucleotide sequence (target mRNA), function(s), diseases and disorders related thereto, and example siRNA sequences are found in the priority document patent application PCT/US2008/055649 and associated sequence listing, the contents of which are hereby incorporated by reference in their entirety.

More detail regarding TLR and its nucleotide sequence (target mRNA), function(s), diseases and disorders related thereto, and example siRNA sequences are found in the priority document patent application PCT/US2008/055711 and associated sequence listing, the contents of which are hereby incorporated by reference in their entirety.

More detail regarding FCER2 and its nucleotide sequence (target mRNA), function(s), diseases and disorders related thereto, and example siRNA sequences are found in the priority document patent application PCT/US2008/055635 and associated sequence listing, the contents of which are hereby incorporated by reference in their entirety.

More detail regarding FOS (FOS, FOSB, FOSL1, or FOSL2) and its nucleotide sequence (target mRNA), function(s), diseases and disorders related thereto, and example siRNA sequences are found in the priority document patent application PCT/US2008/055524 and associated sequence listing, the contents of which are hereby incorporated by reference in their entirety.

More detail regarding HSD11B1 and its nucleotide sequence (target mRNA), function(s), diseases and disorders related thereto, and example siRNA sequences are found in the priority document patent application PCT/US2008/055672 and associated sequence listing, the contents of which are hereby incorporated by reference in their entirety.

More detail regarding cJUN, JUNB, or JUND and their nucleotide sequences (target mRNAs), functions, diseases and disorders related thereto, and example siRNA sequences are found in the priority document patent application PCT/US2008/055627 and associated sequence listing, the contents of which are hereby incorporated by reference in their entirety.

More detail regarding TYMP and its nucleotide sequence (target mRNA), function(s), diseases and disorders related thereto, and example siRNA sequences are found in the priority document patent application PCT/US2008/055697 and associated sequence listing, the contents of which are hereby incorporated by reference in their entirety.

More detail regarding EGR and its nucleotide sequence (target mRNA), function(s), diseases and disorders related thereto, and example siRNA sequences are found in the priority document patent application PCT/US2008/055662 and associated sequence listing, the contents of which are hereby incorporated by reference in their entirety.

More detail regarding EZH2 and its nucleotide sequence (target mRNA), function(s), diseases and disorders related thereto, and example siRNA sequences are found in the priority document patent application PCT/US2008/055678 and associated sequence listing, the contents of which are hereby incorporated by reference in their entirety.

More detail regarding CCND1 and its nucleotide sequence (target mRNA), function(s), diseases and disorders related thereto, and example siRNA sequences are found in the priority document patent application PCT/US2008/055368 and associated sequence listing, the contents of which are hereby incorporated by reference in their entirety.

More detail regarding FAS and its nucleotide sequence (target mRNA), function(s), diseases and disorders related thereto, and example siRNA sequences are found in the priority document patent application PCT/US2008/055676 and associated sequence listing, the contents of which are hereby incorporated by reference in their entirety.

More detail regarding PCNA and its nucleotide sequence (target mRNA), function(s), diseases and disorders related thereto, and example siRNA sequences are found in the priority document patent application PCT/US2008/055550 and associated sequence listing, the contents of which are hereby incorporated by reference in their entirety.

More detail regarding FGF2 and its nucleotide sequence (target mRNA), function(s), diseases and disorders related thereto, and example siRNA sequences are found in the priority document patent application PCT/US2008/055560 and associated sequence listing, the contents of which are hereby incorporated by reference in their entirety.

More detail regarding TGF-β and its nucleotide sequence (target mRNA), function(s), diseases and disorders related thereto, and example siRNA sequences are found in the priority document patent application PCT/US2008/055698 and associated sequence listing, the contents of which are hereby incorporated by reference in their entirety.

More detail regarding TGF-βR and its nucleotide sequence (target mRNA), function(s), diseases and disorders related thereto, and example siRNA sequences are found in the priority document patent application PCT/US2008/055695 and associated sequence listing, the contents of which are hereby incorporated by reference in their entirety.

More detail regarding TACSTD1 and its nucleotide sequence (target mRNA), function(s), diseases and disorders related thereto, and example siRNA sequences are found in the priority document patent application PCT/US2008/055701 and associated sequence listing, the contents of which are hereby incorporated by reference in their entirety.

More detail regarding MUC1 and its nucleotide sequence (target mRNA), function(s), diseases and disorders related thereto, and example siRNA sequences are found in the priority document patent application PCT/US2008/055693 and associated sequence listing, the contents of which are hereby incorporated by reference in their entirety.

More detail regarding PTPN11 and its nucleotide sequence (target mRNA), function(s), diseases and disorders related thereto, and example siRNA sequences are found in the priority document patent application PCT/US2008/055704 and associated sequence listing, the contents of which are hereby incorporated by reference in their entirety.

More detail regarding NGR1 and its nucleotide sequence (target mRNA), function(s), diseases and disorders related thereto, and example siRNA sequences are found in the priority document patent application PCT/US2008/055708 and associated sequence listing, the contents of which are hereby incorporated by reference in their entirety.

More detail regarding MME and its nucleotide sequence (target mRNA), function(s), diseases and disorders related thereto, and example siRNA sequences are found in the priority document patent application PCT/US2008/055597 and associated sequence listing, the contents of which are hereby incorporated by reference in their entirety.

More detail regarding CD19 and its nucleotide sequence (target mRNA), function(s), diseases and disorders related thereto, and example siRNA sequences are found in the priority document patent application PCT/US2008/055604 and associated sequence listing, the contents of which are hereby incorporated by reference in their entirety.

More detail regarding CD40 and its nucleotide sequence (target mRNA), function(s), diseases and disorders related thereto, and example siRNA sequences are found in the priority document patent application PCT/US2008/055608 and associated sequence listing, the contents of which are hereby incorporated by reference in their entirety.

More detail regarding ApoB and its nucleotide sequence (target mRNA), function(s), diseases and disorders related thereto, and example siRNA sequences are found in the priority document patent application PCT/US2008/055353 and associated sequence listing, the contents of which are hereby incorporated by reference in their entirety.

More detail regarding SNCA and its nucleotide sequence (target mRNA), function(s), diseases and disorders related thereto, and example siRNA sequences are found in the priority document patent application PCT/US2008/055631 and associated sequence listing, the contents of which are hereby incorporated by reference in their entirety.

More detail regarding SIRT2 and its nucleotide sequence (target mRNA), function(s), diseases and disorders related thereto, and example siRNA sequences are found in the priority document patent application PCT/US2008/055563 and associated sequence listing, the contents of which are hereby incorporated by reference in their entirety.

More detail regarding HDAC and its nucleotide sequence (target mRNA), function(s), diseases and disorders related thereto, and example siRNA sequences are found in the priority document patent application PCT/US2008/055612 and associated sequence listing, the contents of which are hereby incorporated by reference in their entirety.

More detail regarding MS4A1 and its nucleotide sequence (target mRNA), function(s), diseases and disorders related thereto, and example siRNA sequences are found in the priority document patent application PCT/US2008/055622 and associated sequence listing, the contents of which are hereby incorporated by reference in their entirety.

More detail regarding CD22 and its nucleotide sequence (target mRNA), function(s), diseases and disorders related thereto, and example siRNA sequences are found in the priority document patent application PCT/US2008/055625 and associated sequence listing, the contents of which are hereby incorporated by reference in their entirety.

More detail regarding DGAT1 and DGAT2 and their nucleotide sequences (target mRNAs), functions, diseases and disorders related thereto, and example siRNA sequences are found in the priority document patent application PCT/US2008/055527 and associated sequence listing, the contents of which are hereby incorporated by reference in their entirety.

More detail regarding CD3 and its nucleotide sequence (target mRNA), function(s), diseases and disorders related thereto, and example siRNA sequences are found in the priority document patent application PCT/US2008/055533 and associated sequence listing, the contents of which are hereby incorporated by reference in their entirety.

More detail regarding PCSK9 and its nucleotide sequence (target mRNA), function(s), diseases and disorders related thereto, and example siRNA sequences are found in the priority document patent application PCT/US2008/055554 and associated sequence listing, the contents of which are hereby incorporated by reference in their entirety.

More detail regarding MET and its nucleotide sequence (target mRNA), function(s), diseases and disorders related thereto, and example siRNA sequences are found in the priority document patent application PCT/US2008/055511 and associated sequence listing, the contents of which are hereby incorporated by reference in their entirety.

More detail regarding CTNNB1 and its nucleotide sequence (target mRNA), function(s), diseases and disorders related thereto, and example siRNA sequences are found in the priority document patent application PCT/US2008/055532 and associated sequence listing, the contents of which are hereby incorporated by reference in their entirety.

More detail regarding ID and its nucleotide sequence (target mRNA), function(s), diseases and disorders related thereto, and example siRNA sequences are found in the priority document patent application PCT/US2008/055516 and associated sequence listing, the contents of which are hereby incorporated by reference in their entirety.

More detail regarding PTPN1 and its nucleotide sequence (target mRNA), function(s), diseases and disorders related thereto, and example siRNA sequences are found in the priority document patent application PCT/US2008/055551 and associated sequence listing, the contents of which are hereby incorporated by reference in their entirety.

More detail regarding TIE1 and TEK and their nucleotide sequences (target mRNAs), functions, diseases and disorders related thereto, and example siRNA sequences are found in the priority document patent application PCT/US2008/055519 and associated sequence listing, the contents of which are hereby incorporated by reference in their entirety.

More detail regarding FGFR and its nucleotide sequence (target mRNA), function(s), diseases and disorders related thereto, and example siRNA sequences are found in the priority document patent application PCT/US2008/055542 and associated sequence listing, the contents of which are hereby incorporated by reference in their entirety.

More detail regarding MAPK3 and its nucleotide sequence (target mRNA), function(s), diseases and disorders related thereto, and example siRNA sequences are found in the priority document patent application PCT/US2008/055526 and associated sequence listing, the contents of which are hereby incorporated by reference in their entirety.

More detail regarding survivin (BIRC5) and its nucleotide sequence (target mRNA), function(s), diseases and disorders related thereto, and example siRNA sequences are found in patent application PCT/US2009/52878 and associated sequence listing, the contents of which are hereby incorporated by reference in their entirety.

More detail regarding PLK1, PLK2, and PLK3 and their nucleotide sequences (target mRNAs), functions, diseases and disorders related thereto, and example siRNA sequences are found in patent application PCT/US2009/52888 and associated sequence listing, the contents of which are hereby incorporated by reference in their entirety.

As used herein, reference to target mRNA or target RNA sequences or sense strands means an RNA isoform of a nucleotide sequence of a gene identified herein, as well as variants and homologs having at least 80% or more identity with the target mRNA.

The “percent identity” between two or more nucleic acid sequences is a function of the number of identical positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., BLASTN, see www.ncbi.nlm.nih.gov/BLAST; see also Altschul et al., J. Mol. Biol. 215:403-410, 1990).

In one aspect, the instant disclosure provides an mdRNA molecule, comprising a first strand that is complementary to a target mRNA, and a second strand and a third strand that are each complementary to non-overlapping regions of the first strand, wherein the second strand and third strands can anneal with the first strand to form at least two double-stranded regions spaced apart by up to 10 nucleotides and thereby forming a gap between the second and third strands, and wherein (a) the mdRNA molecule optionally has at least one double-stranded region of 5 base pairs to 13 base pairs, or (b) the combined double-stranded regions total about 15 base pairs to about 40 base pairs and the mdRNA molecule optionally has blunt ends; wherein at least one pyrimidine of the mdRNA is substituted with a pyrimidine nucleoside according to Formula I or II:

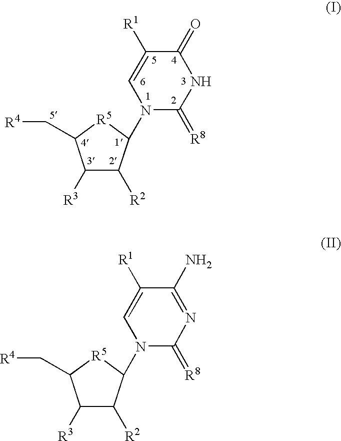

wherein

R¹ and R² are each independently a —H, —OH, —OCH₃, —OCH₂OCH₂CH₃, —OCH₂CH₂OCH₃, halogen, substituted or unsubstituted C₁-C₁₀ alkyl, alkoxy, alkoxyalkyl, hydroxyalkyl, carboxyalkyl, alkylsulfonylamino, aminoalkyl, dialkylamino, alkylaminoalkyl, dialkylaminoalkyl, haloalkyl, trifluoromethyl, cycloalkyl, (cycloalkyl)alkyl, substituted or unsubstituted C₂-C₁₀ alkenyl, substituted or unsubstituted —O-allyl, —O—CH₂CH═CH₂, —O—CH═CHCH₃, substituted or unsubstituted C₂-C₁₀ alkynyl, carbamoyl, carbamyl, carboxy, carbonylamino, substituted or unsubstituted aryl, substituted or unsubstituted aralkyl, —NH₂, —NO₂, —C≡N, or heterocyclo group;

R³ and R⁴ are each independently a hydroxyl, a protected hydroxyl, a phosphate, or an internucleoside linking group; and

R⁵ and R⁸ are each independently O or S.

In certain embodiments, at least one nucleoside is according to Formula I in which R¹ is methyl and R² is —OH, or R¹ is methyl, R² is —OH, and R⁸ is S.

In other embodiments, the internucleoside linking group covalently links from about 5 to about 40 nucleosides.

In some embodiments, the gap comprises at least one unpaired nucleotide in the first strand positioned between the double-stranded regions formed by the second and third strands when annealed to the first strand, or the gap is a nick. In certain embodiments, the nick or gap is located 10 nucleotides from the 5′-end of the first (antisense) strand or at the Argonaute cleavage site. In another embodiment, the meroduplex nick or gap is positioned such that the thermal stability is maximized for the first and second strand duplex and for the first and third strand duplex as compared to the thermal stability of such meroduplexes having a nick or gap in a different position.

In still another aspect, the instant disclosure provides an mdRNA molecule, comprising a first strand that is complementary to a target mRNA, and a second strand and a third strand that are each complementary to non-overlapping regions of the first strand, wherein the second strand and third strands can anneal with the first strand to form at least two double-stranded regions spaced apart by up to 10 nucleotides and thereby forming a gap between the second and third strands, and wherein the mdRNA molecule optionally includes at least one double-stranded region of 5 base pairs to 13 base pairs. In a further aspect, the instant disclosure provides an mdRNA molecule having a first strand that is complementary to a target mRNA, and a second strand and a third strand that are each complementary to non-overlapping regions of the first strand, wherein the second strand and third strands can anneal with the first strand to form at least two double-stranded regions spaced apart by up to 10 nucleotides and thereby forming a gap between the second and third strands, and wherein the combined double-stranded regions total about 15 base pairs to about 40 base pairs and the mdRNA molecule optionally has blunt ends. In some embodiments, the gap comprises at least one unpaired nucleotide in the first strand positioned between the double-stranded regions formed by the second and third strands when annealed to the first strand, or the gap is a nick. In certain embodiments, the nick or gap is located 10 nucleotides from the 5′-end of the first (antisense) strand or at the Argonaute cleavage site. In another embodiment, the meroduplex nick or gap is positioned such that the thermal stability is maximized for the first and second strand duplex and for the first and third strand duplex as compared to the thermal stability of such meroduplexes having a nick or gap in a different position.

As provided herein, any of the aspects or embodiments disclosed herein would be useful in treating a target mRNA-associated diseases or disorders, such as cancer, a metabolic disease and/or inflammatory disease.

In some embodiments, the dsRNA comprises at least three strands in which the first strand comprises about 5 nucleotides to about 40 nucleotides, and the second and third strands include each, individually, about 5 nucleotides to about 20 nucleotides, wherein the combined length of the second and third strands is about 15 nucleotides to about 40 nucleotides. In other embodiments, the dsRNA comprises at least two strands in which the first strand comprises about 15 nucleotides to about 24 nucleotides or about 25 nucleotides to about 40 nucleotides. In yet other embodiments, the first strand comprises about 15 to about 24 nucleotides or about 25 nucleotides to about 40 nucleotides and is complementary to at least 15, 16, 17, 18, 19, 20, 21, 22, 23, 24, 25, 26, 27, 28, 29, 30, 31, 32, 33, 34, 35, 36, 37, 38, 39, or 40 contiguous nucleotides of a human target mRNA. In alternative embodiments, the first strand comprises about 15 to about 24 nucleotides or about 25 nucleotides to about 40 nucleotides and is at least 70%, 75%, 80%, 85%, 90%, 95%, 96%, 97%, 98%, or 99% identical to a sequence that is complementary to at least 15, 16, 17, 18, 19, 20, 21, 22, 23, 24, 25, 26, 27, 28, 29, 30, 31, 32, 33, 34, 35, 36, 37, 38, 39, or 40 contiguous nucleotides of a human target mRNA.

In further embodiments, the first strand will be complementary to a second strand, to a second and third strand, or to a plurality of strands. The first strand and its complements will be able to form dsRNA and mdRNA molecules of this disclosure, but only about 19 to about 25 nucleotides of the first strand comprise a sequence complementary to a target mRNA. For example, a Dicer substrate dsRNA can have about 25 nucleotides to about 40 nucleotides, but with only 19 nucleotides of the antisense (first) strand being complementary to a target mRNA. In further embodiments, the first strand having complementarity to a target mRNA in about 19 nucleotides to about 25 nucleotides will have one, two, or three mismatches with a target mRNA, or the first strand of 19 nucleotides to about 25 nucleotides, that for example activates or is capable of loading into RISC, will have at least 80% identity with the corresponding nucleotides found in a target mRNA.

Certain illustrative dsRNA molecules, which can be used to design mdRNA or dsRNA molecules and can optionally include substitutions or modifications as described herein are provided in the Sequence Listings as attached herewith, which is herein incorporated by reference (text file named “07-R-US-CIP_Sequence_Listing”). In addition, the content of Table B as disclosed in U.S. Provisional Patent Application No. 60/934,930 (filed Mar. 16, 2007), which was submitted with that application as a separate text file named “Table_B_Human_RefSeq_Accession_Numbers.txt” (created Mar. 16, 2007 and having a size of 3,604 kilobytes), is incorporated herein by reference in its entirety.

Substituting and Modifying Target dsRNA Molecules

The introduction of substituted and modified nucleotides into mdRNA and dsRNA molecules of this disclosure provides a powerful tool in overcoming potential limitations of in vivo stability and bioavailability inherent to native RNA molecules (i.e., having standard nucleotides) that are exogenously delivered. For example, the use of dsRNA molecules of this disclosure can enable a lower dose of a particular nucleic acid molecule for a given therapeutic effect (e.g., reducing or silencing target gene expression) since dsRNA molecules of this disclosure tend to have a longer half-life in serum. Furthermore, certain substitutions and modifications can improve the bioavailability of dsRNA by targeting particular cells or tissues or improving cellular uptake of the dsRNA molecules. Therefore, even if the activity of a dsRNA molecule of this disclosure is reduced as compared to a native RNA molecule, the overall activity of the substituted or modified dsRNA molecule can be greater than that of the native RNA molecule due to improved stability or delivery of the molecule. Unlike native unmodified dsRNA, substituted and modified dsRNA can also minimize the possibility of activating the interferon response in, for example, humans.

In certain embodiments, a dsRNA molecule of this disclosure has at least one uridine, at least three uridines, or each and every uridine (i.e., all uridines) of the first (antisense) strand of the dsRNA substituted or replaced with 5-methyluridine or 2-thioribothymidine. In a related embodiment, the dsRNA molecule or analog thereof of this disclosure has at least one uridine, at least three uridines, or each and every uridine of the second (sense) strand of the dsRNA substituted or replaced with 5-methyluridine or 2-thioribothymidine. In a related embodiment, the dsRNA molecule or analog thereof of this disclosure has at least one uridine, at least three uridines, or each and every uridine of the third (sense) strand of the dsRNA substituted or replaced with 5-methyluridine or 2-thioribothymidine. In still another embodiment, the dsRNA molecule or analog thereof of this disclosure has at least one uridine, at least three uridines, or each and every uridine of both the first (antisense) and second (sense) strands; of both the first (antisense) and third (sense) strands; of both the second (sense) and third (sense) strands; or of all of the first (antisense), second (sense) and third (sense) strands of the dsRNA substituted or replaced with 5-methyluridine or 2-thioribothymidine. In some embodiments, the double-stranded region of a dsRNA molecule has at least three 5-methyluridines or 2-thioribothymidines. In certain embodiments, dsRNA molecules comprise ribonucleotides at about 5% to about 95% of the nucleotide positions in one strand, both strands, or any combination thereof.

In further embodiments, a dsRNA molecule that decreases expression of a target gene by RNAi according to the instant disclosure further comprises one or more natural or synthetic non-standard nucleoside. In related embodiments, the non-standard nucleoside is one or more deoxyuridine, locked nucleic acid (LNA) molecule, a modified base (e.g., 5-methyluridine), a universal-binding nucleotide, a 2′-O-methyl nucleotide, a modified internucleoside linkage (e.g., phosphorothioate), a G clamp, or any combination thereof. In certain embodiments, the universal-binding nucleotide can be C-phenyl, C-naphthyl, inosine, azole carboxamide, 1-β-D-ribofuranosyl-4-nitroindole, ribofuranosyl-5-nitroindole, 1-β-D-ribofuranosyl-6-nitroindole, or 1-β-D-ribofuranosyl-3-nitropyrrole.

Substituted or modified nucleotides present in dsRNA molecules, preferably in the sense or antisense strand, but also optionally in both the antisense and sense strands, comprise modified or substituted nucleotides according to this disclosure having properties or characteristics similar to natural or standard ribonucleotides. For example, this disclosure features dsRNA molecules including nucleotides having a Northern conformation (e.g., Northern pseudorotation cycle; see, e.g., Saenger, Principles of Nucleic Acid Structure, Springer-Verlag ed., 1984). As such, chemically modified nucleotides present in dsRNA molecules of this disclosure, preferably in the antisense strand, but also optionally in the sense or both the antisense and sense strands, are resistant to nuclease degradation while at the same time maintaining the capacity to mediate RNAi. Exemplary nucleotides having a Northern configuration include locked nucleic acid (LNA) nucleotides (e.g., 2′-O, 4′-C-methylene-(D-ribofuranosyl) nucleotides), 2′-methoxyethyl (MOE) nucleotides, 2′-methyl-thio-ethyl, 2′-deoxy-2′-fluoro nucleotides, 2′-deoxy-2′-chloro nucleotides, 2′-azido nucleotides, 5-methyluridines, or 2′-O-methyl nucleotides. In certain embodiments, the LNA is a 5-methyluridine LNA or 2-thio-5-methyluridine LNA. In any of these embodiments, one or more substituted or modified nucleotides can be a G clamp (e.g., a cytosine analog that forms an additional hydrogen bond to guanine, such as 9-(aminoethoxy)phenoxazine; see, e.g., Lin and Mateucci, J. Am. Chem. Soc. 120:8531, 1998).

As described herein, the first and one or more second strands of a dsRNA molecule or analog thereof provided by this disclosure can anneal or hybridize together (i.e., due to complementarity between the strands) to form at least one double-stranded region having a length of about 4 to about 10 base pairs, about 5 to about 13 base pairs, or about 15 to about 40 base pairs. In some embodiments, the dsRNA has at least one double-stranded region ranging in length from about 15 to about 24 base pairs or about 19 to about 23 base pairs. In other embodiments, the dsRNA has at least one double-stranded region ranging in length from about 26 to about 40 base pairs or about 27 to about 30 base pairs or about 30 to about 35 base pairs. In other embodiments, the two or more strands of a dsRNA molecule of this disclosure may optionally be covalently linked together by nucleotide or non-nucleotide linker molecules.

In certain embodiments, the dsRNA molecule or analog thereof comprises an overhang of one to four nucleotides on one or both 3′-ends of the dsRNA, such as an overhang comprising a deoxyribonucleotide or two deoxyribonucleotides (e.g., thymidine, adenine). In certain embodiments, the 3′-end comprising one or more deoxyribonucleotide is in an mdRNA molecule and is either in the gap, not in the gap, or any combination thereof. In some embodiments, dsRNA molecules or analogs thereof have a blunt end at one or both ends of the dsRNA. In certain embodiments, the 5′-end of the first or second strand is phosphorylated. In any of the embodiments of dsRNA molecules described herein, the 3′-terminal nucleotide overhangs can comprise ribonucleotides or deoxyribonucleotides that are chemically-modified at a nucleic acid sugar, base, or backbone. In any of the embodiments of dsRNA molecules described herein, the 3′-terminal nucleotide overhangs can comprise one or more universal base ribonucleotides. In any of the embodiments of dsRNA molecules described herein, the 3′-terminal nucleotide overhangs can comprise one or more acyclic nucleotides. In any of the embodiments of dsRNA molecules described herein, the dsRNA can further comprise a terminal phosphate group, such as a 5′-phosphate (see Martinez et al., Cell. 110:563-574, 2002; and Schwarz et al., Molec. Cell 10:537-568, 2002) or a 5′,3′-diphosphate.

As set forth herein, the terminal structure of dsRNAs of this disclosure that decrease expression of a target gene by, for example, RNAi may either have blunt ends or one or more overhangs. In certain embodiments, the overhang may be at the 3′-end or the 5′-end. The total length of dsRNAs having overhangs is expressed as the sum of the length of the paired double-stranded portion together with the overhanging nucleotides. For example, if a 19 base pair dsRNA has a two nucleotide overhang at both ends, the total length is expressed as 21-mer. Furthermore, since the overhanging sequence may have low specificity to a target gene, it is not necessarily complementary (antisense) or identical (sense) to a target gene sequence. In further embodiments, a dsRNA of this disclosure that decreases expression of a target gene by RNAi may further comprise a low molecular weight structure (e.g., a natural RNA molecule such as a tRNA, rRNA or viral RNA, or an artificial RNA molecule) at, for example, one or more overhanging portion of the dsRNA.

In further embodiments, a dsRNA molecule that decreases expression of an target gene by RNAi according to the instant disclosure further comprises a 2′-sugar substitution, such as 2′-deoxy, 2′-O-methyl, 2′-O-methoxyethyl, 2′-O-2-methoxyethyl, halogen, 2′-fluoro, 2′-O-allyl, or the like, or any combination thereof. In still further embodiments, a dsRNA molecule that decreases expression of a target gene by RNAi according to the instant disclosure further comprises a terminal cap substituent on one or both ends of the first strand or one or more second strands, such as an alkyl, abasic, deoxy abasic, glyceryl, dinucleotide, acyclic nucleotide, inverted deoxynucleotide moiety, or any combination thereof. In certain embodiments, at least one or two 5′-terminal ribonucleotides of the sense strand within the double-stranded region have a 2′-sugar substitution. In certain other embodiments, at least one or two 5′-terminal ribonucleotides of the antisense strand within the double-stranded region have a 2′-sugar substitution. In certain embodiments, at least one or two 5′-terminal ribonucleotides of the sense strand and the antisense strand within the double-stranded region have a 2′-sugar substitution.

In other embodiments, a dsRNA molecule that decreases expression of one or more target gene by RNAi according to the instant disclosure comprises one or more substitutions in the sugar backbone, including any combination of ribosyl, 2′-deoxyribosyl, a tetrofuranosyl (e.g., L-α-threofuranosyl), a hexopyranosyl (e.g., β-allopyranosyl, β-altropyranosyl, and β-glucopyranosyl), a pentopyranosyl (e.g., β-ribopyranosyl, α-lyxopyranosyl, β-xylopyranosyl, and α-arabinopyranosyl), a carbocyclic (carbon only ring) analog, a pyranose, a furanose, a morpholino, or analogs or derivatives thereof.

In yet other embodiments, a dsRNA molecule that decreases expression of a target gene (including a mRNA splice variant thereof) by RNAi according to the instant disclosure further comprises at least one modified internucleoside linkage, such as independently a phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, thionophosphoramidate, thionoalkylphosphonate, thionoalkylphosphotriester, selenophosphate, boranophosphate linkage, or any combination thereof.

A modified internucleotide linkage, as described herein, can be present in one or more strands of a dsRNA molecule of this disclosure, for example, in the sense strand, the antisense strand, both strands, or a plurality of strands (e.g., in an mdRNA). The dsRNA molecules of this disclosure can comprise one or more modified internucleotide linkages at the 3′-end, the 5′-end, or both of the 3′- and 5′-ends of the second sense strand, the third sense strand, the antisense strand or any combination of the antisense strand and one or more of the sense strands. In one embodiment, a dsRNA molecule capable of decreasing expression of a target gene (including a specific or selected mRNA splice variant thereof) by RNAi has one modified internucleotide linkage at the 3′-end, such as a phosphorothioate linkage. For example, this disclosure provides a dsRNA molecule capable of decreasing expression of a target gene by RNAi having about 1 to about 8 or more phosphorothioate internucleotide linkages in one dsRNA strand. In yet another embodiment, this disclosure provides a dsRNA molecule capable of decreasing expression of a target gene by RNAi having about 1 to about 8 or more phosphorothioate internucleotide linkages in the dsRNA strands. In other embodiments, an exemplary dsRNA molecule of this disclosure can comprise from about 1 to about 5 or more consecutive phosphorothioate internucleotide linkages at the 5′-end of the sense strand, the antisense strand, both strands, or a plurality of strands. In another example, an exemplary dsRNA molecule of this disclosure can comprise one or more pyrimidine phosphorothioate internucleotide linkages in the sense strand, the antisense strand, either strand, or a plurality of strands. In yet another example, an exemplary dsRNA molecule of this disclosure comprises one or more purine phosphorothioate internucleotide linkages in the sense strand, the antisense strand, either strand, or a plurality of strands.

Many exemplary modified nucleotide bases or analogs thereof useful in the dsRNA of the instant disclosure include 5-methylcytosine; 5-hydroxymethylcytosine; xanthine; hypoxanthine; 2-aminoadenine; 6-methyl, 2-propyl, or other alkyl derivatives of adenine and guanine; 8-substituted adenines and guanines (such as 8-aza, 8-halo, 8-amino, 8-thiol, 8-thioalkyl, 8-hydroxyl, or the like); 7-methyl, 7-deaza, and 3-deaza adenines and guanines; 2-thiouracil; 2-thiothymine; 2-thiocytosine; 5-methyl, 5-propynyl, 5-halo (such as 5-bromo or 5-fluoro), 5-trifluoromethyl, or other 5-substituted uracils and cytosines; and 6-azouracil. Further useful nucleotide bases can be found in Kurreck, Eur. J. Biochem. 270:1628, 2003; Herdewijn, Antisense Nucleic Acid Develop. 10:297, 2000; Concise Encyclopedia of Polymer Science and Engineering, pages 858-859, Kroschwitz, J. I., ed. John Wiley & Sons, 1990; U.S. Pat. No. 3,687,808, and similar references.

Certain nucleotide base moieties are particularly useful for increasing the binding affinity of the dsRNA molecules of this disclosure to complementary targets. These include 5-substituted pyrimidines; 6-azapyrimidines; and N-2, N-6, or O-6 substituted purines (including 2-aminopropyladenine, 5-propynyluracil and 5-propynylcytosine). For example, 5-methyluridine and 5-methylcytosine substitutions are known to increase nucleic acid duplex stability, which can be combined with 2′-sugar modifications (such as 2′-methoxy or 2′-methoxyethyl) or internucleotide linkages (e.g., phosphorothioate) that provide nuclease resistance to the modified or substituted dsRNA.

In another aspect of the instant disclosure, there is provided a dsRNA that decreases expression of a target gene, comprising a first strand that is complementary to a target mRNA and a second strand that is complementary to the first strand, wherein the first and second strands form a double-stranded region of about 15 to about 40 base pairs; wherein at least one pyrimidine of the dsRNA is substituted with a pyrimidine nucleoside according to Formula I or II:

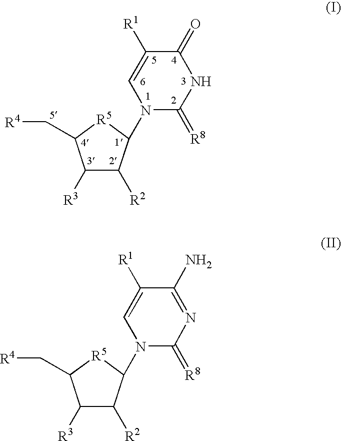

wherein

R¹ and R² are each independently a —H, —OH, —OCH₃, —OCH₂OCH₂CH₃, —OCH₂CH₂OCH₃, halogen, substituted or unsubstituted C₁-C₁₀ alkyl, alkoxy, alkoxyalkyl, hydroxyalkyl, carboxyalkyl, alkylsulfonylamino, aminoalkyl, dialkylamino, alkylaminoalkyl, dialkylaminoalkyl, haloalkyl, trifluoromethyl, cycloalkyl, (cycloalkyl)alkyl, substituted or unsubstituted C₂-C₁₀ alkenyl, substituted or unsubstituted —O-allyl, —O—CH₂CH═CH₂, —O—CH═CHCH₃, substituted or unsubstituted C₂-C₁₀ alkynyl, carbamoyl, carbamyl, carboxy, carbonylamino, substituted or unsubstituted aryl, substituted or unsubstituted aralkyl, —NH₂, —NO₂, —C≡N, or heterocyclo group;

R³ and R⁴ are each independently a hydroxyl, a protected hydroxyl, or an internucleoside linking group; and

R⁵ and R⁸ are each independently O or S.

In certain embodiments, at least one nucleoside is according to Formula I in which R¹ is methyl and R² is —OH, or R¹ is methyl, R² is —OH, and R⁸ is S. In other embodiments, the internucleoside linking group covalently links from about 2 to about 40 nucleosides.

In certain embodiments, the first and one or more second strands of a dsRNA, which decreases expression of a target gene by RNAi and has at least one pyrimidine substituted with a pyrimidine nucleoside according to Formula I or II, can anneal or hybridize together (i.e., due to complementarity between the strands) to form at least one double-stranded region having a length or a combined length of about 15 to about 40 base pairs. In some embodiments, the dsRNA has at least one double-stranded region ranging in length from about 4 base pairs to about 10 base pairs or about 5 to about 13 base pairs or about 15 to about 25 base pairs or about 19 to about 23 base pairs. In other embodiments, the dsRNA has at least one double-stranded region ranging in length from about 26 to about 40 base pairs or about 27 to about 30 base pairs or about 30 to about 35 base pairs. In certain embodiments, the dsRNA molecule or analog thereof has an overhang of one to four nucleotides on one or both 3′-ends, such as an overhang comprising a deoxyribonucleotide or two deoxyribonucleotides (e.g., thymidine). In some embodiments, dsRNA molecule or analog thereof has a blunt end at one or both ends of the dsRNA. In certain embodiments, the 5′-end of the first or second strand is phosphorylated.

In certain embodiments, at least one R¹ is a C₁-C₅ alkyl, such as methyl or ethyl. Within other exemplary embodiments of this disclosure, compounds of Formula I are a 5-alkyluridine (i.e., R¹ is alkyl, R² is —OH, and R³, R⁴, and R⁵ are as defined herein) or compounds of Formula II are a 5-alkylcytidine (i.e., R¹ is alkyl, R² is —OH, and R³, R⁴, and R⁵ are as defined herein). In related embodiments, the 5-alkyluridine is a 5-methyluridine (also referred to as ribothymidine or T^{r}—i.e., R¹ is methyl and R² is —OH), and the 5-alkylcytidine is a 5-methylcytidine. In other embodiments, at least one, at least three, or all uridines of the first strand of the dsRNA are replaced with 5-methyluridine, or at least one, at least three, or all uridines of the second strand of the dsRNA are replaced with 5-methyluridine, or any combination thereof (e.g., such changes are made on more than one strand).

In certain embodiments, at least one pyrimidine nucleoside of Formula I or Formula II has an R⁵ that is S or R⁸ that is S. In further embodiments, at least one pyrimidine nucleoside of the dsRNA is a locked nucleic acid (LNA) in the form of a bicyclic sugar, wherein R² is oxygen, and the 2′-O and 4′-C form an oxymethylene bridge on the same ribose ring. In a related embodiment, the LNA comprises a base substitution, such as a 5-methyluridine LNA or 2-thio-5-methyluridine LNA. In other embodiments, at least one, at least three, or all uridines of the first strand of the dsRNA are replaced with 5-methyluridine or 2-thioribothymidine or 5-methyluridine LNA or 2-thio-5-methyluridine LNA, or at least one, at least three, or all uridines of the second strand of the dsRNA are replaced with 5-methyluridine, 2-thioribothymidine, 5-methyluridine LNA, 2-thio-5-methyluridine LNA, or any combination thereof (e.g., such changes are made on both strands, or some substitutions include 5-methyluridine only, 2-thioribothymidine only, 5-methyluridine LNA only, 2-thio-5-methyluridine LNA only, or one or more 5-methyluridine or 2-thioribothymidine with one or more 5-methyluridine LNA or 2-thio-5-methyluridine LNA).

In further embodiments, a ribose of the pyrimidine nucleoside or the internucleoside linkage can be optionally modified. For example, compounds of Formula I or II are provided wherein R² is alkoxy, such as a 2′-O-methyl substitution (e.g., which may be in addition to a 5-alkyluridine or a 5-alkylcytidine, respectively). In certain embodiments, R² is selected from 2′-O—(C₁-C₅) alkyl, 2′-O-methyl, 2′-OCH₂OCH₂CH₃, 2′-OCH₂CH₂OCH₃, 2′-O-allyl, or 2′-fluoro.

In further embodiments, one or more of the pyrimidine nucleosides are according to Formula I in which R¹ is methyl and R² is a 2′-O—(C₁-C₅) alkyl (e.g., 2′-O-methyl), or in which R¹ is methyl, R² is a 2′O—(C₁-C₅) alkyl (e.g., 2′O-methyl), and R² is S, or any combination thereof. In other embodiments, one or more, or at least two, pyrimidine nucleosides according to Formula I or II have an R² that is not —H or —OH and is incorporated at a 3′-end or 5′-end and not within the gap of one or more strands within the double-stranded region of the dsRNA molecule.

In further embodiments, a dsRNA molecule or analog thereof comprising a pyrimidine nucleoside according to Formula I or Formula II in which R² is not —H or —OH and an overhang, further comprises at least two of pyrimidine nucleosides that are incorporated either at a 3′-end or a 5′-end or both of one strand or two strands within the double-stranded region of the dsRNA molecule. In a related embodiment, at least one of the at least two pyrimidine nucleosides in which R² is not —H or —OH is located at a 3′-end or a 5′-end within the double-stranded region of at least one strand of the dsRNA molecule, and wherein at least one of the at least two pyrimidine nucleosides in which R² is not —H or —OH is located internally within a strand of the dsRNA molecule.

In still further embodiments, a dsRNA molecule or analog thereof that has an overhang has a first of the two or more pyrimidine nucleosides in which R² is not —H or —OH that is incorporated at a 5′-end within the double-stranded region of the sense strand of the dsRNA molecule and a second of the two or more pyrimidine nucleosides is incorporated at a 5′-end within the double-stranded region of the antisense strand of the dsRNA molecule. In any of these embodiments, one or more substituted or modified nucleotides can be a G clamp (e.g., a cytosine analog that forms an additional hydrogen bond to guanine, such as 9-(aminoethoxy)phenoxazine; see, e.g., Lin and Mateucci, 1998). In any of these embodiments, provided the one or more pyrimidine nucleosides are not within the gap.

In yet other embodiments, a dsRNA molecule or analog thereof of Formula I or II according to the instant disclosure that has an overhang that comprises four or more independent pyrimidine nucleosides or four or more independent pyrimidine nucleosides in which R² is not —H or —OH, wherein (a) a first pyrimidine nucleoside is incorporated into a 3′-end within the double-stranded region of the sense (second) strand of the dsRNA, (b) a second pyrimidine nucleoside is incorporated into a 5′-end within the double-stranded region of the sense (second) strand, (c) a third pyrimidine nucleoside is incorporated into a 3′-end within the double-stranded region of the antisense (first) strand of the dsRNA, and (d) a fourth pyrimidine nucleoside is incorporated into a 5′-end within the double-stranded region of the antisense (first) strand. In any of these embodiments, provided the one or more pyrimidine nucleosides are not within the gap.

In further embodiments, a dsRNA molecule or analog thereof comprising a pyrimidine nucleoside according to Formula I or Formula II in which R² is not —H or —OH and is blunt-ended, further comprises at least two of pyrimidine nucleosides that are incorporated either at a 3′-end or a 5′-end or both of one strand or two strands of the dsRNA molecule. In a related embodiment, at least one of the at least two pyrimidine nucleosides in which R² is not —H or —OH is located at a 3′-end or a 5′-end of at least one strand of the dsRNA molecule, and wherein at least one of the at least two pyrimidine nucleosides in which R² is not —H or —OH is located internally within a strand of the dsRNA molecule. In still further embodiments, a dsRNA molecule or analog thereof that is blunt-ended has a first of the two or more pyrimidine nucleosides in which R² is not —H or —OH that is incorporated at a 5′-end of the sense strand of the dsRNA molecule and a second of the two or more pyrimidine nucleosides is incorporated at a 5′-end of the antisense strand of the dsRNA molecule. In any of these embodiments, provided the one or more pyrimidine nucleosides are not within the gap.

In yet other embodiments, a dsRNA molecule comprising a pyrimidine nucleoside according to Formula I or Formula II and that is blunt-ended comprises four or more independent pyrimidine nucleosides or four or more independent pyrimidine nucleosides in which R² is not —H or —OH, wherein (a) a first pyrimidine nucleoside is incorporated into a 3′-end within the double-stranded region of the sense (second) strand of the dsRNA, (b) a second pyrimidine nucleoside is incorporated into a 5′-end within the double-stranded region of the sense (second) strand, (c) a third pyrimidine nucleoside is incorporated into a 3′-end within the double-stranded region of the antisense (first) strand of the dsRNA, and (d) a fourth pyrimidine nucleoside is incorporated into a 5′-end within the double-stranded region of the antisense (first) strand. In any of these embodiments, provided the one or more pyrimidine nucleosides are not within the gap.

In still further embodiments, a dsRNA molecule or analog thereof of Formula I or II according to the instant disclosure further comprises a terminal cap substituent on one or both ends of the first strand or second strand, such as an alkyl, abasic, deoxy abasic, glyceryl, dinucleotide, acyclic nucleotide, inverted deoxynucleotide moiety, or any combination thereof. In further embodiments, one or more internucleoside linkage can be optionally modified. For example, a dsRNA molecule or analog thereof of Formula I or II according to the instant disclosure wherein at least one internucleoside linkage is modified to a phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, thionophosphoramidate, thionoalkylphosphonate, thionoalkylphosphotriester, selenophosphate, boranophosphate linkage, or any combination thereof.

In still another embodiment, a nicked or gapped dsRNA molecule (ndsRNA or gdsRNA, respectively) that decreases expression of a target gene by RNAi, comprising a first strand that is complementary to a target mRNA and two or more second strands that are complementary to the first strand, wherein the first and at least one of the second strands form a non-overlapping double-stranded region of about 5 to about 13 base pairs. Any of the substitutions or modifications described herein is contemplated within this embodiment as well.

In another exemplary of this disclosure, the dsRNAs comprise at least two or more substituted pyrimidine nucleosides can each be independently selected wherein R¹ comprises any chemical modification or substitution as contemplated herein, for example an alkyl (e.g., methyl), halogen, hydroxy, alkoxy, nitro, amino, trifluoromethyl, cycloalkyl, (cycloalkyl)alkyl, alkanoyl, alkanoyloxy, aryl, aroyl, aralkyl, nitrile, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, carbonyl, alkanoylamino, carbamoyl, carbonylamino, alkylsulfonylamino, or heterocyclo group. When two or more modified ribonucleotides are present, each modified ribonucleotide can be independently modified to have the same, or different, modification or substitution at R¹ or R².

In other detailed embodiments, one or more substituted pyrimidine nucleosides according to Formula I or II can be located at any ribonucleotide position, or any combination of ribonucleotide positions, on either or both of the sense and antisense strands of a dsRNA molecule of this disclosure, including at one or more multiple terminal positions as noted above, or at any one or combination of multiple non-terminal (“internal”) positions. In this regard, each of the sense and antisense strands can incorporate about 1 to about 6 or more of the substituted pyrimidine nucleosides.

In certain embodiments, when two or more substituted pyrimidine nucleosides are incorporated within a dsRNA of this disclosure, at least one of the substituted pyrimidine nucleosides will be at a 3′- or 5′-end of one or both strands, and in certain embodiments at least one of the substituted pyrimidine nucleosides will be at a 5′-end of one or both strands. In other embodiments, the substituted pyrimidine nucleosides are located at a position corresponding to a position of a pyrimidine in an unmodified dsRNA that is constructed as a homologous sequence for targeting a cognate mRNA, as described herein.

In addition, the terminal structure of the dsRNAs of this disclosure may have a stem-loop structure in which ends of one side of the dsRNA molecule are connected by a linker nucleic acid, e.g., a linker RNA. The length of the double-stranded region (stem-loop portion) can be, for example, about 15 to about 49 bp, about 15 to about 35 bp, or about 21 to about 30 by long. Alternatively, the length of the double-stranded region that is a final transcription product of dsRNAs to be expressed in a target cell may be, for example, approximately about 15 to about 49 bp, about 15 to about 35 bp, or about 21 to about 30 by long. When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, for example, to construct the linker portion to include introns so that the introns are excised during processing of a precursor RNA into mature RNA, thereby allowing pairing of the stem portion. In the case of a stem-loop dsRNA, either end (head or tail) of RNA with no loop structure may have a low molecular weight RNA. As described above, these low molecular weight RNAs may include a natural RNA molecule, such as tRNA, rRNA or viral RNA, or an artificial RNA molecule.

A dsRNA molecule may be comprised of a circular nucleic acid molecule, wherein the dsRNA is about 38 to about 70 nucleotides in length having from about 18 to about 23 base pairs (e.g., about 19 to about 21 bp) wherein the circular oligonucleotide forms a dumbbell shaped structure having about 19 base pairs and two loops. In certain embodiments, a circular dsRNA molecule contains two loop motifs wherein one or both loop portions of the dsRNA molecule is biodegradable. For example, a circular dsRNA molecule of this disclosure is designed such that degradation of the loop portions of the dsRNA molecule in vivo can generate a dsRNA molecule with 3′-terminal overhangs, such as 3′-terminal nucleotide overhangs comprising from about 1 to about 4 (unpaired) nucleotides.

Substituting or modifying nucleosides of a dsRNA according to this disclosure can result in increased resistance to enzymatic degradation, such as exonucleolytic degradation, including 5′-exonucleolytic or 3′-exonucleolytic degradation. As such, in some embodiments, the dsRNAs described herein will exhibit significant resistance to enzymatic degradation compared to a corresponding dsRNA having standard nucleotides, and will thereby possess greater stability, increased half-life, and greater bioavailability in physiological environments (e.g., when introduced into a eukaryotic target cell). In addition to increasing resistance of the substituted or modified dsRNAs to exonucleolytic degradation, the incorporation of one or more pyrimidine nucleosides according to Formula I or II will render dsRNAs more resistant to other enzymatic or chemical degradation processes and thus more stable and bioavailable than otherwise identical dsRNAs that do not include the substitutions or modifications. In related aspects of this disclosure, dsRNA substitutions or modifications described herein will often improve stability of a modified dsRNA for use within research, diagnostic and treatment methods wherein the modified dsRNA is contacted with a biological sample, for example, a mammalian cell, intracellular compartment, serum or other extracellular fluid, tissue, or other in vitro or in vivo physiological compartment or environment. In one embodiment, diagnosis is performed on an isolated biological sample. In another embodiment, the diagnostic method is performed in vitro. In a further embodiment, the diagnostic method is not performed (directly) on a human or animal body.

In addition to increasing stability of substituted or modified dsRNAs, incorporation of one or more pyrimidine nucleosides according to Formula I or II in a dsRNA designed for gene silencing can provide additional desired functional results, including increasing a melting point of a substituted or modified dsRNA compared to a corresponding unmodified dsRNA. In another aspect of this disclosure, certain substitutions or modifications of dsRNAs described herein can reduce “off-target effects” of the substituted or modified dsRNA molecules when they are contacted with a biological sample (e.g., when introduced into a target eukaryotic cell having specific, and non-specific mRNA species present as potential specific and non-specific targets). In yet another aspect of this disclosure, the dsRNA substitutions or modifications described herein can reduce interferon activation by the dsRNA molecule when the dsRNA is contacted with a biological sample, e.g., when introduced into a eukaryotic cell.

In further embodiments, dsRNAs of this disclosure can comprise one or more sense (second) strand that is homologous or corresponds to a sequence of a target gene and an antisense (first) strand that is complementary to the sense strand and a sequence of the target gene. In exemplary embodiments, at least one strand of the dsRNA incorporates one or more pyrimidines substituted according to Formula I or II (e.g., wherein the pyrimidine is one or more 5-methyluridines or 2-thioribothymidines, the ribose is modified to incorporate one or more 2′-O-methyl substitutions, or any combination thereof). These and other multiple substitutions or modifications according to Formula I or II can be introduced into one or more pyrimidines, or into any combination and up to all pyrimidines present in one or more strands of a dsRNA of the instant disclosure, so long as the dsRNA has or retains RNAi activity similar to or better than the activity of an unmodified dsRNA.

In any of the embodiments described herein, the dsRNA may include multiple modifications. For example, a dsRNA having at least one ribothymidine or 2′-O-methyl-5-methyluridine may further comprise at least one LNA, 2′-methoxy, 2′-fluoro, 2′-deoxy, phosphorothioate linkage, an inverted base terminal cap, or any combination thereof. In certain embodiments, a dsRNA will have from one to all ribothymidines and have up to 75% LNA. In other embodiments, a dsRNA will have from one to all ribothymidines and have up to 75% 2′-methoxy (e.g., not at the Argonaute cleavage site). In still other embodiments, a dsRNA will have from one to all ribothymidines and have up to 100% 2′-fluoro. In further embodiments, a dsRNA will have from one to all ribothymidines and have up to 75% 2′-deoxy. In further embodiments, a dsRNA will have up to 75% LNA and have up to 75% 2′-methoxy. In still other embodiments, a dsRNA will have up to 75% LNA and have up to 100% 2′-fluoro. In further embodiments, a dsRNA will have up to 75% LNA and have up to 75% 2′-deoxy. In other embodiments, a dsRNA will have up to 75% 2′-methoxy and have up to 100% 2′-fluoro. In more embodiments, a dsRNA will have up to 75% 2′-methoxy and have up to 75% 2′-deoxy. In further embodiments, a dsRNA will have up to 100% 2′-fluoro and have up to 75% 2′-deoxy.

In further multiple modification embodiments, a dsRNA will have from one to all ribothymidines, up to 75% LNA, and up to 75% 2′-methoxy. In still further embodiments, a dsRNA will have from one to all ribothymidines, up to 75% LNA, and up to 100% 2′-fluoro. In further embodiments, a dsRNA will have from one to all ribothymidines, up to 75% LNA, and up to about 75% 2′-deoxy. In further embodiments, a dsRNA will have from one to all ribothymidines, up to 75% 2′-methoxy, and up to 75% 2′-fluoro. In further embodiments, a dsRNA will have from one to all ribothymidines, up to 75% 2′-methoxy, and up to 75% 2′-deoxy. In further embodiments, a dsRNA will have from one to all ribothymidines, up to 100% 2′-fluoro, and up to 75% 2′-deoxy. In yet further embodiments, a dsRNA will have from one to all ribothymidines, up to 75% LNA substitutions, up to 75% 2′-methoxy, up to 100% 2′-fluoro, and up to 75% 2′-deoxy. In other embodiments, a dsRNA will have up to 75% LNA, up to 75% 2′-methoxy, and up to 100% 2′-fluoro. In further embodiments, a dsRNA will have up to 75% LNA, up to 75% 2′-methoxy, and up to about 75% 2′-deoxy. In further embodiments, a dsRNA will have up to 75% LNA, up to 100% 2′-fluoro, and up to 75% 2′-deoxy. In still further embodiments, a dsRNA will have up to 75% 2′-methoxy, up to 100% 2′-fluoro, and up to 75% 2′-deoxy.

In any of these exemplary methods for using multiply modified dsRNA, the dsRNA may further comprise up to 100% phosphorothioate internucleoside linkages, from one to ten or more inverted base terminal caps, or any combination thereof. Additionally, any of these dsRNA may have these multiple modifications on one strand, two strands, three strands, a plurality of strands, or all strands, or on the same or different nucleoside within a dsRNA molecule. Finally, in any of these multiple modification dsRNA, the dsRNA must have gene silencing activity.

Within certain aspects, the present disclosure provides dsRNA that decreases expression of a target gene by RNAi and compositions comprising one or more dsRNA, wherein at least one dsRNA comprises one or more universal-binding nucleotide(s) in the first, second or third position in the anti-codon of the antisense or sense strand of the dsRNA and wherein the dsRNA is capable of specifically binding to a target sequence, such as an RNA expressed by a target cell. In cases wherein the sequence of a target target RNA includes one or more single nucleotide substitutions, dsRNA comprising a universal-binding nucleotide retains its capacity to specifically bind a target RNA, thereby mediating gene silencing and, as a consequence, overcoming escape of the target gene from dsRNA-mediated gene silencing. Exemplary universal-binding nucleotides that may be suitably employed in the compositions and methods disclosed herein include inosine, 1-β-D-ribofuranosyl-5-nitroindole, or 1-β-D-ribofuranosyl-3-nitropyrrole.

In certain aspects, dsRNA disclosed herein can include between about 1 universal-binding nucleotide and about 10 universal-binding nucleotides. Within other aspects, the presently disclosed dsRNA may comprise a sense strand that is homologous to a sequence of a target gene and an antisense strand that is complementary to the sense strand, with the proviso that at least one nucleotide of the antisense or sense strand of the otherwise complementary dsRNA duplex has one or more universal-binding nucleotide.

In certain aspects, dsRNA disclosed herein comprises one or more hydroxymethyl modified nucleomonomer(s) (see chemical formulas below). Hereunder as one such example is an acyclic nucleomonomer, more preferably an acyclic monomer selected from the group consisting of monomers D, F, G, H, I, and J. Thus, the embodiments described in the first aspect with regards to hydroxymethyl modified nucleomonomers will apply for other embodiments relating to acyclic nucleomonomers.

As used herein, the terms “hydroxylmethyl substituted nucleomonomers”, “hydroxylmethyl substituted monomers”, “acyclic nucleomonomers”, “acyclic monomers”, “acyclic hydroxymethyl subsitituted nucleomoners”, “nucleobase analog monomers” may be used interchangeably throughout.

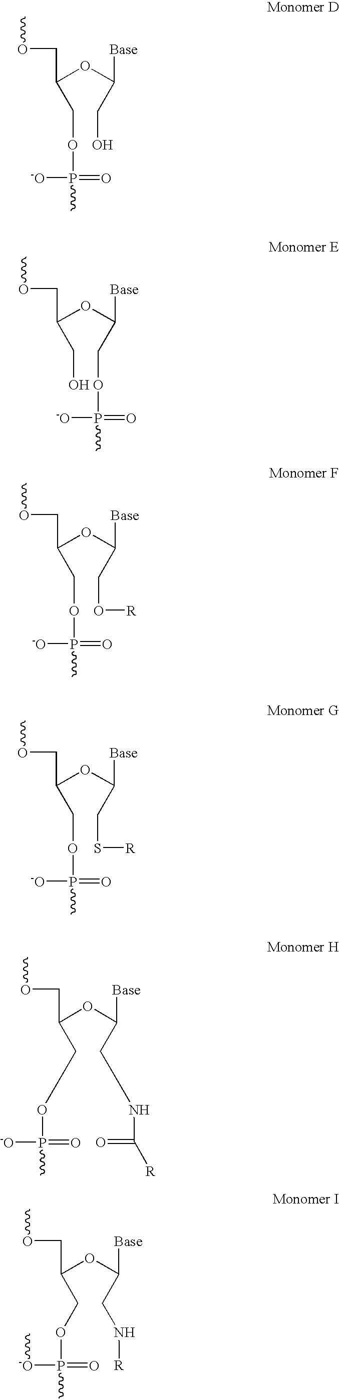

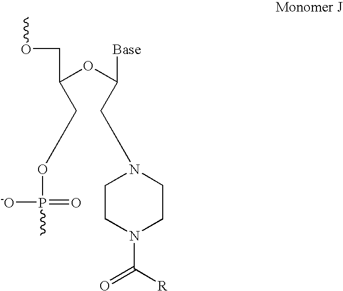

R, in the above structures, is selected from the group consisting of hydrogen, methyl group, C(1-10) alkyl, cholesterol, naturally or non-naturally occurring amino acid, sugar, vitamin, fluorophore, polyamine and fatty acid.

In certain aspects, a dsRNA having one or more hydroxymethyl modified nucleomonomer(s) has increased potency, reduced off-target effects, reduced immune stimulation, increased stability for storage, increased stability in biological media like serum, increased duration of action and/or improved pharmacokinetic properties, all relative to the native unmodified form of the dsRNA.

In certain aspects, the antisense (guide strand) of a dsRNA comprises one or more hydroxymethyl modified nucleomonomer(s). In certain aspects, the antisense of a dsRNA comprises 1, 2, 3, 4, 5, 6, 7, 8, 9 or 10 hydroxymethyl modified nucleomonomer(s). In certain aspects, the entire antisense of a dsRNA comprises hydroxymethyl modified nucleomonomer(s). In certain aspects, a hydroxymethyl modified nucleomonomer in the antisense strand is present in positions 1, 2, 3, 4, 5, 6, 7, and/or 8 wherein the positions are counted from the 5′-end of the antisense strand. In certain aspects, a hydroxymethyl modified nucleomonomer in the antisense strand is present in positions 3, 4, 5, 6, 7, and/or 8 wherein the positions are counted from the 5′-end of the antisense strand. In certain aspects, a hydroxymethyl modified nucleomonomer in the antisense strand is present in positions 7 and/or 8 wherein the positions are counted from the 5′-end of the antisense strand.

In certain aspects, a hydroxymethyl modified nucleomonomer in the antisense strand is present in positions 9, 10, 11, 12, 13, 14, 15, and/or 16 wherein the positions are counted from the 5′-end of the antisense strand. In certain aspects, a hydroxymethyl modified nucleomonomer in the antisense strand is present in positions 9, 10, and/or 11, wherein the positions are counted from the 5′-end of the antisense strand. In certain aspects, a hydroxymethyl modified nucleomonomer in the antisense strand is present in positions 1, 2, 3, 4, 5, 6, 7, 8, 9, 10, 11, 12, 13, 14, 15, 16, and/or 17, wherein the positions are counted from the 5′-end of the antisense strand. In certain aspects, a hydroxymethyl modified nucleomonomer in the antisense strand is present in positions 1, 2, 3, 4, 5, 6, 7, 8, 9 and/or 10, wherein the positions are counted from the 3′-end of the antisense strand.

In certain aspects, the sense (passenger strand) of a dsRNA comprises one or more hydroxymethyl modified nucleomonomer(s). In certain aspects, the sense (passenger strand) of a dsRNA comprises 1, 2, 3, 4, 5, 6, 7, 8, 9 or 10 hydroxymethyl modified nucleomonomer(s). In certain aspects, the entire sense (passenger strand) of a dsRNA comprises hydroxymethyl modified nucleomonomer(s). In certain aspects, a hydroxymethyl modified nucleomonomer in the sense strand is present in positions 1, 2, 3, 4, 5, 6, 7, and/or 8 wherein the positions are counted from the 5′-end of the sense strand. In certain aspects, a hydroxymethyl modified nucleomonomer in the sense strand is present in positions 3, 4, 5, 6, 7, and/or 8 wherein the positions are counted from the 5′-end of the sense strand. In certain aspects, a hydroxymethyl modified nucleomonomer in the sense strand is present in positions 7 and/or 8 wherein the positions are counted from the 5′-end of the sense strand. In certain aspects, a hydroxymethyl modified nucleomonomer in the sense strand is present in positions 9, 10, 11, 12, 13, 14, 15, and/or 16 wherein the positions are counted from the 5′-end of the sense strand. In certain aspects, a hydroxymethyl modified nucleomonomer in the sense strand is present in positions 9, 10, and/or 11, wherein the positions are counted from the 5′-end of the sense strand.). In certain aspects, a hydroxymethyl modified nucleomonomer in the sense strand is present in positions 19, 20, 21, 22, 23, 24, 25, 26, 27, 28, 29, 30, 31 and/or 32 wherein the positions are counted from the 5′-end of the sense strand. In certain aspects, a hydroxymethyl modified nucleomonomer in the sense strand is present in positions 1, 2, 3, 4, 5, 6, 7, 8, 9 and/or 10, wherein the positions are counted from the 3′-end of the sense strand.

In certain aspects, the first, second, and/or third strands of an mdRNA having a nick or gap comprises one or more hydroxymethyl modified nucleomonomer(s). In certain aspects, the first, second, and/or third strands of an mdRNA comprises 1, 2, 3, 4, 5, 6, 7, 8, 9 or 10 hydroxymethyl modified nucleomonomer(s). In certain aspects, the entire first strand of an mdRNA comprises hydroxymethyl modified nucleomonomer(s). In certain aspects, a hydroxymethyl modified nucleomonomer in the first strand is present in positions 1, 2, 3, 4, 5, 6, 7, and/or 8 wherein the positions are counted from the 5′-end of the first strand strand. In certain aspects, a hydroxymethyl modified nucleomonomer in the first strand is present in positions 9, 10, 11, 12, 13, 14, 15, and/or 16 wherein the positions are counted from the 5′-end of the first strand.

In certain aspects, the dsRNA has at least one blunt end having one or more a hydroxymethyl modified nucleomonomer(s) covalently linked to the blunt end. In certain aspects, the dsRNA has two blunt ends each having one or more a hydroxymethyl modified nucleomonomer(s) covalently linked to each blunt end. In certain aspects, a blunt end has 1, 2, 3, 4, 5, 6, 7, 8 or more hydroxymethyl modified nucleomonomers covalently linked to the blunt end. In certain aspects, a blunt end has two hydroxymethyl modified nucleomonomers covalently linked to the blunt end. In certain aspects, the one or more a hydroxymethyl modified nucleomonomer(s) are covalently linked to the 5′-end of the antisense strand. In certain aspects, the one or more a hydroxymethyl modified nucleomonomer(s) are covalently linked to the 3′-end of the antisense strand. In certain aspects, the one or more a hydroxymethyl modified nucleomonomer(s) are covalently linked to the 5′-end and the 3′-end of the antisense strand. In certain aspects, the one or more a hydroxymethyl modified nucleomonomer(s) are covalently linked to the 5′-end of the sense strand. In certain aspects, the one or more a hydroxymethyl modified nucleomonomer(s) are covalently linked to the 3′-end of the sense strand. In certain aspects, the one or more a hydroxymethyl modified nucleomonomer(s) are covalently linked to the 5′-end and the 3′-end of the sense strand. In certain aspects, the one or more a hydroxymethyl modified nucleomonomer(s) are covalently linked to the 3′-end of the sense strand and the 3′-end of the antisense strand. In certain aspects, the one or more a hydroxymethyl modified nucleomonomer(s) are covalently linked to the 5′-end of the sense strand and the 5′-end of the antisense strand. In certain aspects, the one or more a hydroxymethyl modified nucleomonomer(s) are covalently linked to the 3′-end of the sense strand and the 5′-end of the antisense strand. In certain aspects, the one or more a hydroxymethyl modified nucleomonomer(s) are covalently linked to the 5′-end of the sense strand and the 3′-end of the antisense strand.

In certain aspects, the mdRNA has at least one blunt end having one or more a hydroxymethyl modified nucleomonomer(s) covalently linked to the blunt end. In certain aspects, the mdRNA has two blunt ends each having one or more a hydroxymethyl modified nucleomonomer(s) covalently linked to each blunt end. In certain aspects, a blunt end has 1, 2, 3, 4, 5, 6, 7, 8 or more hydroxymethyl modified nucleomonomers covalently linked to the blunt end. In certain aspects, a blunt end has two hydroxymethyl modified nucleomonomers covalently linked to the blunt end. In certain aspects, the one or more a hydroxymethyl modified nucleomonomer(s) are covalently linked to the 5′-end of the antisense strand. In certain aspects, the one or more a hydroxymethyl modified nucleomonomer(s) are covalently linked to the 3′-end of the antisense strand. In certain aspects, the one or more a hydroxymethyl modified nucleomonomer(s) are covalently linked to the 5′-end and the 3′-end of the antisense strand. In certain aspects, the one or more a hydroxymethyl modified nucleomonomer(s) are covalently linked to the 5′-end of one or both of the sense strands of an mdRNA. In certain aspects, the one or more a hydroxymethyl modified nucleomonomer(s) are covalently linked to the 3′-end of one or both of the sense strands of an mdRNA. In certain aspects, the one or more a hydroxymethyl modified nucleomonomer(s) are covalently linked to the 5′-end and the 3′-end of one or both of the sense strands of an mdRNA. In certain aspects, the one or more a hydroxymethyl modified nucleomonomer(s) are covalently linked to the 3′-end of one or both of the sense strands, and the 3′-end of the antisense strand of an mdRNA. In certain aspects, the one or more a hydroxymethyl modified nucleomonomer(s) are covalently linked to the 5′-end of one or both of the sense strands, and the 5′-end of the antisense strand of an mdRNA. In certain aspects, the one or more a hydroxymethyl modified nucleomonomer(s) are covalently linked to the 3′-end of one or both of the sense strands, and the 5′-end of the antisense strand of the mdRNA. In certain aspects, the one or more a hydroxymethyl modified nucleomonomer(s) are covalently linked to the 5′-end of one or both of the sense strands, and the 3′-end of the antisense strand of an mdRNA.

In certain aspects, the dsRNA comprises hydroxymethyl substituted monomers at one or more position(s) that prevent and/or reduce dicer enzyme processing of the dsRNA compared to an unmodified form of the dsRNA.

In certain aspects, the dsRNA comprises hydroxymethyl substituted monomers at one or more position(s) that prevent and/or reduce cytokine induction by the dsRNA compared to an unmodified form of the dsRNA.

In certain aspects, the dsRNA comprises hydroxymethyl substituted monomers at one or more position(s) that improves and/or enhances the potency or target message knockdown activity of the dsRNA compared to an unmodified form of the dsRNA.

In certain aspects, the dsRNA comprises hydroxymethyl substituted monomers at one or more position(s) that prevent and/or reduce off-target effect by the dsRNA compared to an unmodified form of the dsRNA.

In certain aspects, the dsRNA comprises hydroxymethyl substituted monomers at one or more position(s) that improves and/or enhances the stability of the dsRNA in serum compared to an unmodified form of the dsRNA.

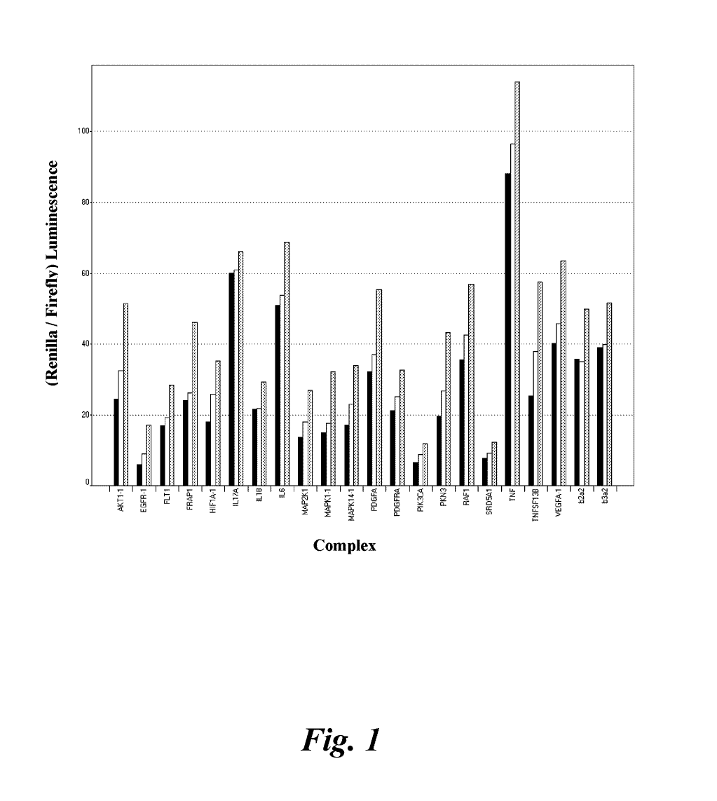

The contents of PCT patent application PCT/US2008/064417, for example FIG. 1, are hereby incorporated by reference in its entirety. Monomers disclosed in PCT/US2008/064417, for example FIG. 1, may be used in combination the dsRNA molecules disclosed herein and used in combination with any modification disclosed herein. Examples monomers include the following:

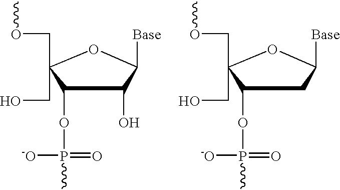

### Synthesis of Nucleic Acid Molecules

Exemplary molecules of the instant disclosure are recombinantly produced, chemically synthesized, or a combination thereof. Oligonucleotides (e.g., certain modified oligonucleotides or portions of oligonucleotides lacking ribonucleotides) are synthesized using protocols known in the art, for example as described in Caruthers et al., Methods in Enzymol. 211:3-19, 1992; Thompson et al., PCT Publication No. WO 99/54459, Wincott et al., Nucleic Acids Res. 23:2677-2684, 1995; Wincott et al., Methods Mol. Bio. 74:59, 1997; Brennan et al., Biotechnol Bioeng. 61:33-45, 1998; and Brennan, U.S. Pat. No. 6,001,311. Synthesis of RNA, including certain dsRNA molecules and analogs thereof of this disclosure, can be made using the procedure as described in Usman et al., J. Am. Chem. Soc. 109:7845, 1987; Scaringe et al., Nucleic Acids Res. 18:5433, 1990; and Wincott et al., Nucleic Acids Res. 23:2677-2684, 1995; Wincott et al., Methods Mol. Bio. 74:59, 1997.

In certain embodiments, the nucleic acid molecules of the present disclosure can be synthesized separately and joined together post-synthetically, for example, by ligation (Moore et al., Science 256:9923, 1992; Draper et al., PCT Publication No. WO 93/23569; Shabarova et al., Nucleic Acids Res. 19:4247, 1991; Bellon et al., Nucleosides & Nucleotides 16:951, 1997; Bellon et al., Bioconjugate Chem. 8:204, 1997), or by hybridization following synthesis or deprotection.

In further embodiments, dsRNAs of this disclosure that decrease expression of a target gene by RNAi can be made as single or multiple transcription products expressed by a polynucleotide vector encoding one or more dsRNAs and directing their expression within host cells. In these embodiments the double-stranded portion of a final transcription product of the dsRNAs to be expressed within the target cell can be, for example, about 5 to about 40 bp, about 15 to about 24 bp, or about 25 to about 40 by long. Within exemplary embodiments, double-stranded portions of dsRNAs, in which two or more strands pair up, are not limited to completely paired nucleotide segments, and may contain non-pairing portions due to a mismatch (the corresponding nucleotides are not complementary), bulge (lacking in the corresponding complementary nucleotide on one strand), overhang, or the like. Non-pairing portions can be contained to the extent that they do not interfere with dsRNA formation and function. In certain embodiments, a “bulge” may comprise 1 to 2 non-pairing nucleotides, and the double-stranded region of dsRNAs in which two strands pair up may contain from about 1 to 7, or about 1 to 5 bulges. In addition, “mismatch” portions contained in the double-stranded region of dsRNAs may include from about 1 to 7, or about 1 to 5 mismatches. In other embodiments, the double-stranded region of dsRNAs of this disclosure may contain both bulge and mismatched portions in the approximate numerical ranges specified herein.

A dsRNA or analog thereof of this disclosure may be further comprised of a nucleotide, non-nucleotide, or mixed nucleotide/non-nucleotide linker that joins the sense region of the dsRNA to the antisense region of the dsRNA. In one embodiment, a nucleotide linker can be a linker of more than about 2 nucleotides length up to about 10 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule wherein the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art (see, e.g., Gold et al., Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chem. 45:1628, 1999).

A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma et al., Nucleic Acids Res. 21:2585, 1993, and Biochemistry 32:1751, 1993; Durand et al., Nucleic Acids Res. 18:6353, 1990; McCurdy et al., Nucleosides & Nucleotides 10:287, 1991; Jaschke et al., Tetrahedron Lett. 34:301, 1993; Ono et al., Biochemistry 30:9914, 1991; Arnold et al., PCT Publication No. WO 89/02439; Usman et al., PCT Publication No. WO 95/06731; Dudycz et al., PCT Publication No. WO 95/11910 and Ferentz and Verdine, J. Am. Chem. Soc. 113:4000, 1991. The synthesis of a dsRNA molecule of this disclosure, which can be further modified, comprises: (a) synthesis of a first (antisense) strand and synthesis of a second (sense) strand and a third (sense) strand that are each complementary to non-overlapping regions of the first strand; and (b) annealing the first, second and third strands together under conditions suitable to obtain a dsRNA molecule. In another embodiment, synthesis of the first, second and thirdstrands of a dsRNA molecule is by solid phase oligonucleotide synthesis. In yet another embodiment, synthesis of the first, second, and third strands of a dsRNA molecule is by solid phase tandem oligonucleotide synthesis.

Chemically synthesizing nucleic acid molecules with substitutions or modifications (base, sugar, phosphate, or any combination thereof) can prevent their degradation by serum ribonucleases, which may lead to increased potency. See, e.g., Eckstein et al., PCT Publication No. WO 92/07065; Perrault et al., Nature 344:565, 1990; Pieken et al., Science 253:314, 1991; Usman and Cedergren, Trends in Biochem. Sci. 17:334, 1992; Usman et al., Nucleic Acids Symp. Ser. 31:163, 1994; Beigelman et al., J. Biol. Chem. 270:25702, 1995; Burgin et al., Biochemistry 35:14090, 1996; Burlina et al., Bioorg. Med. Chem. 5:1999, 1997; Thompson et al., Karpeisky et al., Tetrahedron Lett. 39:1131, 1998; Earnshaw and Gait, Biopolymers (Nucleic Acid Sciences) 48:39-55, 1998; Verma and Eckstein, Annu. Rev. Biochem. 67:99-134, 1998; Herdewijn, Antisense Nucleic Acid Drug Dev. 10:297, 2000; Kurreck, Eur. J. Biochem. 270:1628, 2003; Dorsett and Tuschl, Nature Rev. Drug Discov. 3:318, 2004; PCT Publication Nos. WO 91/03162; WO 93/15187; WO 97/26270; WO 98/13526; U.S. Pat. Nos. 5,334,711; 5,627,053; 5,716,824; 5,767,264; 6,300,074. Each of the above references discloses various substitutions and chemical modifications to the base, phosphate, or sugar moieties of nucleic acid molecules, which can be used in the dsRNAs described herein. For example, oligonucleotides can be modified at the sugar moiety to enhance stability or prolong biological activity by increasing nuclease resistance. Representative sugar modifications include 2′-amino, 2′-C-allyl, 2′-fluoro, 2′-O-methyl, 2′-O-allyl, or 2′-H. Other modifications to enhance stability or prolong biological activity can be internucleoside linkages, such as phosphorothioate, or base-modifications, such as locked nucleic acids (see, e.g., U.S. Pat. Nos. 6,670,461; 6,794,499; 6,268,490), or 5-methyluridine or 2′-O-methyl-5-methyluridine in place of uridine (see, e.g., U.S. Patent Application Publication No. 2006/0142230). Hence, dsRNA molecules of the instant disclosure can be modified to increase nuclease resistance or duplex stability while substantially retaining or having enhanced RNAi activity as compared to unmodified dsRNA.

In one embodiment, this disclosure features substituted or modified dsRNA molecules, such as phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, or alkylsilyl substitutions. For a review of oligonucleotide backbone modifications, see Hunziker and Leumann, Nucleic Acid Analogues: Synthesis and Properties, in Modern Synthetic Methods, VCH, 331-417, 1995; and Mesmaeker et al., ACS, 24-39, 1994.

In another embodiment, a conjugate molecule can be optionally attached to a dsRNA or analog thereof that decreases expression of a target gene by RNAi. For example, such conjugate molecules may be polyethylene glycol, human serum albumin, polyarginine, Gln-Asn polymer, or a ligand for a cellular receptor that can, for example, mediate cellular uptake (e.g., HIV TAT, see Vocero-Akbani et al., Nature Med. 5:23, 1999; see also U.S. Patent Application Publication No. 2004/0132161). Examples of specific conjugate molecules contemplated by the instant disclosure that can be attached to a dsRNA or analog thereof of this disclosure are described in Vargeese et al., U.S. Patent Application Publication No. 2003/0130186, and U.S. Patent Application Publication No. 2004/0110296. In another embodiment, a conjugate molecule is covalently attached to a dsRNA or analog thereof that decreases expression of a target gene by RNAi via a biodegradable linker. In certain embodiments, a conjugate molecule can be attached at the 3′-end of either the sense strand, the antisense strand, or both strands of a dsRNA molecule provided herein. In another embodiment, a conjugate molecule can be attached at the 5′-end of either the sense strand, the antisense strand, or both strands of the dsRNA or analog thereof. In yet another embodiment, a conjugate molecule is attached at both the 3′-end and 5′-end of either the sense strand, the antisense strand, or both strands of a dsRNA molecule, or any combination thereof. In further embodiments, a conjugate molecule of this disclosure comprises a molecule that facilitates delivery of a dsRNA or analog thereof into a biological system, such as a cell. A person of skill in the art can screen dsRNA of this disclosure having various conjugates to determine whether the dsRNA-conjugate possesses improved properties (e.g., pharmacokinetic profiles, bioavailability, stability) while maintaining the ability to mediate RNAi in, for example, an animal model as described herein or generally known in the art.

Methods for Selecting dsRNA Molecules Specific for a Target Gene

As indicated herein, the present disclosure also provides methods for selecting dsRNA and analogs thereof that are capable of specifically binding to a target gene (including a mRNA splice variant thereof) while being incapable of specifically binding or minimally binding to non-target genes (i.e, any nucleic acid sequence whose expression or activity is not to be altered). Thus, any of the target genes identified herein may be a non-target gene depending on what target gene(s) is identified for selecting dsRNAs capable of specifically binding to the target gene. The selection process disclosed herein is useful, for example, in eliminating dsRNAs analogs that are cytotoxic due to non-specific binding to, and subsequent degradation of, one or more non-target genes.

Methods of the present disclosure do not require a priori knowledge of the nucleotide sequence of every possible gene variant (including mRNA splice variants) targeted by the dsRNA or analog thereof. In one embodiment, the nucleotide sequence of the dsRNA is selected from a conserved region or consensus sequence of a target gene. In another embodiment, the nucleotide sequence of the dsRNA may be selectively or preferentially targeted to a certain sequence contained in an mRNA splice variant of a target gene.

In certain embodiments, methods are provided for selecting one or more dsRNA molecule that decreases expression of a target gene by RNAi, comprising a first strand that is complementary to a target mRNA and a second strand that is complementary to the first strand, wherein the first and second strands form a double-stranded region of about 15 to about 40 base pairs (see, e.g., target nucleotide sequences in the Sequence Listing identified herein), and wherein at least one uridine of the dsRNA molecule is replaced with a 5-methyluridine or 2-thioribothymidine or 2′-O-methyl-5-methyluridine, which methods employ “off-target” profiling whereby one or more dsRNA provided herein is contacted with a cell, either in vivo or in vitro, and total mRNA is collected for use in probing a microarray comprising oligonucleotides having one or more nucleotide sequence from a panel of known genes, including non-target genes (e.g., interferon). Within related embodiments, one or more dsRNA molecule that decreases expression of a target gene by RNAi may further comprise a third strand that is complementary to the first strand, wherein the first and third strands form a double-stranded region wherein the double-stranded region formed by the first and third strands is non-overlapping with a double-stranded region formed by the first and second strands. The “off-target” profile of the dsRNA provided herein is quantified by determining the number of non-target genes having reduced expression levels in the presence of the candidate dsRNAs. The existence of “off target” binding indicates a dsRNA is capable of specifically binding to one or more non-target gene messages. In certain embodiments, a dsRNA as provided herein (see, e.g., sequences in the Sequence Listing identified herein) applicable to therapeutic use will exhibit a greater stability, minimal interferon response, and little or no “off-target” binding.

Still further embodiments provide methods for selecting more efficacious dsRNA by using one or more reporter gene constructs comprising a constitutive promoter, such as a cytomegalovirus (CMV) or phosphoglycerate kinase (PGK) promoter, operably fused to, and capable of altering the expression of one or more reporter genes, such as a luciferase, chloramphenicol (CAT), or β-galactosidase, which, in turn, is operably fused in-frame with a dsRNA (such as one having a length between about 15 base-pairs and about 40 base-pairs or from about 5 nucleotides to about 24 nucleotides, or about 25 nucleotides to about 40 nucleotides) that contains a target sequence, as provided herein.

Individual reporter gene expression constructs may be co-transfected with one or more dsRNA or analog thereof. The capacity of a given dsRNA to reduce the expression level of target may be determined by comparing the measured reporter gene activity in cells transfected with or without a dsRNA molecule of interest.

Certain embodiments disclosed herein provide methods for selecting one or more modified dsRNA molecule(s) that employ the step of predicting the stability of a dsRNA duplex. In some embodiments, such a prediction is achieved by employing a theoretical melting curve wherein a higher theoretical melting curve indicates an increase in dsRNA duplex stability and a concomitant decrease in cytotoxic effects. Alternatively, stability of a dsRNA duplex may be determined empirically by measuring the hybridization of a single RNA analog strand as described herein to a complementary target gene within, for example, a polynucleotide array. The melting temperature (i.e., the Tₘ value) for each modified RNA and complementary RNA immobilized on the array can be determined and, from this Tₘ value, the relative stability of the modified RNA pairing with a complementary RNA molecule determined.

For example, Kawase et al. (Nucleic Acids Res. 14:7727, 1986) have described an analysis of the nucleotide-pairing properties of Di (inosine) to A, C, G, and T, which was achieved by measuring the hybridization of oligonucleotides (ODNs) with Di in various positions to complementary sets of ODNs made as an array. The relative strength of nucleotide-pairing is I-C>I-A>I-G≈I-T. Generally, Di containing duplexes showed lower Tₘ values when compared to the corresponding wild type (WT) nucleotide pair. The stabilization of Di by pairing was in order of Dc>Da>Dg>Dt>Du. As a person of skill in the art would understand, although universal-binding nucleotides are used herein as an example of determining duplex stability (i.e., the Tₘ value), other nucleotide substitutions (e.g., 5-methyluridine for uridine) or further modifications (e.g., a ribose modification at the 2′-position) can also be evaluated by these or similar methods.

In still further embodiments of the presently disclosed methods, one or more anti-codon within an antisense strand of a dsRNA molecule or analog thereof is substituted with a universal-binding nucleotide in a second or third position in the anti-codon of the antisense strand. By substituting a universal-binding nucleotide for a first or second position, the one or more first or second position nucleotide-pair substitution allows the substituted dsRNA molecule to specifically bind to mRNA wherein a first or a second position nucleotide-pair substitution has occurred, wherein the one or more nucleotide-pair substitution results in an amino acid change in the corresponding gene product.

Any of these methods of identifying dsRNA of interest can also be used to examine a dsRNA that decreases expression of a target gene by RNA interference, comprising a first strand that is complementary to a target T mRNA and a second and third strand that have non-overlapping complementarity to the first strand, wherein the first and at least one of the second or third strand form a double-stranded region of about 5 to about 13 base pairs; wherein at least one pyrimidine of the dsRNA comprises a pyrimidine nucleoside according to Formula I or II:

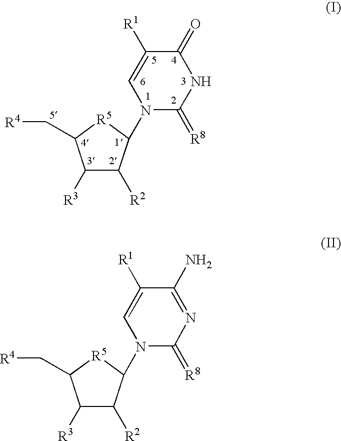

wherein

R¹ and R² are each independently a —H, —OH, —OCH₃, —OCH₂OCH₂CH₃, —OCH₂CH₂OCH₃, halogen, substituted or unsubstituted C₁-C₁₀ alkyl, alkoxy, alkoxyalkyl, hydroxyalkyl, carboxyalkyl, alkylsulfonylamino, aminoalkyl, dialkylamino, alkylaminoalkyl, dialkylaminoalkyl, haloalkyl, trifluoromethyl, cycloalkyl, (cycloalkyl)alkyl, substituted or unsubstituted C₂-C₁₀ alkenyl, substituted or unsubstituted —O-allyl, —O—CH₂CH═CH₂, —O—CH═CHCH₃, substituted or unsubstituted C₂-C₁₀ alkynyl, carbamoyl, carbamyl, carboxy, carbonylamino, substituted or unsubstituted aryl, substituted or unsubstituted aralkyl, —NH₂, —NO₂, —C≡N, or heterocyclo group;

R³ and R⁴ are each independently a hydroxyl, a protected hydroxyl, or an internucleoside linking group; and

R⁵ and R⁸ are independently O or S.

In certain embodiments, at least one nucleoside is according to Formula I in which R¹ is methyl and R² is —OH, or R¹ is methyl, R² is —OH, and R⁸ is S. In certain embodiments, at least one nucleoside is according to Formula I in which R¹ is methyl and R² is —O-methyl, or R¹ is methyl, R² is —O-methyl, and R⁸ is O. In other embodiments, the internucleoside linking group covalently links from about 5 to about 40 nucleosides.

### Compositions and Methods of Use

As set forth herein, dsRNA of the instant disclosure are designed to target a target gene (including one or more mRNA splice variant thereof) that is expressed at an elevated level or continues to be expressed when it should not, and is a causal or contributing factor associated with, for example, atherosclerosis, diabetes mellitus, and cerebrovascular disease, state, or adverse condition. In this context, a dsRNA or analog thereof of this disclosure will effectively downregulate expression of a target gene to levels that prevent, alleviate, or reduce the severity or recurrence of one or more associated disease symptoms. Alternatively, for various distinct disease models in which expression of a target gene is not necessarily elevated as a consequence or sequel of disease or other adverse condition, down regulation of a target gene will nonetheless result in a therapeutic result by lowering gene expression (i.e., to reduce levels of a selected mRNA or protein product of a target gene). Furthermore, dsRNAs of this disclosure may be targeted to lower expression of target, which can result in upregulation of a “downstream” gene whose expression is negatively regulated, directly or indirectly, by a target protein. The dsRNA molecules of the instant disclosure comprise useful reagents and can be used in methods for a variety of therapeutic, diagnostic, target validation, genomic discovery, genetic engineering, and pharmacogenomic applications.

In certain embodiments, aqueous suspensions contain dsRNA of this disclosure in admixture with suitable excipients, such as suspending agents or dispersing or wetting agents. Exemplary suspending agents include sodium carboxymethylcellulose, methylcellulose, hydropropyl-methylcellulose, sodium alginate, polyvinylpyrrolidone, gum tragacanth and gum acacia. Representative dispersing or wetting agents include naturally-occurring phosphatides (e.g., lecithin), condensation products of an alkylene oxide with fatty acids (e.g., polyoxyethylene stearate), condensation products of ethylene oxide with long chain aliphatic alcohols (e.g., heptadecaethyleneoxycetanol), condensation products of ethylene oxide with partial esters derived from fatty acids and hexitol (e.g., polyoxyethylene sorbitol monooleate), or condensation products of ethylene oxide with partial esters derived from fatty acids and hexitol anhydrides (e.g., polyethylene sorbitan monooleate). In certain embodiments, the aqueous suspensions can optionally contain one or more preservatives (e.g., ethyl or n-propyl-p-hydroxybenzoate), one or more coloring agents, one or more flavoring agents, or one or more sweetening agents (e.g., sucrose, saccharin). In additional embodiments, dispersible powders and granules suitable for preparation of an aqueous suspension by the addition of water provide dsRNA of this disclosure in admixture with a dispersing or wetting agent, suspending agent and optionally one or more preservative, coloring agent, flavoring agent, or sweetening agent.

The present disclosure includes dsRNA compositions prepared for storage or administration that include a pharmaceutically effective amount of a desired compound in a pharmaceutically acceptable carrier or diluent. Acceptable carriers or diluents for therapeutic use are well known in the pharmaceutical art, and are described, for example, in Remington's Pharmaceutical Sciences, Mack Publishing Co., A. R. Gennaro edit., 1985, hereby incorporated by reference herein. In certain embodiments, pharmaceutical compositions of this disclosure can optionally include preservatives, antioxidants, stabilizers, dyes, flavoring agents, or any combination thereof. Exemplary preservatives include sodium benzoate, sorbic acid, chlorobutanol, and esters of p-hydroxybenzoic acid.

The dsRNA compositions of the instant disclosure can be effectively employed as pharmaceutically-acceptable formulations. Pharmaceutically-acceptable formulations prevent, alter the occurrence or severity of, or treat (alleviate one or more symptom(s) to a detectable or measurable extent) of a disease state or other adverse condition in a subject. A pharmaceutically acceptable formulation includes salts of the above compounds, e.g., acid addition salts, such as salts of hydrochloric acid, hydrobromic acid, acetic acid, or benzene sulfonic acid. A pharmaceutical composition or formulation refers to a composition or formulation in a form suitable for administration into a cell, or a subject such as a human (e.g., systemic administration). The formulations of the present disclosure, having an amount of dsRNA sufficient to treat or prevent a disorder associated with target gene expression are, for example, suitable for topical (e.g., creams, ointments, skin patches, eye drops, ear drops) application or administration. Other routes of administration include oral, parenteral, sublingual, bladder wash-out, vaginal, rectal, enteric, suppository, nasal, and inhalation. The term parenteral, as used herein, includes subcutaneous, intravenous, intramuscular, intraarterial, intraabdominal, intraperitoneal, intraarticular, intraocular or retrobulbar, intraaural, intrathecal, intracavitary, intracelial, intraspinal, intrapulmonary or transpulmonary, intrasynovial, and intraurethral injection or infusion techniques. The pharmaceutical compositions of the present disclosure are formulated to allow the dsRNA contained therein to be bioavailable upon administration to a subject.

In further embodiments, dsRNA of this disclosure can be formulated as oily suspensions or emulsions (e.g., oil-in-water) by suspending dsRNA in, for example, a vegetable oil (e.g., arachis oil, olive oil, sesame oil or coconut oil) or a mineral oil (e.g., liquid paraffin). Suitable emulsifying agents can be naturally-occurring gums (e.g., gum acacia or gum tragacanth), naturally-occurring phosphatides (e.g., soy bean, lecithin, esters or partial esters derived from fatty acids and hexitol), anhydrides (e.g., sorbitan monooleate), or condensation products of partial esters with ethylene oxide (e.g., polyoxyethylene sorbitan monooleate). In certain embodiments, the oily suspensions or emulsions can optionally contain a thickening agent, such as beeswax, hard paraffin or acetyl alcohol. In related embodiments, sweetening agents and flavoring agents can optionally be added to provide palatable oral preparations. In yet other embodiments, these compositions can be preserved by optionally adding an anti-oxidant, such as ascorbic acid.

In further embodiments, dsRNA of this disclosure can be formulated as syrups and elixirs with sweetening agents (e.g., glycerol, propylene glycol, sorbitol, glucose or sucrose). Such formulations can also contain a demulcent, preservative, flavoring, coloring agent, or any combination thereof. In other embodiments, pharmaceutical compositions comprising dsRNA of this disclosure can be in the form of a sterile, injectable aqueous or oleaginous suspension. The sterile injectable preparation can also be a sterile, injectable solution or suspension in a non-toxic parenterally acceptable diluent or solvent (e.g., as a solution in 1,3-butanediol). Among the exemplary acceptable vehicles and solvents useful in the compositions of this disclosure is water, Ringer's solution, or isotonic sodium chloride solution. In addition, sterile, fixed oils may be employed as a solvent or suspending medium for the dsRNA of this disclosure. For this purpose, any bland fixed oil can be employed including synthetic mono- or diglycerides. In addition, fatty acids such as oleic acid find use in the preparation of parenteral formulations.

Within certain embodiments of this disclosure, pharmaceutical compositions and methods are provided that feature the presence or administration of one or more dsRNA or analogs thereof of this disclosure, combined, complexed, or conjugated with a polypeptide, optionally formulated with a pharmaceutically-acceptable carrier, such as a diluent, stabilizer, buffer, or the like. The negatively charged dsRNA molecules of this disclosure may be administered to a patient by any standard means, with or without stabilizers, buffers, or the like, to form a composition suitable for treatment. When it is desired to use a liposome delivery mechanism, standard protocols for formation of liposomes can be followed. The compositions of the present disclosure may also be formulated and used as a tablet, capsule or elixir for oral administration, suppository for rectal administration, sterile solution, or suspension for injectable administration, either with or without other compounds known in the art. Thus, dsRNAs of the present disclosure may be administered in any form, such as nasally, transdermally, parenterally, or by local injection.

In accordance with this disclosure, dsRNA molecules (optionally substituted or modified or conjugated), compositions thereof, and methods for inhibiting expression of a target gene in a cell or organism are provided. In certain embodiments, this disclosure provides methods and dsRNA compositions for treating a subject, including a human cell, tissue or individual, having a disease or at risk of developing a disease caused by or associated with the expression of a target gene. In one embodiment, the method includes administering a dsRNA of this disclosure or a pharmaceutical composition containing the dsRNA to a cell or an organism, such as a mammal, such that expression of the target gene is silenced. Subjects (e.g., mammalian, human) amendable for treatment using the dsRNA molecules (optionally substituted or modified or conjugated), compositions thereof, and methods of the present disclosure include those suffering from one or more disease or condition mediated, at least in part, by overexpression or inappropriate expression of a target gene, or which are amenable to treatment by reducing expression of a target protein, including coronary artery disease (i.e., coronary heart disease, ischaemic heart disease), atherosclerosis, diabetes mellitus, dyslipidemia (e.g., hyperlipidemia), peripheral vascular and ischemic cerebrovascular disease, and risk of ischemic stroke (cerebral thrombosis and cerebral embolisms) and hemorrhagic stroke (cerebral hemorrhage and subarachnoid hemorrhage), cancer (e.g., lung cancer, heptacellular carcinoma, bladder cancer, and pancreatic cancer). Within exemplary embodiments, the compositions and methods of this disclosure are also useful as therapeutic tools to regulate expression of target to treat or prevent symptoms of, for example, the conditions listed herein.

In any of the methods disclosed herein there may be used with one or more dsRNA, or substituted or modified dsRNA, as described herein, comprising a first strand that is complementary to a human target mRNA and a second strand and a third strand that is each complementary to non-overlapping regions of the first strand, wherein the second strand and third strands can anneal with the first strand to form at least two double-stranded regions spaced apart by up to 10 nucleotides and thereby forming a gap between the second and third strands, and wherein the mdRNA molecule optionally includes at least one double-stranded region of 5 base pairs to 13 base pairs. In other embodiments, subjects can be effectively treated, prophylactically or therapeutically, by administering an effective amount of one or more dsRNA having a first strand that is complementary to a human target mRNA and a second strand and a third strand that is each complementary to non-overlapping regions of the first strand, wherein the second strand and third strands can anneal with the first strand to form at least two double-stranded regions spaced apart by up to 10 nucleotides and thereby forming a gap between the second and third strands, and wherein the mdRNA molecule optionally includes at least one double-stranded region of 5 base pairs to 13 base pairs and at least one pyrimidine of the mdRNA is substituted with a pyrimidine nucleoside according to Formula I or II:

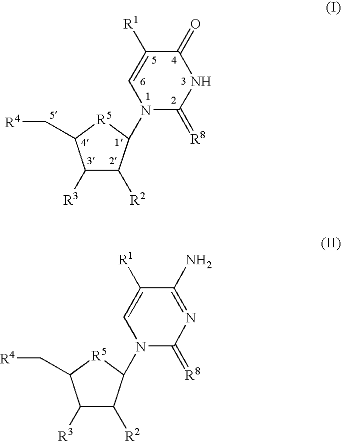

wherein

R¹ and R² are each independently a —H, —OH, —OCH₃, —OCH₂OCH₂CH₃, —OCH₂CH₂OCH₃, halogen, substituted or unsubstituted C₁-C₁₀ alkyl, alkoxy, alkoxyalkyl, hydroxyalkyl, carboxyalkyl, alkylsulfonylamino, aminoalkyl, dialkylamino, alkylaminoalkyl, dialkylaminoalkyl, haloalkyl, trifluoromethyl, cycloalkyl, (cycloalkyl)alkyl, substituted or unsubstituted C₂-C₁₀ alkenyl, substituted or unsubstituted —O-allyl, —O—CH₂CH═CH₂, —O—CH═CHCH₃, substituted or unsubstituted C₂-C₁₀ alkynyl, carbamoyl, carbamyl, carboxy, carbonylamino, substituted or unsubstituted aryl, substituted or unsubstituted aralkyl, —NH₂, —NO₂, —C≡N, or heterocyclo group;

R³ and R⁴ are each independently a hydroxyl, a protected hydroxyl, or an internucleoside linking group; and

R⁵ and R⁸ are independently O or S.

In certain embodiments, at least one nucleoside is according to Formula I in which R¹ is methyl and R² is —OH, or R¹ is methyl, R² is —OH, and R⁸ is S. In other embodiments, the internucleoside linking group covalently links from about 5 to about 40 nucleosides.

In any of the methods described herein, the dsRNA used may include multiple modifications. For example, a dsRNA can have at least one 5-methyluridine, 2′-O-methyl-5-methyluridine, LNA, 2′-methoxy, 2′-fluoro, 2′-deoxy, phosphorothioate linkage, inverted base terminal cap, or any combination thereof. In certain exemplary methods, a dsRNA will have from one to all 5-methyluridines and have up to about 75% LNA.

In other exemplary methods, a dsRNA will have from one to all 5-methyluridines and have up to about 75% 2′-methoxy provided the 2′-methoxy are not at the Argonaute cleavage site. In still other exemplary methods, a dsRNA will have from one to all 5-methyluridines and have up to about 100% 2′-fluoro substitutions. In further exemplary methods, a dsRNA will have from one to all 5-methyluridines and have up to about 75% 2′-deoxy.

In further exemplary methods, a dsRNA will have up to about 75% LNA and have up to about 75% 2′-methoxy. In still other embodiments, a dsRNA will have up to about 75% LNA and have up to about 100% 2′-fluoro. In further exemplary methods, a dsRNA will have up to about 75% LNA and have up to about 75% 2′-deoxy. In further exemplary methods, a dsRNA will have up to about 75% 2′-methoxy and have up to about 100% 2′-fluoro. In further exemplary methods, a dsRNA will have up to about 75% 2′-methoxy and have up to about 75% 2′-deoxy. In further embodiments, a dsRNA will have up to about 100% 2′-fluoro and have up to about 75% 2′-deoxy.

In other exemplary methods for using multiply modified dsRNA, a dsRNA will have from one to all uridines substituted with 5-methyluridine, up to about 75% LNA, and up to about 75% 2′-methoxy. In still further exemplary methods, a dsRNA will have from one to all 5-methyluridines, up to about 75% LNA, and up to about 100% 2′-fluoro. In further exemplary methods, a dsRNA will have from one to all 5-methyluridines, up to about 75% LNA, and up to about 75% 2′-deoxy. In further exemplary methods, a dsRNA will have from one to all 5-methyluridines, up to about 75% 2′-methoxy, and up to about 75% 2′-fluoro. In further exemplary methods, a dsRNA will have from one to all 5-methyluridines, up to about 75% 2′-methoxy, and up to about 75% 2′-deoxy. In more exemplary methods, a dsRNA will have from one to all 5-methyluridines, up to about 100% 2′-fluoro, and up to about 75% 2′-deoxy. In yet other exemplary methods, a dsRNA will have from one to all 5-methyluridines, up to about 75% LNA, up to about 75% 2′-methoxy, up to about 100% 2′-fluoro, and up to about 75% 2′-deoxy. In other exemplary methods, a dsRNA will have up to about 75% LNA, up to about 75% 2′-methoxy, and up to about 100% 2′-fluoro. In further exemplary methods, a dsRNA will have up to about 75% LNA, up to about 75% 2′-methoxy, and up to about 75% 2′-deoxy. In more exemplary methods, a dsRNA will have up to about 75% LNA, up to about 100% 2′-fluoro, and up to about 75% 2′-deoxy. In still further exemplary methods, a dsRNA will have up to about 75% 2′-methoxy, up to about 100% 2′-fluoro, and up to about 75% 2′-deoxy.

In any of these exemplary methods for using multiply modified dsRNA, the dsRNA may further comprise up to 100% phosphorothioate internucleoside linkages, from one to ten or more inverted base terminal caps, or any combination thereof. Additionally, any of these dsRNA may have these multiple modifications on one strand, two strands, three strands, a plurality of strands, or all strands, or on the same or different nucleoside within a dsRNA molecule. Finally, in any of these multiple modification dsRNA, the dsRNA must have gene silencing activity.

In further embodiments, subjects can be effectively treated, prophylactically or therapeutically, by administering an effective amount of one or more dsRNA, or substituted or modified dsRNA as described herein, having a first strand that is complementary to a target mRNA and a second strand and a third strand that is each complementary to non-overlapping regions of the first strand, wherein the second strand and third strands can anneal with the first strand to form at least two double-stranded regions spaced apart by up to 10 nucleotides and thereby forming a gap between the second and third strands, and wherein the combined double-stranded regions total about 15 base pairs to about 40 base pairs and the mdRNA molecule optionally has blunt ends. In still further embodiments, methods disclosed herein there may be used with one or more dsRNA that comprises a first strand that is complementary to a target mRNA and a second strand and a third strand that is each complementary to non-overlapping regions of the first strand, wherein the second strand and third strands can anneal with the first strand to form at least two double-stranded regions spaced apart by up to 10 nucleotides and thereby forming a gap between the second and third strands, and wherein the mdRNA molecule optionally includes at least one double-stranded region of 5 base pairs to 13 base pairs, the mdRNA molecule optionally has blunt ends, and at least one pyrimidine of the mdRNA is substituted with a pyrimidine nucleoside according to Formula I or II:

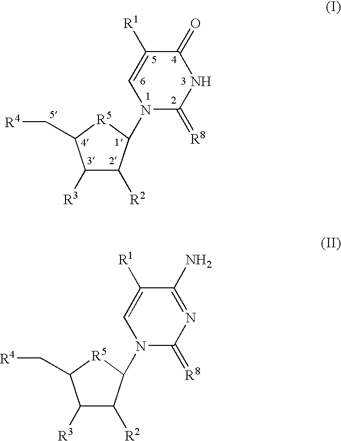

wherein

R¹ and R² are each independently a —H, —OH, —OCH₃, —OCH₂OCH₂CH₃, —OCH₂CH₂OCH₃, halogen, substituted or unsubstituted C₁-C₁₀ alkyl, alkoxy, alkoxyalkyl, hydroxyalkyl, carboxyalkyl, alkylsulfonylamino, aminoalkyl, dialkylamino, alkylaminoalkyl, dialkylaminoalkyl, haloalkyl, trifluoromethyl, cycloalkyl, (cycloalkyl)alkyl, substituted or unsubstituted C₂-C₁₀ alkenyl, substituted or unsubstituted —O-allyl, —O—CH₂CH═CH₂, —O—CH═CHCH₃, substituted or unsubstituted C₂-C₁₀ alkynyl, carbamoyl, carbamyl, carboxy, carbonylamino, substituted or unsubstituted aryl, substituted or unsubstituted aralkyl, —NH₂, —NO₂, —C≡N, or heterocyclo group;

R³ and R⁴ are each independently a hydroxyl, a protected hydroxyl, or an internucleoside linking group; and

R⁵ and R⁸ are independently O or S.

In certain embodiments, at least one nucleoside is according to Formula I in which R¹ is methyl and R² is —OH, or R¹ is methyl, R² is —OH, and R⁸ is S.

In certain embodiments, at least one nucleoside is according to Formula I in which R¹ is methyl and R² is —O-methyl, or R¹ is methyl, R² is —O-methyl, and R⁸ is O. In other embodiments, the internucleoside linking group covalently links from about 5 to about 40 nucleosides.

Within additional aspects of this disclosure, combination formulations and methods are provided comprising an effective amount of one or more dsRNA of the present disclosure in combination with one or more secondary or adjunctive active agents that are formulated together or administered coordinately with the dsRNA of this disclosure to control a target gene-associated disease or condition. Useful adjunctive therapeutic agents in these combinatorial formulations and coordinate treatment methods include, for example, dsRNAs that target and decrease the expression of other genes whose abbarent expression is related to a disease or condition described herein (e.g., bladder cancer and/liver cancer), enzymatic nucleic acid molecules, allosteric nucleic acid molecules, antisense, decoy, or aptamer nucleic acid molecules, antibodies such as monoclonal antibodies, small molecules and other organic or inorganic compounds including metals, salts and ions, and other drugs and active agents indicated for treating a target gene-associated disease or condition, including chemotherapeutic agents used to treat cancer, steroids, non-steroidal anti-inflammatory drugs (NSAIDs), tyrosine kinase inhibitors, or the like.

Exemplary chemotherapeutic agents include alkylating agents (e.g., cisplatin, oxaliplatin, carboplatin, busulfan, nitrosoureas, nitrogen mustards, uramustine, temozolomide), antimetabolites (e.g., aminopterin, methotrexate, mercaptopurine, fluorouracil, cytarabine), taxanes (e.g., paclitaxel, docetaxel), anthracyclines (e.g., doxorubicin, daunorubicin, epirubicin, idaruicin, mitoxantrone, valrubicin), bleomycin, mytomycin, actinomycin, hydroxyurea, topoisomerase inhibitors (e.g., camptothecin, topotecan, irinotecan, etoposide, teniposide), monoclonal antibodies (e.g., alemtuzumab, bevacizumab, cetuximab, gemtuzumab, panitumumab, rituximab, tositumomab, trastuzumab), vinca alkaloids (e.g., vincristine, vinblastine, vindesine, vinorelbine), cyclophosphamide, prednisone, leucovorin, oxaliplatin.

Some adjunctive therapies may be directed at targets that interact or associate with the target gene or affect specific target gene biological activities. Adjunctive therapies include statins (e.g., rosuvastatin, lovastatin, atorvastatin, cerivastatin, fluvastatin, mevastatin, pitavastatin, pravastatin, simvastatin), bile acid-binding resins, stanol and sterol esters from plants, and inhibitors of cholesterol absorption, fibrates (e.g., fenofibrate, bezafibrate, ciprofibrate, clofibrate, gemfibrozil), niacin, fish-oils, ezetimibe, amlodipine, other lipid-altering agents, additional small molecules, rationally designed peptides, and antibodies or fragments thereof.

Genes may be targeted via the RNAi pathway by way of a dsRNA and used in combination with two or more dsRNAs of this disclosure

To practice the coordinate administration methods of this disclosure, a dsRNA is administered, simultaneously or sequentially, in a coordinated treatment protocol with one or more of the secondary or adjunctive therapeutic agents contemplated herein. The coordinate administration may be done in any order, and there may be a time period while only one or both (or all) active therapeutic agents, individually or collectively, exert their biological activities. A distinguishing aspect of all such coordinate treatment methods is that the dsRNA present in a composition elicits some favorable clinical response, which may or may not be in conjunction with a secondary clinical response provided by the secondary therapeutic agent. For example, the coordinate administration of the dsRNA with a secondary therapeutic agent as contemplated herein can yield an enhanced (synergistic) therapeutic response beyond the therapeutic response elicited by either or both the purified dsRNA or secondary therapeutic agent alone.

In another embodiment, a dsRNA of this disclosure can include a conjugate member on one or more of the terminal nucleotides of a dsRNA. The conjugate member can be, for example, a lipophile, a terpene, a protein binding agent, a vitamin, a carbohydrate, or a peptide. For example, the conjugate member can be naproxen, nitroindole (or another conjugate that contributes to stacking interactions), folate, ibuprofen, or a C5 pyrimidine linker. In other embodiments, the conjugate member is a glyceride lipid conjugate (e.g., a dialkyl glyceride derivatives), vitamin E conjugates, or thio-cholesterols. Additional conjugate members include peptides that function, when conjugated to a modified dsRNA of this disclosure, to facilitate delivery of the dsRNA into a target cell, or otherwise enhance delivery, stability, or activity of the dsRNA when contacted with a biological sample (e.g., a target cell expressing the target gene). Exemplary peptide conjugate members for use within these aspects of this disclosure, include peptides PN27, PN28, PN29, PN58, PN61, PN73, PN158, PN159, PN173, PN182, PN183, PN202, PN204, PN250, PN361, PN365, PN404, PN453, PN509, and PN963, described, for example, in U.S. Patent Application Publication Nos. 2006/0040882 and 2006/0014289, and U.S. Provisional Patent Application Nos. 60/822,896 and 60/939,578; and PCT Application PCT/US2007/075744, which are all incorporated herein by reference. In certain embodiments, when peptide conjugate partners are used to enhance delivery of dsRNA of this disclosure, the resulting dsRNA formulations and methods will often exhibit further reduction of an interferon response in target cells as compared to dsRNAs delivered in combination with alternate delivery vehicles, such as lipid delivery vehicles (e.g., Lipofectamine™).

In still another embodiment, a dsRNA or analog thereof of this disclosure may be conjugated to the polypeptide and admixed with one or more non-cationic lipids or a combination of a non-cationic lipid and a cationic lipid to form a composition that enhances intracellular delivery of the dsRNA as compared to delivery resulting from contacting the target cells with a naked dsRNA. In more detailed aspects of this disclosure, the mixture, complex or conjugate comprising a dsRNA and a polypeptide can be optionally combined with (e.g., admixed or complexed with) a cationic lipid, such as Lipofectine™. To produce these compositions comprised of a polypeptide, dsRNA and a cationic lipid, the dsRNA and peptide may be mixed together first in a suitable medium such as a cell culture medium, after which the cationic lipid is added to the mixture to form a dsRNA/delivery peptide/cationic lipid composition. Optionally, the peptide and cationic lipid can be mixed together first in a suitable medium such as a cell culture medium, followed by the addition of the dsRNA to form the dsRNA/delivery peptide/cationic lipid composition.

This disclosure also features the use of dsRNA compositions comprising surface-modified liposomes containing, for example, poly(ethylene glycol) lipids (PEG-modified, or long-circulating liposomes or stealth liposomes) (Lasic et al., Chem. Rev. 95:2601, 1995; Ishiwata et al., Chem. Pharm. Bull. 43:1005, 1995; Lasic et al., Science 267:1275, 1995; Oku et al., Biochim. Biophys. Acta 1238:86, 1995; Liu et al., J. Biol. Chem. 42:24864, 1995; PCT Publication Nos. WO 96/10391; WO 96/10390; WO 96/10392).

In another embodiment, compositions are provided for targeting dsRNA molecules of this disclosure to specific cell types, such as hepatocytes. For example, dsRNA can be complexed or conjugated glycoproteins or synthetic glycoconjugates glycoproteins or synthetic glycoconjugates having branched galactose (e.g., asialoorosomucoid), N-acetyl-D-galactosamine, or mannose (see, e.g., Wu and Wu, J. Biol. Chem. 262:4429, 1987; Baenziger and Fiete, Cell 22: 611, 1980; Connolly et al., J. Biol. Chem. 257:939, 1982; Lee and Lee, Glycoconjugate J. 4:317, 1987; Ponpipom et al., J. Med. Chem. 24:1388, 1981) for a targeted delivery to, for example, the liver.

A pharmaceutically effective dose is that dose required to prevent, inhibit the occurrence of, or treat (alleviate a symptom to some extent, preferably all of the symptoms) a disease state. The pharmaceutically effective dose depends on the type of disease, the composition used, the route of administration, the type of subject being treated, the physical characteristics of the specific subject under consideration for treatment, concurrent medication, and other factors that those skilled in the medical arts will recognize. For example, an amount between 0.1 mg/kg and 100 mg/kg body weight/day of active ingredients may be administered depending on the potency of a dsRNA of this disclosure.

A specific dose level for any particular patient depends upon a variety of factors including the activity of the specific compound employed, age, body weight, general health, sex, diet, time of administration, route of administration, rate of excretion, drug combination, and the severity of the particular disease undergoing therapy. Following administration of dsRNA compositions as disclosed herein, test subjects will exhibit about a 10% up to about a 99% reduction in one or more symptoms associated with the disease or disorder being treated, as compared to placebo-treated or other suitable control subjects.

Dosage levels in the order of about 0.1 mg to about 140 mg per kilogram of body weight per day can be useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

A dosage form of a dsRNA or composition thereof of this disclosure can be liquid, an emulsion, or a micelle, or in the form of an aerosol or droplets. A dosage form of a dsRNA or composition thereof of this disclosure can be solid, which can be reconstituted in a liquid prior to administration. The solid can be administered as a powder. The solid can be in the form of a capsule, tablet, or gel. In addition to in vivo gene inhibition, a skilled artisan will appreciate that the dsRNA and analogs thereof of the present disclosure are useful in a wide variety of in vitro applications, such as scientific and commercial research (e.g., elucidation of physiological pathways, drug discovery and development), and medical and veterinary diagnostics.

Nucleic acid molecules and polypeptides can be administered to cells by a variety of methods known to those of skill in the art, including administration within formulations that comprise a dsRNA alone, a dsRNA and a polypeptide complex/conjugate alone, or that further comprise one or more additional components, such as a pharmaceutically acceptable carrier, diluent, excipient, adjuvant, emulsifier, stabilizer, preservative, or the like. Other exemplary substances used to approximate physiological conditions include pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments, the dsRNA and compositions thereof can be encapsulated in liposomes, administered by iontophoresis, or incorporated into other vehicles, such as hydrogels, cyclodextrins, biodegradable nanocapsules, bioadhesive microspheres, or proteinaceous vectors (see, e.g., PCT Publication No. WO 00/53722). In certain embodiments of this disclosure, the dsRNA may be administered in a time release formulation, for example, in a composition that includes a slow release polymer. The dsRNA can be prepared with carriers that will protect against rapid release, for example, a controlled release vehicle such as a polymer, microencapsulated delivery system, or bioadhesive gel. Prolonged delivery of the dsRNA, in various compositions of this disclosure can be brought about by including in the composition agents that delay absorption, for example, aluminum monosterate hydrogels and gelatin.

Alternatively, a dsRNA composition of this disclosure can be locally delivered by direct injection or by use of, for example, an infusion pump. Direct injection of dsRNAs of this disclosure, whether subcutaneous, intramuscular, or intradermal, can be done by using standard needle and syringe methodologies or by needle-free technologies, such as those described in Conry et al., Clin. Cancer Res. 5:2330, 1999 and PCT Publication No. WO 99/31262.

The dsRNA of this disclosure and compositions thereof may be administered to subjects by a variety of mucosal administration modes, including oral, rectal, vaginal, intranasal, intrapulmonary, or transdermal delivery, or by topical delivery to the eyes, ears, skin, or other mucosal surfaces. In one embodiment, the mucosal tissue layer includes an epithelial cell layer, which can be pulmonary, tracheal, bronchial, alveolar, nasal, buccal, epidermal, or gastrointestinal. Compositions of this disclosure can be administered using conventional actuators, such as mechanical spray devices, as well as pressurized, electrically activated, or other types of actuators. The dsRNAs can also be administered in the form of suppositories, e.g., for rectal administration. For example, these compositions can be mixed with an excipient that is solid at room temperature but liquid at the rectal temperature so that the dsRNA is released. Such materials include, for example, cocoa butter and polyethylene glycols.

Further methods for delivery of nucleic acid molecules, such as the dsRNAs of this disclosure, are described, for example, in Boado et al., J. Pharm. Sci. 87:1308, 1998; Tyler et al., FEBS Lett. 421:280, 1999; Pardridge et al., Proc. Nat'l Acad. Sci. USA 92:5592, 1995; Boado, Adv. Drug Delivery Rev. 15:73, 1995; Aldrian-Herrada et al., Nucleic Acids Res. 26:4910, 1998; Tyler et al., Proc. Nat'l Acad. Sci. USA 96:7053-7058, 1999; Akhtar et al., Trends Cell Bio. 2:139, 1992; “Delivery Strategies for Antisense Oligonucleotide Therapeutics,” ed. Akhtar, 1995, Maurer et al., Mol. Membr. Biol. 16:129, 1999; Hofland and Huang, Handb. Exp. Pharmacol 137:165, 1999; and Lee et al., ACS Symp. Ser. 752:184, 2000; PCT Publication No. WO 94/02595.

All U.S. patents, U.S. patent application publications, U.S. patent applications, foreign patents, foreign patent applications, non-patent publications, figures, tables, and websites referred to in this specification are expressly incorporated herein by reference, in their entirety.

### EXAMPLES

### Example 1

### Knockdown of Gene Expression by mdRNA

The gene silencing activity of dsRNA as compared to nicked or gapped versions of the same dsRNA was examined using a dual fluorescence assay. A total of 22 different genes were targeted at ten different sites each (see Table 1).

A Dicer substrate dsRNA molecule was used, which has a 25 nucleotide sense strand, a 27 nucleotide antisense strand, and a two deoxynucleotide overhang at the 3′-end of the antisense strand (referred to as a 25/27 dsRNA). The nicked version of each dsRNA Dicer substrate has a nick at one of positions 9 to 16 on the sense strand as measured from the 5′-end of the sense strand. For example, an ndsRNA having a nick at position 11 has three strands—a 5′-sense strand of 11 nucleotides, a 3′-sense strand of 14 nucleotides, and an antisense strand of 27 nucleotides (which is also referred to as an N11-14/27 mdRNA). In addition, each of the sense strands of the ndsRNA have three locked nucleic acids (LNAs) evenly distributed along each sense fragment. If the nick is at position 9, then the LNAs can be found at positions 2, 6, and 9 of the 5′ sense strand fragment and at positions 11, 18, and 23 of the 3′ sense strand fragment. If the nick is at position 10, then the LNAs can be found at positions 2, 6, and 10 of the 5′ sense strand fragment and at positions 12, 18, and 23 of the 3′ sense strand fragment. If the nick is at position 11, then the LNAs can be found at positions 2, 6, and 11 of the 5′ sense strand fragment and at positions 13, 18, and 23 of the 3′ sense strand fragment. If the nick is at position 12, then the LNAs can be found at positions 2, 6, and 12 of the 5′ sense strand fragment and at positions 14, 18, and 23 of the 3′ sense strand fragment. If the nick is at position 13, then the LNAs can be found at positions 2, 7, and 13 of the 5′ sense strand fragment and at positions 15, 18, and 23 of the 3′ sense strand fragment. If the nick is at position 14, then the LNAs can be found at positions 2, 7, and 14 of the 5′ sense strand fragment and at positions 16, 18, and 23 of the 3′ sense strand fragment. If the nick is at position 15, then the LNAs can be found at positions 2, 8, and 15 of the 5′ sense strand fragment and at positions 17, 19, and 23 of the 3′ sense strand fragment. If the nick is at position 16, then the LNAs can be found at positions 2, 8, and 16 of the 5′ sense strand fragment and at positions 18, 19, and 23 of the 3′ sense strand fragment. Similarly, a gapped version of each dsRNA Dicer substrate has a single nucleotide missing at one of positions 10 to 17 on the sense strand as measured from the 5′-end of the sense strand. For example, a gdsRNA having a gap at position 11 has three strands—a 5′-sense strand of 11 nucleotides, a 3′-sense strand of 13 nucleotides, and an antisense strand of 27 nucleotides (which is also referred to as G11-(1)-13/27 mdRNA). In addition, each of the sense strands of the gdsRNA contain three locked nucleic acids (LNAs) evenly distributed along each sense fragment (as described for the nicked counterparts).

In sum, three dsRNA were tested at each of the ten different sites per gene—an unmodified dsRNA, a nicked mdRNA with three LNAs per sense strand fragment, and a single nucleotide gapped mdRNA with three LNAs per sense strand fragment. In other words, 660 different dsRNA were examined.

Briefly, multiwell plates were seeded with about 7-8×10⁵ HeLa cells/well in DMEM having 10% fetal bovine serum, and incubated overnight at 37° C./5% CO₂. The HeLa cell medium was changed to serum-free DMEM just prior to transfection. The psiCHECK™-2 vector, containing about a 1,000 basepair insert of a target gene, diluted in serum-free DMEM was mixed with diluted GenJet™ transfection reagent (SignalDT Biosystems, Hayward, Calif.) according to the manufacturer's instructions and then incubated at room temperature for 10 minutes. The GenJet/psiCHECK™-2-target gene solution was added to the HeLa cells and then incubated at 37° C., 5% CO₂ for 4.5 hours. After the vector transfection, cells were trypsinized and suspended in antibiotic-free DMEM containing 10% FBS at a concentration of 10⁵ cells per mL.

To transfect the dsRNA, the dsRNA was formulated in OPTI-MEM I reduced serum medium (Gibco® Invitrogen, Carlsbad, Calif.) and placed in multiwell plates. Then Lipofectamine™ RNAiMAX (Invitrogen) was mixed with OPTI-MEM per manufacture's specifications, added to each well containing dsRNA, mixed manually, and incubated at room temperature for 10-20 minutes. Then 30 μL of vector-transfected HeLa cells at 10⁵ cells per mL were added to each well (final dsRNA concentration of 25 nM), the plates were spun for 30 seconds at 1,000 rpm, and then incubated at 37° C./5% CO₂ for 2 days. The Cell Titer Blue (CTB) reagent (Promega, Madison, Wisconsin) was used to assay for cell viability and proliferation—none of the dsRNA showed any substantial toxicity.

After transfecting, the media and CTB reagent were removed and the wells washed once with 100 PBS. Cells were assayed for firefly and Renilla luciferase reporter activity by first adding Dual-Glo™ Luciferase Reagent (Promega, Madison, Wis.) for 10 minutes with shaking, and then quantitating the luminescent signal on a VICTOR³™ 1420 Multilabel Counter (PerkinElmer). After measuring the firefly luminescence, Stop & Glo® Reagent (Promega, Madison, Wis.) was added for 10 minutes with shaking to simultaneously quench the firefly reaction and initiate the Renilla luciferase reaction, which was then quantitated on a VICTOR³™ 1420 Multilabel Counter (PerkinElmer). The results are presented in Table 1.

TABLE 1
Gene Silencing Activity* of dsRNA Dicer Substrate and mdRNA (nicked or gapped) Dicer Substrate

| | | | | Dicer | | | Nicked | | | Gapped | | |
|---|---|---|---|---|---|---|---|---|---|---|---|---|
| | | | Dicer | Mean | Dicer | Nicked | Mean | Nicked | Gapped | Mean | Gapped | Length |
| Set | Target | Pos† | SEQ ID NOS‡ | (%) | 95% CI | SEQ ID NOS | (%) | 95% CI | SEQ ID NOS | (%) | 95% CI | 5′-S\hat{ } |
| 1 | AKT1 | 1862 | 63, 283 | 20.6 | 4.0% | 503, 723, 283 | 23.5 | 5.7% | 503, 940, 283 | 54.3 | 12.0% | 14 |
| 2 | AKT1 | 1883 | 64, 284 | 29.7 | 7.3% | 504, 724, 284 | 51.4 | 6.7% | 504, 941, 284 | 76.9 | 19.5% | 12 |
| 3 | AKT1 | 2178 | 65, 285 | 15.4 | 2.4% | 505, 725, 285 | 22.3 | 6.4% | 505, 942, 285 | 24.4 | 5.1% | 14 |
| 4 | AKT1 | 2199 | 66, 286 | 26.4 | 3.6% | 506, 726, 286 | 62.7 | 6.6% | 506, 943, 286 | 66.8 | 10.8% | 15 |
| 5 | AKT1 | 2264 | 67, 287 | 35.2 | 7.3% | 507, 727, 287 | 34.1 | 7.3% | 507, 944, 287 | 31.3 | 5.2% | 12 |
| 6 | AKT1 | 2580 | 68, 288 | 27.6 | 5.7% | 508, 728, 288 | 40.1 | 8.3% | 508, 945, 288 | 91.5 | 17.0% | 12 |
| 7 | AKT1 | 2606 | 69, 289 | 14.0 | 2.6% | 509, 729, 289 | 14.9 | 3.2% | 509, 946, 289 | 33.4 | 6.9% | 11 |
| 8 | AKT1 | 2629 | 70, 290 | 21.0 | 10.1% | 510, 730, 290 | 13.5 | 2.4% | 510, 947, 290 | 13.6 | 2.1% | 12 |
| 9 | AKT1 | 2661 | 71, 291 | 37.4 | 6.6% | 511, 731, 291 | 41.0 | 12.1% | 511, 948, 291 | 71.6 | 11.9% | 15 |
| 10 | AKT1 | 2663 | 72, 292 | 18.1 | 4.3% | 512, 732, 292 | 23.0 | 5.9% | 512, 949, 292 | 51.4 | 9.2% | 14 |
| 11 | BCR-ABL (b2a2) | 66 | 73, 293 | 16.9 | 5.9% | 513, 733, 293 | 30.4 | 10.5% | 513, 950, 293 | 38.2 | 11.7% | 13 |
| 12 | BCR-ABL (b2a2) | 190 | 74, 294 | 40.0 | 11.6% | 514, 734, 294 | 22.0 | 6.4% | 514, 951, 294 | 34.6 | 12.0% | 14 |
| 13 | BCR-ABL (b2a2) | 282 | 75, 295 | 24.2 | 5.2% | 515, 735, 295 | 37.6 | 8.2% | 515, 952, 295 | 34.6 | 8.6% | 13 |
| 14 | BCR-ABL (b2a2) | 284 | 76, 296 | 50.9 | 6.9% | 516, 736, 296 | 38.3 | 7.8% | 516, 953, 296 | 68.3 | 18.0% | 13 |
| 15 | BCR-ABL (b2a2) | 287 | 77, 297 | 45.5 | 13.2% | 517, 737, 297 | 39.6 | 11.5% | 517, 954, 297 | 75.2 | 17.2% | 14 |
| 16 | BCR-ABL (b2a2) | 289 | 78, 298 | 36.9 | 7.7% | 518, 738, 298 | 40.0 | 8.9% | 518, 955, 298 | 60.9 | 12.3% | 14 |
| 17 | BCR-ABL (b2a2) | 293 | 79, 299 | 55.9 | 9.8% | 519, 739, 299 | 58.6 | 14.7% | 519, 956, 299 | 87.0 | 14.3% | 13 |
| 18 | BCR-ABL (b2a2) | 461 | 80, 300 | 38.4 | 9.4% | 520, 740, 300 | 35.9 | 12.1% | 520, 957, 300 | 28.6 | 10.2% | 13 |
| 19 | BCR-ABL (b2a2) | 462 | 81, 301 | 31.1 | 13.7% | 521, 741, 301 | 26.5 | 5.5% | 521, 958, 301 | 35.8 | 10.7% | 14 |
| 20 | BCR-ABL (b2a2) | 561 | 82, 302 | 17.7 | 3.4% | 522, 742, 302 | 20.7 | 3.4% | 522, 959, 302 | 35.5 | 10.6% | 12 |
| 21 | BCR-ABL (b3a2) | 352 | 83, 303 | 45.4 | 7.0% | 523, 743, 303 | 39.8 | 8.3% | 523, 960, 303 | 45.5 | 11.0% | 12 |
| 22 | BCR-ABL (b3a2) | 353 | 84, 304 | 22.6 | 1.8% | 524, 744, 304 | 20.5 | 5.1% | 524, 961, 304 | 66.1 | 17.8% | 12 |
| 23 | BCR-ABL (b3a2) | 356 | 85, 305 | 11.9 | 2.5% | 525, 745, 305 | 28.4 | 5.8% | 525, 962, 305 | 56.0 | 10.6% | 13 |
| 24 | BCR-ABL (b3a2) | 357 | 86, 306 | 24.5 | 6.0% | 526, 746, 306 | 25.6 | 7.5% | 526, 963, 306 | 39.2 | 10.0% | 13 |
| 25 | BCR-ABL (b3a2) | 359 | 87, 307 | 56.8 | 9.3% | 527, 747, 307 | 42.4 | 7.3% | 527, 964, 307 | 46.4 | 9.5% | 13 |
| 26 | BCR-ABL (b3a2) | 360 | 88, 308 | 32.3 | 5.0% | 528, 748, 308 | 37.2 | 7.3% | 528, 965, 308 | 55.3 | 13.8% | 13 |
| 27 | BCR-ABL (b3a2) | 362 | 89, 309 | 12.4 | 3.2% | 529, 737, 309 | 26.3 | 9.8% | 529, 954, 309 | 46.2 | 8.3% | 14 |
| 28 | BCR-ABL (b3a2) | 410 | 90, 310 | 66.2 | 12.2% | 530, 749, 310 | 55.9 | 11.2% | 530, 966, 310 | 58.4 | 16.4% | 12 |
| 29 | BCR-ABL (b3a2) | 629 | 91, 311 | 35.0 | 11.7% | 531, 750, 311 | 46.5 | 10.1% | 531, 967, 311 | 41.0 | 9.0% | 13 |
| 30 | BCR-ABL (b3a2) | 727 | 92, 312 | 83.4 | 13.6% | 532, 751, 312 | 76.7 | 22.5% | 532, 968, 312 | 62.9 | 10.9% | 12 |
| 31 | EGFR | 4715 | 93, 313 | 15.3 | 2.2% | 533, 752, 313 | 9.4 | 0.9% | 533, 969, 313 | 11.3 | 1.7% | 11 |
| 32 | EGFR | 4759 | 94, 314 | 3.8 | 0.4% | 534, 753, 314 | 6.3 | 0.8% | 534, 970, 314 | 8.4 | 1.1% | 12 |
| 33 | EGFR | 4810 | 95, 315 | 5.2 | 0.6% | 535, 754, 315 | 5.8 | 0.7% | 535, 971, 315 | 7.2 | 1.0% | 13 |
| 34 | EGFR | 5249 | 96, 316 | 2.6 | 0.4% | 536, 755, 316 | 16.6 | 1.8% | 536, 972, 316 | 42.9 | 3.5% | 14 |
| 35 | EGFR | 5279 | 97, 317 | 7.6 | 1.0% | 537, 756, 317 | 10.6 | 1.1% | 537, 973, 317 | 11.8 | 1.7% | 13 |
| 36 | EGFR | 5374 | 98, 318 | 9.6 | 1.0% | 538, 757, 318 | 8.7 | 0.9% | 538, 974, 318 | 34.7 | 4.3% | 12 |
| 37 | EGFR | 5442 | 99, 319 | 4.1 | 0.8% | 539, 758, 319 | 15.1 | 1.8% | 539, 975, 319 | 19.7 | 2.4% | 12 |
| 38 | EGFR | 5451 | 100, 320 | 5.1 | 0.3% | 540, 759, 320 | 11.5 | 1.3% | 540, 976, 320 | 16.5 | 3.0% | 13 |
| 39 | EGFR | 5469 | 101, 321 | 5.6 | 0.8% | 541, 760, 321 | 5.1 | 0.5% | 541, 977, 321 | 12.2 | 2.5% | 13 |
| 40 | EGFR | 5483 | 102, 322 | 2.2 | 0.4% | 542, 761, 322 | 2.4 | 0.5% | 542, 978, 322 | 6.1 | 0.7% | 9 |
| 41 | FLT1 | 863 | 103, 323 | 7.6 | 1.1% | 543, 762, 323 | 10.2 | 3.3% | 543, 979, 323 | 29.2 | 8.1% | 12 |
| 42 | FLT1 | 906 | 104, 324 | 10.0 | 2.4% | 544, 763, 324 | 10.8 | 0.8% | 544, 980, 324 | 12.4 | 2.1% | 12 |
| 43 | FLT1 | 993 | 105, 325 | 12.2 | 2.5% | 545, 764, 325 | 13.7 | 2.8% | 545, 981, 325 | 20.0 | 11.3% | 13 |
| 44 | FLT1 | 1283 | 106, 326 | 19.6 | 4.5% | 546, 765, 326 | 25.8 | 7.3% | 546, 982, 326 | 18.7 | 6.5% | 12 |
| 45 | FLT1 | 1289 | 107, 327 | 15.5 | 2.0% | 547, 766, 327 | 13.5 | 1.6% | 547, 983, 327 | 22.5 | 5.0% | 12 |
| 46 | FLT1 | 1349 | 108, 328 | 36.8 | 4.2% | 548, 767, 328 | 22.9 | 4.0% | 548, 984, 328 | 52.7 | 5.4% | 14 |
| 47 | FLT1 | 1354 | 109, 329 | 36.6 | 4.0% | 549, 768, 329 | 49.7 | 5.9% | 549, 985, 329 | 45.8 | 9.3% | 14 |
| 48 | FLT1 | 1448 | 110, 330 | 9.3 | 2.5% | 550, 769, 330 | 16.1 | 2.9% | 550, 986, 330 | 24.2 | 3.6% | 13 |
| 49 | FLT1 | 1459 | 111, 331 | 13.7 | 3.6% | 551, 770, 331 | 20.0 | 8.7% | 551, 987, 331 | 22.4 | 4.4% | 12 |
| 50 | FLT1 | 1700 | 112, 332 | 7.9 | 2.2% | 552, 771, 332 | 11.2 | 3.7% | 552, 988, 332 | 36.4 | 8.0% | 13 |
| 51 | FRAP1 | 7631 | 113, 333 | 9.5 | 2.7% | 553, 772, 333 | 23.3 | 4.9% | 553, 989, 333 | 61.8 | 18.3% | 13 |
| 52 | FRAP1 | 7784 | 114, 334 | 15.1 | 1.7% | 554, 773, 334 | 19.9 | 2.8% | 554, 990, 334 | 29.3 | 3.4% | 12 |
| 53 | FRAP1 | 7812 | 115, 335 | 11.9 | 2.9% | 555, 774, 335 | 14.4 | 3.2% | 555, 991, 335 | 28.3 | 12.7% | 11 |
| 54 | FRAP1 | 7853 | 116, 336 | 16.8 | 3.3% | 556, 775, 336 | 24.1 | 3.7% | 556, 992, 336 | 67.5 | 9.2% | 11 |
| 55 | FRAP1 | 8018 | 117, 337 | 41.1 | 9.1% | 557, 776, 337 | 19.8 | 3.3% | 557, 993, 337 | 41.8 | 9.6% | 12 |
| 56 | FRAP1 | 8102 | 118, 338 | 35.7 | 5.1% | 558, 777, 338 | 30.2 | 6.3% | 558, 994, 338 | 39.5 | 9.9% | 12 |
| 57 | FRAP1 | 8177 | 119, 339 | 21.2 | 3.9% | 559, 778, 339 | 33.2 | 9.3% | 559, 995, 339 | 47.3 | 12.3% | 14 |
| 58 | FRAP1 | 8348 | 120, 340 | 25.8 | 3.6% | 560, 779, 340 | 26.8 | 4.4% | 560, 996, 340 | 37.4 | 4.7% | 11 |
| 59 | FRAP1 | 8435 | 121, 341 | 41.1 | 6.7% | 561, 780, 341 | 54.1 | 9.5% | 561, 997, 341 | 74.9 | 8.5% | 12 |
| 60 | FRAP1 | 8542 | 122, 342 | 23.1 | 4.8% | 562, 781, 342 | 16.5 | 5.5% | 562, 998, 342 | 33.6 | 6.4% | 10 |
| 61 | HIF1A | 1780 | 123, 343 | 76.6 | 14.9% | 563, 782, 343 | 89.2 | 11.9% | 563, 999, 343 | 86.3 | 9.3% | 12 |
| 62 | HIF1A | 1831 | 124, 344 | 9.0 | 0.6% | 564, 783, 344 | 14.0 | 2.3% | 564, 1000, 344 | 38.2 | 8.5% | 12 |
| 63 | HIF1A | 1870 | 125, 345 | 21.4 | 4.5% | 565, 784, 345 | 21.2 | 3.3% | 565, 1001, 345 | 19.6 | 2.2% | 13 |
| 64 | HIF1A | 1941 | 126, 346 | 8.9 | 2.1% | 566, 785, 346 | 11.4 | 2.2% | 566, 1002, 346 | 11.7 | 2.5% | 12 |
| 65 | HIF1A | 2068 | 127, 347 | 7.8 | 1.5% | 567, 786, 347 | 7.0 | 1.4% | 567, 1003, 347 | 16.9 | 3.9% | 12 |
| 66 | HIF1A | 2133 | 128, 348 | 13.0 | 2.0% | 568, 787, 348 | 16.7 | 3.1% | 568, 1004, 348 | 16.3 | 3.1% | 10 |
| 67 | HIF1A | 2232 | 129, 349 | 8.6 | 2.0% | 569, 788, 349 | 17.4 | 3.6% | 569, 1005, 349 | 37.8 | 9.6% | 13 |
| 68 | HIF1A | 2273 | 130, 350 | 19.1 | 5.3% | 570, 789, 350 | 23.4 | 4.4% | 570, 1006, 350 | 20.3 | 3.4% | 12 |
| 69 | HIF1A | 2437 | 131, 351 | 8.2 | 1.4% | 571, 790, 351 | 47.7 | 11.5% | 571, 1007, 351 | 72.4 | 14.3% | 13 |
| 70 | HIF1A | 2607 | 132, 352 | 8.0 | 2.1% | 572, 791, 352 | 11.0 | 1.2% | 572, 1008, 352 | 33.6 | 6.0% | 13 |
| 71 | IL17A | 923 | 133, 353 | 5.0 | 0.6% | 573, 792, 353 | 7.3 | 0.7% | 573, 1009, 353 | 26.3 | 2.5% | 12 |
| 72 | IL17A | 962 | 134, 354 | 6.7 | 0.8% | 574, 793, 354 | 7.7 | 0.9% | 574, 1010, 354 | 8.9 | 2.0% | 13 |
| 73 | IL17A | 969 | 135, 355 | 8.9 | 1.7% | 575, 794, 355 | 17.1 | 1.6% | 575, 1011, 355 | 49.5 | 4.3% | 14 |
| 74 | IL17A | 1098 | 136, 356 | 7.2 | 1.3% | 576, 795, 356 | 10.0 | 2.4% | 576, 1012, 356 | 15.4 | 2.8% | 12 |
| 75 | IL17A | 1201 | 137, 357 | 14.1 | 2.2% | 577, 796, 357 | 13.4 | 1.1% | 577, 1013, 357 | 17.2 | 2.8% | 12 |
| 76 | IL17A | 1433 | 138, 358 | 107.1 | 9.7% | 578, 797, 358 | 111.5 | 10.4% | 578, 1014, 358 | 108.1 | 8.8% | 13 |
| 77 | IL17A | 1455 | 139, 359 | 115.4 | 11.1% | 579, 798, 359 | 120.8 | 8.7% | 579, 1015, 359 | 120.3 | 9.9% | 12 |
| 78 | IL17A | 1478 | 140, 360 | 82.7 | 6.3% | 580, 799, 360 | 87.6 | 5.0% | 580, 1016, 360 | 95.9 | 5.6% | 14 |
| 79 | IL17A | 1663 | 141, 361 | 140.2 | 7.8% | 581, 800, 361 | 125.9 | 9.8% | 581, 1017, 361 | 114.7 | 10.1% | 14 |
| 80 | IL17A | 1764 | 142, 362 | 114.3 | 9.2% | 582, 801, 362 | 109.4 | 2.9% | 582, 1018, 362 | 105.7 | 8.1% | 15 |
| 81 | IL18 | 210 | 143, 363 | 13.8 | 2.8% | 583, 802, 363 | 23.9 | 5.8% | 583, 1019, 363 | 21.4 | 5.7% | 14 |
| 82 | IL18 | 368 | 144, 364 | 22.5 | 1.8% | 584, 803, 364 | 21.0 | 2.0% | 584, 1020, 364 | 29.7 | 3.7% | 13 |
| 83 | IL18 | 479 | 145, 365 | 88.1 | 12.9% | 585, 804, 365 | 66.3 | 9.8% | 585, 1021, 365 | 80.0 | 16.8% | 14 |
| 84 | IL18 | 508 | 146, 366 | 8.0 | 1.9% | 586, 805, 366 | 15.7 | 3.5% | 586, 1022, 366 | 17.0 | 5.7% | 12 |
| 85 | IL18 | 521 | 147, 367 | 9.9 | 2.1% | 587, 806, 367 | 10.8 | 2.1% | 587, 1023, 367 | 18.4 | 3.3% | 11 |
| 86 | IL18 | 573 | 148, 368 | 18.6 | 4.7% | 588, 807, 368 | 24.8 | 7.6% | 588, 1024, 368 | 48.8 | 7.7% | 14 |
| 87 | IL18 | 605 | 149, 369 | 27.5 | 6.1% | 589, 808, 369 | 21.3 | 3.9% | 589, 1025, 369 | 14.9 | 2.7% | 13 |
| 88 | IL18 | 663 | 150, 370 | 5.3 | 1.0% | 590, 809, 370 | 8.2 | 1.5% | 590, 1026, 370 | 11.7 | 3.4% | 12 |
| 89 | IL18 | 785 | 151, 371 | 8.6 | 1.0% | 591, 810, 371 | 11.7 | 2.8% | 591, 1027, 371 | 21.1 | 9.1% | 12 |
| 90 | IL18 | 918 | 152, 372 | 13.9 | 1.6% | 592, 811, 372 | 15.0 | 3.0% | 592, 1028, 372 | 30.4 | 3.6% | 11 |
| 91 | IL6 | 24 | 153, 373 | 22.6 | 1.7% | 593, 812, 373 | 45.7 | 7.8% | 593, 1029, 373 | 47.8 | 4.5% | 13 |
| 92 | IL6 | 74 | 154, 374 | 52.5 | 12.6% | 594, 813, 374 | 56.4 | 7.1% | 594, 1030, 374 | 88.3 | 15.5% | 12 |
| 93 | IL6 | 160 | 155, 375 | 49.8 | 7.8% | 595, 814, 375 | 50.6 | 6.1% | 595, 1031, 375 | 68.3 | 9.4% | 14 |
| 94 | IL6 | 370 | 156, 376 | 44.7 | 8.2% | 596, 815, 376 | 52.5 | 4.2% | 596, 1032, 376 | 74.3 | 9.3% | 13 |
| 95 | IL6 | 451 | 157, 377 | 39.3 | 5.0% | 597, 816, 377 | 35.6 | 4.1% | 597, 1033, 377 | 66.6 | 7.1% | 13 |
| 96 | IL6 | 481 | 158, 378 | 68.3 | 8.1% | 598, 817, 378 | 78.7 | 15.6% | 598, 1034, 378 | 63.2 | 6.2% | 11 |
| 97 | IL6 | 710 | 159, 379 | 29.2 | 4.2% | 599, 818, 379 | 32.0 | 4.1% | 599, 1035, 379 | 77.3 | 11.4% | 12 |
| 98 | IL6 | 822 | 160, 380 | 73.7 | 11.0% | 600, 819, 380 | 72.2 | 11.6% | 600, 1036, 380 | 85.2 | 13.3% | 12 |
| 99 | IL6 | 836 | 161, 381 | 98.8 | 21.8% | 601, 820, 381 | 95.0 | 13.2% | 601, 1037, 381 | 90.5 | 15.6% | 13 |
| 100 | IL6 | 960 | 162, 382 | 31.1 | 4.4% | 602, 821, 382 | 20.5 | 6.1% | 602, 1038, 382 | 25.6 | 2.4% | 12 |
| 101 | MAP2K1 | 1237 | 163, 383 | 21.0 | 3.3% | 603, 822, 383 | 27.9 | 3.8% | 603, 1039, 383 | 50.0 | 8.8% | 11 |
| 102 | MAP2K1 | 1342 | 164, 384 | 3.9 | 0.5% | 604, 823, 384 | 8.7 | 1.5% | 604, 1040, 384 | 11.4 | 1.3% | 13 |
| 103 | MAP2K1 | 1501 | 165, 385 | 12.9 | 1.9% | 605, 824, 385 | 19.4 | 2.9% | 605, 1041, 385 | 19.7 | 5.3% | 12 |
| 104 | MAP2K1 | 1542 | 166, 386 | 7.2 | 1.3% | 606, 825, 386 | 11.7 | 2.1% | 606, 1042, 386 | 18.7 | 3.2% | 11 |
| 105 | MAP2K1 | 1544 | 167, 387 | 13.1 | 2.1% | 607, 826, 387 | 11.1 | 1.1% | 607, 1043, 387 | 16.5 | 3.0% | 10 |
| 106 | MAP2K1 | 1728 | 168, 388 | 11.9 | 1.7% | 608, 827, 388 | 11.9 | 1.0% | 608, 1044, 388 | 27.9 | 4.3% | 13 |
| 107 | MAP2K1 | 1777 | 169, 389 | 18.3 | 2.8% | 609, 828, 389 | 37.2 | 4.3% | 609, 1045, 389 | 64.5 | 8.5% | 13 |
| 108 | MAP2K1 | 1892 | 170, 390 | 34.5 | 4.7% | 610, 829, 390 | 37.6 | 6.8% | 610, 1046, 390 | 42.4 | 7.3% | 12 |
| 109 | MAP2K1 | 1954 | 171, 391 | 4.6 | 0.5% | 611, 830, 391 | 4.2 | 0.5% | 611, 1047, 391 | 6.5 | 1.1% | 13 |
| 110 | MAP2K1 | 2062 | 172, 392 | 10.2 | 0.8% | 612, 831, 392 | 10.4 | 2.9% | 612, 1048, 392 | 12.2 | 2.0% | 12 |
| 111 | MAPK1 | 3683 | 173, 393 | 7.0 | 0.9% | 613, 614, 393 | 24.4 | 17.3% | 613, 1049, 393 | 25.2 | 2.6% | 12 |
| 112 | MAPK1 | 3695 | 174, 394 | 32.9 | 4.6% | 614, 832, 394 | 30.9 | 4.0% | 614, 1050, 394 | 33.8 | 3.1% | 13 |
| 113 | MAPK1 | 3797 | 175, 395 | 7.4 | 1.1% | 615, 833, 395 | 6.4 | 1.3% | 615, 1051, 395 | 40.4 | 5.8% | 11 |
| 114 | MAPK1 | 3905 | 176, 396 | 8.0 | 1.0% | 616, 834, 396 | 8.1 | 0.5% | 616, 1052, 396 | 14.8 | 1.4% | 12 |
| 115 | MAPK1 | 3916 | 177, 397 | 11.0 | 1.7% | 617, 835, 397 | 16.0 | 3.3% | 617, 1053, 397 | 45.5 | 8.1% | 10 |
| 116 | MAPK1 | 3943 | 178, 398 | 6.8 | 0.8% | 618, 836, 398 | 6.6 | 0.7% | 618, 1054, 398 | 11.0 | 2.3% | 10 |
| 117 | MAPK1 | 4121 | 179, 399 | 7.6 | 1.1% | 619, 837, 399 | 12.7 | 1.6% | 619, 1055, 399 | 25.1 | 3.1% | 12 |
| 118 | MAPK1 | 4256 | 180, 400 | 27.6 | 2.5% | 620, 838, 400 | 36.8 | 4.0% | 620, 1056, 400 | 57.7 | 7.0% | 13 |
| 119 | MAPK1 | 4294 | 181, 401 | 31.0 | 3.0% | 621, 839, 401 | 22.3 | 3.6% | 621, 1057, 401 | 50.9 | 4.6% | 12 |
| 120 | MAPK1 | 4375 | 182, 402 | 10.9 | 1.1% | 622, 840, 402 | 12.4 | 1.4% | 622, 1058, 402 | 16.9 | 2.7% | 11 |
| 121 | MAPK14 | 2715 | 183, 403 | 11.4 | 2.8% | 623, 841, 403 | 16.5 | 4.1% | 623, 1059, 403 | 16.6 | 2.4% | 12 |
| 122 | MAPK14 | 2737 | 184, 404 | 7.5 | 0.8% | 624, 842, 404 | 10.3 | 1.1% | 624, 1060, 404 | 13.1 | 1.2% | 11 |
| 123 | MAPK14 | 2750 | 185, 405 | 8.7 | 1.0% | 625, 843, 405 | 12.2 | 1.8% | 625, 1061, 405 | 15.8 | 1.9% | 13 |
| 124 | MAPK14 | 2817 | 186, 406 | 6.4 | 0.8% | 626, 844, 406 | 14.6 | 1.7% | 626, 1062, 406 | 19.4 | 2.0% | 11 |
| 125 | MAPK14 | 3091 | 187, 407 | 9.9 | 0.6% | 627, 845, 407 | 10.3 | 1.3% | 627, 1063, 407 | 24.7 | 1.5% | 11 |
| 126 | MAPK14 | 3312 | 188, 408 | 20.4 | 1.8% | 628, 846, 408 | 30.5 | 2.9% | 628, 1064, 408 | 38.5 | 3.4% | 13 |
| 127 | MAPK14 | 3346 | 189, 409 | 20.9 | 1.6% | 629, 847, 409 | 23.0 | 2.6% | 629, 1065, 409 | 58.3 | 6.7% | 11 |
| 128 | MAPK14 | 3531 | 190, 410 | 42.4 | 3.2% | 630, 848, 410 | 55.1 | 5.0% | 630, 1066, 410 | 61.9 | 3.6% | 12 |
| 129 | MAPK14 | 3621 | 191, 411 | 28.6 | 1.9% | 631, 849, 411 | 42.4 | 13.5% | 631, 1067, 411 | 71.9 | 5.2% | 11 |
| 130 | MAPK14 | 3680 | 192, 412 | 15.6 | 1.3% | 632, 850, 412 | 15.5 | 1.9% | 632, 1068, 412 | 19.8 | 2.1% | 12 |
| 131 | PDGFA | 1322 | 193, 413 | 23.7 | 3.6% | 633, 851, 413 | 31.6 | 4.3% | 633, 1069, 413 | 38.4 | 3.3% | 12 |
| 132 | PDGFA | 1332 | 194, 414 | 35.5 | 5.4% | 634, 852, 414 | 48.4 | 3.0% | 634, 1070, 414 | 65.4 | 10.5% | 14 |
| 133 | PDGFA | 1395 | 195, 415 | 25.9 | 3.3% | 635, 853, 415 | 40.2 | 6.0% | 635, 1071, 415 | 55.2 | 9.8% | 14 |
| 134 | PDGFA | 1669 | 196, 416 | 40.4 | 5.1% | 636, 854, 416 | 29.5 | 4.3% | 636, 1072, 416 | 33.9 | 5.9% | 12 |
| 135 | PDGFA | 1676 | 197, 417 | 27.1 | 2.5% | 637, 855, 417 | 36.8 | 4.5% | 637, 1073, 417 | 47.4 | 3.4% | 13 |
| 136 | PDGFA | 1748 | 198, 418 | 27.4 | 4.7% | 638, 856, 418 | 34.5 | 5.0% | 638, 1074, 418 | 47.5 | 4.7% | 11 |
| 137 | PDGFA | 2020 | 199, 419 | 31.6 | 6.6% | 639, 857, 419 | 37.5 | 4.3% | 639, 1075, 419 | 51.9 | 5.0% | 13 |
| 138 | PDGFA | 2021 | 200, 420 | 16.7 | 1.0% | 640, 858, 420 | 24.2 | 3.1% | 640, 1076, 420 | 62.6 | 6.9% | 14 |
| 139 | PDGFA | 2030 | 201, 421 | 38.7 | 6.2% | 641, 859, 421 | 47.0 | 10.5% | 641, 1077, 421 | 80.5 | 7.6% | 13 |
| 140 | PDGFA | 2300 | 202, 422 | 55.3 | 7.7% | 642, 860, 422 | 41.2 | 4.7% | 642, 1078, 422 | 71.7 | 9.1% | 15 |
| 141 | PDGFRA | 4837 | 203, 423 | 16.9 | 3.1% | 643, 861, 423 | 21.1 | 5.1% | 643, 1079, 423 | 23.1 | 4.8% | 12 |
| 142 | PDGFRA | 4900 | 204, 424 | 23.8 | 3.8% | 644, 862, 424 | 40.9 | 8.4% | 644, 1080, 424 | 62.5 | 12.5% | 16 |
| 143 | PDGFRA | 5007 | 205, 425 | 52.6 | 9.4% | 645, 863, 425 | 49.6 | 7.7% | 645, 1081, 425 | 47.0 | 9.5% | 12 |
| 144 | PDGFRA | 5043 | 206, 426 | 30.1 | 7.9% | 646, 864, 426 | 30.0 | 5.4% | 646, 1082, 426 | 57.3 | 7.8% | 11 |
| 145 | PDGFRA | 5082 | 207, 427 | 8.3 | 1.1% | 647, 865, 427 | 11.9 | 1.8% | 647, 1083, 427 | 18.2 | 4.0% | 13 |
| 146 | PDGFRA | 5352 | 208, 428 | 6.3 | 1.4% | 648, 866, 428 | 8.2 | 1.6% | 648, 1084, 428 | 7.9 | 1.1% | 12 |
| 147 | PDGFRA | 5367 | 209, 429 | 19.1 | 5.6% | 649, 867, 429 | 10.9 | 1.6% | 649, 1085, 429 | 25.1 | 2.9% | 14 |
| 148 | PDGFRA | 5496 | 210, 430 | 18.9 | 5.4% | 650, 868, 430 | 17.0 | 2.9% | 650, 1086, 430 | 17.8 | 4.0% | 12 |
| 149 | PDGFRA | 5706 | 211, 431 | 24.5 | 4.0% | 651, 869, 431 | 47.8 | 4.3% | 651, 1087, 431 | 50.6 | 5.5% | 13 |
| 150 | PDGFRA | 5779 | 212, 432 | 13.0 | 1.4% | 652, 870, 432 | 14.0 | 2.1% | 652, 1088, 432 | 17.2 | 4.3% | 14 |
| 151 | PIK3CA | 213 | 213, 433 | 4.3 | 1.0% | 653, 871, 433 | 3.7 | 0.6% | 653, 1089, 433 | 5.7 | 0.9% | 12 |
| 152 | PIK3CA | 389 | 214, 434 | 5.3 | 1.0% | 654, 872, 434 | 7.0 | 1.5% | 654, 1090, 434 | 5.6 | 1.5% | 10 |
| 153 | PIK3CA | 517 | 215, 435 | 9.6 | 1.1% | 655, 873, 435 | 11.5 | 2.1% | 655, 1091, 435 | 13.5 | 1.6% | 11 |
| 154 | PIK3CA | 630 | 216, 436 | 6.1 | 1.2% | 656, 874, 436 | 8.9 | 2.6% | 656, 1092, 436 | 9.3 | 1.8% | 12 |
| 155 | PIK3CA | 680 | 217, 437 | 3.8 | 0.3% | 657, 875, 437 | 5.9 | 0.6% | 657, 1093, 437 | 6.9 | 1.0% | 11 |
| 156 | PIK3CA | 732 | 218, 438 | 5.7 | 1.7% | 658, 876, 438 | 15.3 | 1.5% | 658, 1094, 438 | 17.4 | 4.0% | 11 |
| 157 | PIK3CA | 736 | 219, 439 | 5.9 | 0.9% | 659, 877, 439 | 7.8 | 1.1% | 659, 1095, 439 | 6.5 | 1.4% | 12 |
| 158 | PIK3CA | 923 | 220, 440 | 5.0 | 0.7% | 660, 878, 440 | 8.5 | 1.5% | 660, 1096, 440 | 7.4 | 0.6% | 12 |
| 159 | PIK3CA | 1087 | 221, 441 | 8.1 | 2.3% | 661, 879, 441 | 8.5 | 1.6% | 661, 1097, 441 | 17.5 | 4.9% | 12 |
| 160 | PIK3CA | 1094 | 222, 442 | 13.0 | 3.8% | 662, 880, 442 | 13.0 | 2.5% | 662, 1098, 442 | 30.1 | 6.4% | 11 |
| 161 | PKN3 | 2408 | 223, 443 | 9.4 | 2.1% | 663, 881, 443 | 15.2 | 3.7% | 663, 665, 443 | 32.1 | 6.6% | 12 |
| 162 | PKN3 | 2420 | 224, 444 | 14.5 | 1.7% | 664, 882, 444 | 30.4 | 7.5% | 664, 1099, 444 | 40.1 | 6.7% | 12 |
| 163 | PKN3 | 2421 | 225, 445 | 15.2 | 2.0% | 665, 883, 445 | 20.6 | 2.7% | 665, 1100, 445 | 50.8 | 7.8% | 12 |
| 164 | PKN3 | 2425 | 226, 446 | 28.4 | 3.8% | 666, 884, 446 | 27.0 | 6.9% | 666, 1101, 446 | 36.2 | 4.8% | 15 |
| 165 | PKN3 | 2682 | 227, 447 | 30.0 | 4.6% | 667, 885, 447 | 27.1 | 2.8% | 667, 1102, 447 | 37.1 | 6.2% | 11 |
| 166 | PKN3 | 2683 | 228, 448 | 22.4 | 2.8% | 668, 886, 448 | 34.8 | 2.2% | 668, 1103, 448 | 51.9 | 7.4% | 12 |
| 167 | PKN3 | 2931 | 229, 449 | 35.1 | 4.4% | 669, 887, 449 | 57.3 | 7.8% | 669, 1104, 449 | 88.6 | 7.1% | 13 |
| 168 | PKN3 | 3063 | 230, 450 | 21.8 | 3.1% | 670, 888, 450 | 28.6 | 8.5% | 670, 1105, 450 | 40.5 | 6.2% | 12 |
| 169 | PKN3 | 3314 | 231, 451 | 9.7 | 1.8% | 671, 889, 451 | 12.0 | 1.4% | 671, 1106, 451 | 17.3 | 1.3% | 10 |
| 170 | PKN3 | 3315 | 232, 452 | 10.1 | 1.3% | 672, 890, 452 | 15.3 | 2.8% | 672, 1107, 452 | 37.4 | 3.6% | 11 |
| 171 | RAF1 | 1509 | 233, 453 | 46.2 | 9.4% | 673, 891, 453 | 51.3 | 10.7% | 673, 1108, 453 | 61.3 | 4.4% | 12 |
| 172 | RAF1 | 1512 | 234, 454 | 40.1 | 9.7% | 674, 892, 454 | 34.5 | 5.6% | 674, 1109, 454 | 62.4 | 8.6% | 13 |
| 173 | RAF1 | 1628 | 235, 455 | 48.3 | 7.9% | 675, 893, 455 | 47.4 | 7.1% | 675, 1110, 455 | 41.1 | 5.1% | 12 |
| 174 | RAF1 | 1645 | 236, 456 | 38.9 | 2.3% | 676, 894, 456 | 62.1 | 9.0% | 676, 1111, 456 | 85.0 | 9.3% | 13 |
| 175 | RAF1 | 1780 | 237, 457 | 22.6 | 4.9% | 677, 895, 457 | 24.8 | 5.3% | 677, 1112, 457 | 37.6 | 10.4% | 12 |
| 176 | RAF1 | 1799 | 238, 458 | 23.2 | 3.1% | 678, 896, 458 | 43.6 | 7.6% | 678, 1113, 458 | 50.7 | 6.2% | 12 |
| 177 | RAF1 | 1807 | 239, 459 | 28.0 | 5.4% | 679, 897, 459 | 34.8 | 5.8% | 679, 1114, 459 | 37.0 | 5.3% | 15 |
| 178 | RAF1 | 1863 | 240, 460 | 28.2 | 3.1% | 680, 898, 460 | 38.1 | 4.5% | 680, 1115, 460 | 35.7 | 4.2% | 14 |
| 179 | RAF1 | 2157 | 241, 461 | 68.8 | 6.5% | 681, 899, 461 | 64.1 | 8.0% | 681, 1116, 461 | 86.7 | 12.6% | 14 |
| 180 | RAF1 | 2252 | 242, 462 | 11.4 | 1.7% | 682, 900, 462 | 25.8 | 5.4% | 682, 1117, 462 | 71.2 | 10.7% | 13 |
| 181 | SRD5A1 | 1150 | 243, 463 | 3.7 | 0.5% | 683, 901, 463 | 4.4 | 0.7% | 683, 1118, 463 | 3.8 | 0.4% | 12 |
| 182 | SRD5A1 | 1153 | 244, 464 | 3.2 | 0.4% | 684, 902, 464 | 5.2 | 0.5% | 684, 1119, 464 | 7.0 | 0.9% | 12 |
| 183 | SRD5A1 | 1845 | 245, 465 | 3.9 | 0.5% | 685, 903, 465 | 4.5 | 0.6% | 685, 1120, 465 | 7.4 | 0.8% | 13 |
| 184 | SRD5A1 | 1917 | 246, 466 | 9.4 | 0.8% | 686, 904, 466 | 10.2 | 1.3% | 686, 1121, 466 | 22.0 | 2.8% | 12 |
| 185 | SRD5A1 | 1920 | 247, 467 | 4.6 | 0.3% | 687, 905, 467 | 4.9 | 1.0% | 687, 1122, 467 | 6.4 | 0.5% | 11 |
| 186 | SRD5A1 | 1964 | 248, 468 | 6.2 | 0.7% | 688, 906, 468 | 10.4 | 0.7% | 688, 1123, 468 | 21.0 | 4.6% | 10 |
| 187 | SRD5A1 | 1981 | 249, 469 | 6.5 | 1.0% | 689, 907, 469 | 7.1 | 0.7% | 689, 1124, 469 | 8.8 | 1.5% | 12 |
| 188 | SRD5A1 | 2084 | 250, 470 | 16.9 | 1.1% | 690, 908, 470 | 15.7 | 1.5% | 690, 1125, 470 | 13.3 | 1.5% | 12 |
| 189 | SRD5A1 | 2085 | 251, 471 | 17.3 | 1.6% | 691, 909, 471 | 19.4 | 1.7% | 691, 1126, 471 | 20.8 | 2.6% | 12 |
| 190 | SRD5A1 | 2103 | 252, 472 | 7.5 | 1.3% | 692, 910, 472 | 10.9 | 1.2% | 692, 1127, 472 | 12.3 | 1.7% | 12 |
| 191 | TNF | 32 | 253, 473 | 71.4 | 13.2% | 693, 911, 473 | 93.7 | 14.9% | 693, 1128, 473 | 122.6 | 21.1% | 12 |
| 192 | TNF | 649 | 254, 474 | 100.0 | 16.3% | 694, 912, 474 | 127.7 | 12.6% | 694, 1129, 474 | 147.9 | 21.7% | 12 |
| 193 | TNF | 802 | 255, 475 | 67.2 | 10.7% | 695, 913, 475 | 64.0 | 6.6% | 695, 1130, 475 | 116.4 | 21.0% | 12 |
| 194 | TNF | 875 | 256, 476 | 101.7 | 19.9% | 696, 914, 476 | 99.3 | 15.5% | 696, 1131, 476 | 108.8 | 14.2% | 12 |
| 195 | TNF | 983 | 257, 477 | 94.5 | 7.0% | 697, 915, 477 | 83.1 | 7.3% | 697, 1132, 477 | 140.6 | 20.4% | 11 |
| 196 | TNF | 987 | 258, 478 | 82.0 | 10.9% | 698, 916, 478 | 139.4 | 8.2% | 698, 1133, 478 | 143.8 | 9.2% | 10 |
| 197 | TNF | 992 | 259, 479 | 126.7 | 15.8% | 699, 700, 479 | 121.7 | 10.8% | 699, 1134, 479 | 115.9 | 16.4% | 11 |
| 198 | TNF | 1003 | 260, 480 | 123.4 | 16.7% | 700, 917, 480 | 114.4 | 47.8% | 700, 1135, 480 | 98.5 | 17.2% | 14 |
| 199 | TNF | 1630 | 261, 481 | 58.0 | 5.7% | 701, 918, 481 | 56.1 | 9.4% | 701, 1136, 481 | 71.0 | 17.2% | 11 |
| 200 | TNF | 1631 | 262, 482 | 54.2 | 13.4% | 702, 919, 482 | 63.9 | 10.1% | 702, 1137, 482 | 73.8 | 14.8% | 11 |
| 201 | TNFSF13B | 188 | 263, 483 | 20.4 | 3.2% | 703, 920, 483 | 46.2 | 11.9% | 703, 1138, 483 | 58.4 | 12.7% | 13 |
| 202 | TNFSF13B | 313 | 264, 484 | 15.9 | 5.1% | 704, 921, 484 | 18.9 | 7.4% | 704, 1139, 484 | 48.0 | 8.1% | 12 |
| 203 | TNFSF13B | 337 | 265, 485 | 22.3 | 4.6% | 705, 922, 485 | 37.1 | 11.0% | 705, 1140, 485 | 63.6 | 10.4% | 12 |
| 204 | TNFSF13B | 590 | 266, 486 | 35.8 | 8.7% | 706, 923, 486 | 49.4 | 11.0% | 706, 1141, 486 | 50.7 | 10.3% | 10 |
| 205 | TNFSF13B | 652 | 267, 487 | 21.3 | 7.2% | 707, 924, 487 | 57.6 | 16.7% | 707, 1142, 487 | 78.8 | 5.6% | 14 |
| 206 | TNFSF13B | 661 | 268, 488 | 28.8 | 3.0% | 708, 925, 488 | 38.3 | 8.4% | 708, 1143, 488 | 56.5 | 16.3% | 12 |
| 207 | TNFSF13B | 684 | 269, 489 | 46.3 | 7.2% | 709, 926, 489 | 43.8 | 9.7% | 709, 1144, 489 | 54.5 | 4.6% | 12 |
| 208 | TNFSF13B | 905 | 270, 490 | 18.5 | 5.0% | 710, 927, 490 | 27.9 | 3.1% | 710, 1145, 490 | 51.7 | 10.9% | 12 |
| 209 | TNFSF13B | 961 | 271, 491 | 21.4 | 4.0% | 711, 928, 491 | 37.5 | 10.1% | 711, 1146, 491 | 77.6 | 11.2% | 14 |
| 210 | TNFSF13B | 1150 | 272, 492 | 24.1 | 7.0% | 712, 929, 492 | 23.4 | 5.7% | 712, 1147, 492 | 35.9 | 8.0% | 13 |
| 211 | VEGFA | 1426 | 273, 493 | 14.5 | 2.2% | 713, 930, 493 | 18.1 | 3.2% | 713, 1148, 493 | 21.0 | 3.8% | 13 |
| 212 | VEGFA | 1428 | 274, 494 | 18.5 | 2.6% | 714, 931, 494 | 32.1 | 5.8% | 714, 1149, 494 | 46.7 | 9.4% | 12 |
| 213 | VEGFA | 1603 | 275, 495 | 14.6 | 2.1% | 715, 932, 495 | 36.6 | 17.5% | 715, 1150, 495 | 65.6 | 6.9% | 13 |
| 214 | VEGFA | 1685 | 276, 496 | 17.1 | 1.3% | 716, 933, 496 | 20.2 | 5.5% | 716, 1151, 496 | 23.4 | 3.8% | 13 |
| 215 | VEGFA | 1792 | 277, 497 | 17.0 | 1.8% | 717, 934, 497 | 21.2 | 3.2% | 717, 1152, 497 | 39.5 | 6.3% | 12 |
| 216 | VEGFA | 2100 | 278, 498 | 116.9 | 11.5% | 718, 935, 498 | 103.6 | 7.5% | 718, 1153, 498 | 101.5 | 12.9% | 12 |
| 217 | VEGFA | 2102 | 279, 499 | 116.3 | 9.1% | 719, 936, 499 | 110.2 | 9.3% | 719, 1154, 499 | 105.0 | 8.0% | 12 |
| 218 | VEGFA | 2196 | 280, 500 | 24.2 | 2.7% | 720, 937, 500 | 26.6 | 3.1% | 720, 1155, 500 | 43.5 | 3.5% | 12 |
| 219 | VEGFA | 2261 | 281, 501 | 15.6 | 2.2% | 721, 938, 501 | 44.2 | 6.2% | 721, 1156, 501 | 109.0 | 9.8% | 12 |
| 220 | VEGFA | 2292 | 282, 502 | 48.4 | 4.3% | 722, 939, 502 | 45.1 | 7.2% | 722, 1157, 502 | 80.7 | 6.7% | 15 |

*All samples were normalized to the respective dsRNA QNeg (Qiagen) negative control samples run in the same experiment. That is, QNeg values were set as 100% active (i.e., no knockdown), with 95% confidence intervals (CI) ranging from 6.3-22.5%. As a positive control, an siRNA specific for rLuc was used, which samples showed on average expression levels that varied from 1.2% to 16.8% (i.e., about 83% to about 99% knockdown activity and a 95% CI ranging from 0.3% to 13.7%).
†“Pos” refers to the position on the target gene mRNA message that aligns with the 5′-end of the dsRNA sense strand. The mRNA numbering is based on the GenBank accession numbers as described herein.
‡The SEQ ID NOS. are provided in the following order: (1) Dicer: sense strand, antisense strand; (2) Nicked: 5′-sense strand fragment, 3′-sense strand fragment, and antisense strand; and (3) Gapped: 5′-sense strand fragment, 3′-sense strand fragment, and antisense strand. The Dicer dsRNA has two strands, while ndsRNA and gdsRNA have three strands each. The nicked or gapped sense strand fragments have three locked nucleic acids each.
\hat{ }“Length 5′-S” refers to the length of the 5′-sense strand fragment of the nicked or gapped mdRNA, which indicates the position of the nick (e.g., 10 means the nick is between position 10 and 11, so the 5′sense strand fragment is 10 nucleotides long and the 3′-sense strand fragment is 15 nucelotides long) or one nucleotide gap (e.g., 10 means the missing nucleotide is number 11, so the 5′sense strand fragment is 10 nucleotides long and the 3′-sense strand fragment is 14 nucelotides long).

### Example 2

### Knockdown of β-Galactosidase Activity by Gapped dsRNA Dicer Substrate

The activity of a Dicer substrate dsRNA containing a gap in the double-stranded structure in silencing LacZ mRNA as compared to the normal Dicer substrate dsRNA (i.e., not having a gap) was examined.

Nucleotide Sequences of dsRNA and mdRNA Targeting LacZ mRNA

The nucleic acid sequence of the one or more sense strands, and the antisense strand of the dsRNA and gapped dsRNA (also referred to herein as a meroduplex or mdRNA) are shown below and were synthesized using standard techniques. The RISC activator LacZ dsRNA comprises a 21 nucleotide sense strand and a 21 nucleotide antisense strand, which can anneal to form a double-stranded region of 19 base pairs with a two deoxythymidine overhang on each strand (referred to as 21/21 dsRNA).

LacZ dsRNA (21/21)—RISC Activator

| | |
|---|---|
| Sense | |
| 5′-CUACACAAAUCAGCGAUUUdTdT-3′ | (SEQ ID NO: 1) |
| Antisense | |
| 3′-dTdTGAUGUGUUUAGUCGCUAAA-5′ | (SEQ ID NO: 2) |

The Dicer substrate LacZ dsRNA comprises a 25 nucleotide sense strand and a 27 nucleotide antisense strand, which can anneal to form a double-stranded region of 25 base pairs with one blunt end and a cytidine and uridine overhang on the other end (referred to as 25/27 dsRNA).

LacZ dsRNA (25/27)—Dicer Substrate

| | |
|---|---|
| Sense | |
| 5′-CUACACAAAUCAGCGAUUUCCAUdGdT-3′ | (SEQ ID NO: 3) |
| Antisense | |
| 3′-CUGAUGUGUUUAGUCGCUAAAGGUA C A -5′ | (SEQ ID NO: 4) |

The LacZ mdRNA comprises two sense strands of 13 nucleotides (5′-portion) and 11 nucleotides (3′-portion) and a 27 nucleotide antisense strand, which three strands can anneal to form two double-stranded regions of 13 and 11 base pairs separated by a single nucleotide gap (referred to as a 13, 11/27 mdRNA). The 5′-end of the 11 nucleotide sense strand fragment may be optionally phosphorylated. The “*” indicates a gap—in this case, a single nucleotide gap (i.e., a cytidine is missing).
LacZ mdRNA (13, 11/27)—Dicer Substrate

(SEQ ID NOS: 5, 6)

| | |
|---|---|
| Sense | 5′-CUACACAAAUCAG*GAUUUCCAUdGdT-3′ |

(SEQ ID NO: 4)

| | |
|---|---|
| Antisense | 3′-CUGAUGUGUUUAGUCGCUAAAGGUA C A-5′ |

Each of the LacZ dsRNA or mdRNA was used to transfect 9lacZ/R cells.

### Transfection

Six well collagen-coated plates were seeded with 5×10⁵ 9lacZ/R cells/well in a 2 ml volume per well, and incubated overnight at 37° C./5% CO₂ in DMEM/high glucose media. Preparation for transfection: 250 μl of OPTIMEM media without serum was mixed with 5 μl of 20 pmol/μl dsRNA and 5 μl of HIPERFECT transfection solution (Qiagen) was mixed with another 250 μl OPTIMEM media. After both mixtures were allowed to equilibrate for 5 minutes, the RNA and transfection solutions were combined and left at room temperature for 20 minutes to form transfection complexes. The final concentration of HIPERFECT was 50 μM, and the dsRNAs were tested at 0.05 nM, 0.1 nM, 0.2 nM, 0.5 nM, 1 nM, 2 nM, 5 nM, and 10 nM, while the mdRNA was tested at 0.2 nM, 0.5 nM, 1 nM, 2 nM, 5 nM, 10 nM, 20 nM, and 50 nM. Complete media was removed, the cells were washed with incomplete OPTIMEM, and then 500 μl transfection mixture was applied to the cells, which were incubated with gentle shaking at 37° C. for 4 hours. After transfecting, the transfection media was removed, cells were washed once with complete DMEM/high glucose media, fresh media added, and the cells were then incubated for 48 hours at 37° C., 5% CO₂.

### β-Galactosidase Assay

Transfected cells were washed with PBS, and then detached with 0.5 ml trypsin/EDTA. The detached cells were suspended in 1 ml complete DMEM/high glucose and transferred to a clean tube. The cells were harvested by centrifugation at 250×g for 5 minutes, and then resuspended in 50 μl 1× lysis buffer at 4° C. The lysed cells were subjected to two freeze-thaw cycles on dry ice and a 37° C. water bath. The lysed samples were centrifuged for 5 minutes at 4° C. and the supernatant was recovered. For each sample, 1.5 μl and 10 μl of lysate was transferred to a clean tube and sterile water added to a final volume of 30 μl followed by the addition of 70 μl o-nitrophenyl-β-D-galactopyranose (ONPG) and 200 μl 1× cleavage buffer with β-mercaptoethanol. The samples were mixed briefly, incubated for 30 minutes at 37° C., and then 500 μl stop buffer was added (final volume 800 μl). β-Galactosidase activity for each sample was measured in disposable cuvettes at 420 nm. Protein concentration was determined by the BCA (bicinchoninic acid) method. For the purpose of the instant example, the level of measured LacZ activity was correlated with the quantity of LacZ transcript within 9L/LacZ cells. Thus, a reduction in β-galactosidase activity after dsRNA transfection, absent a negative impact on cell viability, was attributed to a reduction in the quantity of LacZ transcripts resulting from targeted degradation mediated by the LacZ dsRNA.

### Results

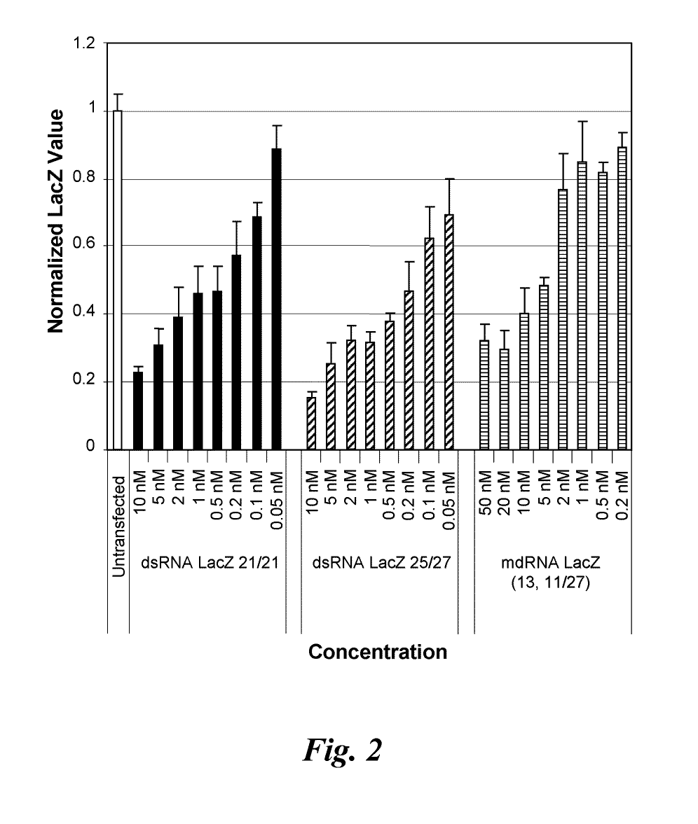

Knockdown activity in transfected and untransfected cells was normalized to a Qneg control dsRNA and presented as a normalized value of the Qneg control (i.e., Qneg represented 100% or “normal” gene expression levels). Both the lacZ RISC activator and Dicer substrate dsRNAs molecule showed good knockdown of β-galactosidase activity at concentration as low as 0.1 nM (FIG. 2), while the Dicer substrate antisense strand alone (single stranded 27mer) had no silencing effect. A gapped mdRNA showed good knockdown although somewhat lower than that of intact RISC activator and Dicer substrate dsRNAs (FIG. 2). The presence of the gapmer cytidine (i.e., the missing nucleotide) at various concentrations (0.1 μM to 50 μM) had no effect on the activity of the mdRNA (data not shown). None of the dsRNA or mdRNA solutions showed any detectable toxicity in the transfected 9L/LacZ cells. The IC₅₀ of the lacZ mdRNA was calculated to be 3.74 nM, which is about 10 fold lower than what had been previously measured for lacZ dsRNA 21/21 (data not shown). These results show that a meroduplex (gapped dsRNA) is capable of inducing gene silencing.

### Example 3

### Knockdown of Influenza Gene Expression by Nicked dsRNA

The activity of a nicked dsRNA (21/21) in silencing influenza gene expression as compared to a normal dsRNA (i.e., not having a nick) was examined.

Nucleotide Sequences of dsRNA and mdRNA Targeting Influenza mRNA

The dsRNA and nicked dsRNA (another form of meroduplex, referred to herein as ndsRNA) are shown below and were synthesized using standard techniques. The RISC activator influenza G1498 dsRNA comprises a 21 nucleotide sense strand and a 21 nucleotide antisense strand, which can anneal to form a double-stranded region of 19 base pairs with a two deoxythymidine overhang on each strand.

G1498-wt dsRNA (21/21)

| | |
|---|---|
| Sense | |
| 5′-GGAUCUUAUUUCUUCGGAGdTdT-3′ | (SEQ ID NO: 7) |
| Antisense | |
| 3′-dTdTCCUAGAAUAAAGAAGCCUC-5′ | (SEQ ID NO :8) |

The RISC activator influenza G1498 dsRNA was nicked on the sense strand after nucleotide 11 to produce a ndsRNA having two sense strands of 11 nucleotides (5′-portion, italic) and 10 nucleotides (3′-portion) and a 21 nucleotide antisense strand, which three strands can anneal to form two double-stranded regions of 11 (shown in italics) and 10 base pairs separated by a one nucleotide gap (which may be referred to as G1498 11, 10/21 ndsRNA-wt). The 5′-end of the 10 nucleotide sense strand fragment may be optionally phosphorylated, as depicted by a “p” preceding the nucleotide (e.g., pC).

G1498 ndsRNA-wt (11, 10/21)

| | |
|---|---|
| Sense | |
| 5′-GGAUCUUAUUUCUUCGGAGdTdT-3′ | (SEQ ID NO: 9, 10) |
| Antisense | |
| 3′-dTdTCCUAGAAUAAAGAAGCCUC-5′ | (SEQ ID NO :8) |

G1498 ndsRNA-wt (11, 10/21)

| | |
|---|---|
| Sense | |
| 5′-GGAUCUUAUUUpCUUCGGAGdTdT-3′ | (SEQ ID NOS: 9, 10) |
| Antisense | |
| 3′-dTdTCCUAGAAUAAAGAAGCCUC-5′ | (SEQ ID NO: 8) |

In addition, each of these G1498 dsRNAs were made with each U substituted with a 5-methyluridine (ribothymidine) and are referred to as G1498 dsRNA-rT. Each of the G1498 dsRNA or ndsRNA (meroduplex), with or without the 5-methyluridine substitution, was used to transfect HeLa S3 cells having an influenza target sequence associated with a luciferase gene. Also, the G1498 antisense strand alone or the antisense strand annealed to the 11 nucleotide sense strand portion alone or the 10 nucleotide sense strand portion alone were examined for activity.

### Transfection and Dual Luciferase Assay

The reporter plasmid psiCHECK™-2 (Promega, Madison, Wis.), which constitutively expresses both firefly luc2 (Photinus pyralis) and Renilla (Renilla reniformis, also known as sea pansy) luciferases, was used to clone in a portion of the influenza NP gene downstream of the Renilla translational stop codon that results in a Renilla-influenza NP fusion mRNA. The firefly luciferase in the psiCHECK™-2 vector is used to normalize Renilla luciferase expression and serves as a control for transfection efficiency.

Multi-well plates were seeded with HeLa S3 cells/well in 100 μl Ham's F12 medium and 10% fetal bovine serum, and incubated overnight at 37° C./5% CO₂. The HeLa S3 cells were transfected with the psiCHECK™-influenza plasmid (75 ng) and G1498 dsRNA or ndsRNA (final concentration of 10 nM or 100 nM) formulated in Lipofectamine™ 2000 and OPTIMEM reduced serum medium. The transfection mixture was incubated with the HeLa S3 cells with gentle shaking at 37° C. for about 18 to 20 hours.

After transfecting, firefly luciferase reporter activity was measured first by adding Dual-Glo™ Luciferase Reagent (Promega, Madison, Wis.) for 10 minutes with shaking, and then quantitating the luminescent signal using a VICTOR³™ 1420 Multilabel Counter (PerkinElmer, Waltham, Mass.). After measuring the firefly luminescence, Stop & Glo® Reagent (Promega, Madison, Wis.) was added for 10 minutes with shaking to simultaneously quench the firefly reaction and initiate the Renilla luciferase reaction, and then the Renilla luciferase luminescent signal was quantitated VICTOR³™ 1420 Multilabel Counter (PerkinElmer, Waltham, Mass.).

### Results

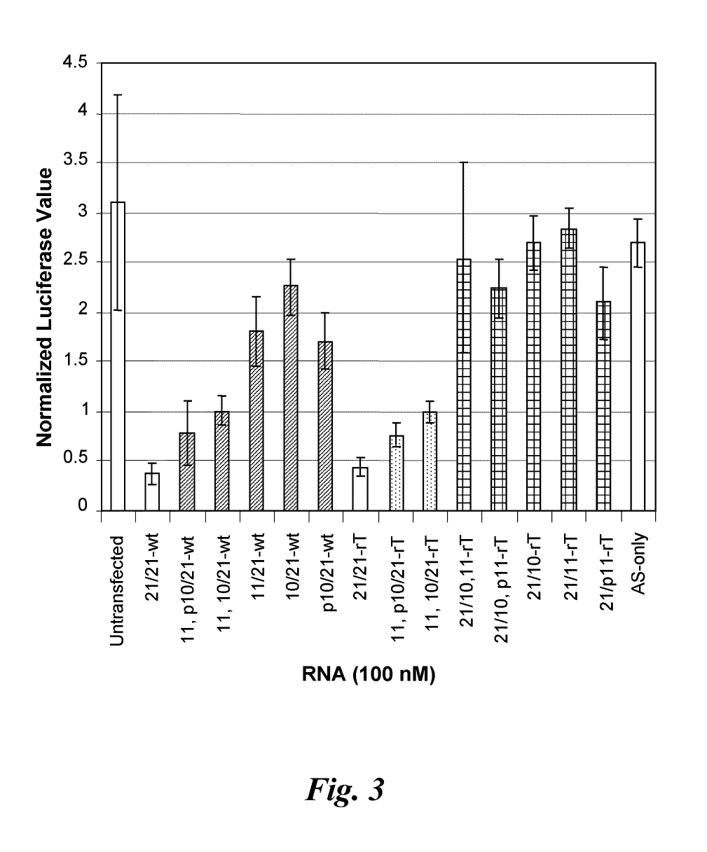

Knockdown activity in transfected and untransfected cells was normalized to a Qneg control dsRNA and presented as a normalized value of the Qneg control (i.e., Qneg represented 100% or “normal” gene expression levels). Thus, a smaller value indicates a greater knockdown effect. The G1498 dsRNA-wt and dsRNA-rT showed similar good knockdown at a 100 nM concentration (FIG. 3). The G1498 ndsRNA-rT, whether phosphorylated or not, showed good knockdown although somewhat lower than the G1498 dsRNA-wt (FIG. 3). Similar results were obtained with dsRNA or ndsRNA at 10 nM (data not shown). None of the G1498 dsRNA or ndsRNA solutions showed any detectable toxicity in HeLa S3 cells at either 10 nM or 100 nM. Even the presence of only half a nicked sense strand (an 11 nucleotide or 10 nucleotide strand alone) with a G1498 antisense strand showed some detectable activity. These results show that a nicked-type meroduplex dsRNA molecule is unexpectedly capable of promoting gene silencing.

### Example 4

### Knockdown Activity of Nicked mdRNA

In this example, the activity of a dicer substrate LacZ dsRNA of Example 1 having a sense strand with a nick at various positions was examined. In addition, a dideoxy nucleotide (i.e., ddG) was incorporated at the 5′-end of the 3′-most strand of a sense sequence having a nick or a single nucleotide gap to determine whether the in vivo ligation of the nicked sense strand is “rescuing” activity. The ddG is not a substrate for ligation. Also examined was the influenza dicer substrate dsRNA of Example 7 having a sense strand with a nick at one of positions 8 to 14. The “p” designation indicates that the 5′-end of the 3′-most strand of the nicked sense influenza sequence was phosphorylated. The “L” designation indicates that the G at position 2 of the 5′-most strand of the nicked sense influenza sequence was substituted for a locked nucleic acid G. The Qneg is a negative control dsRNA.

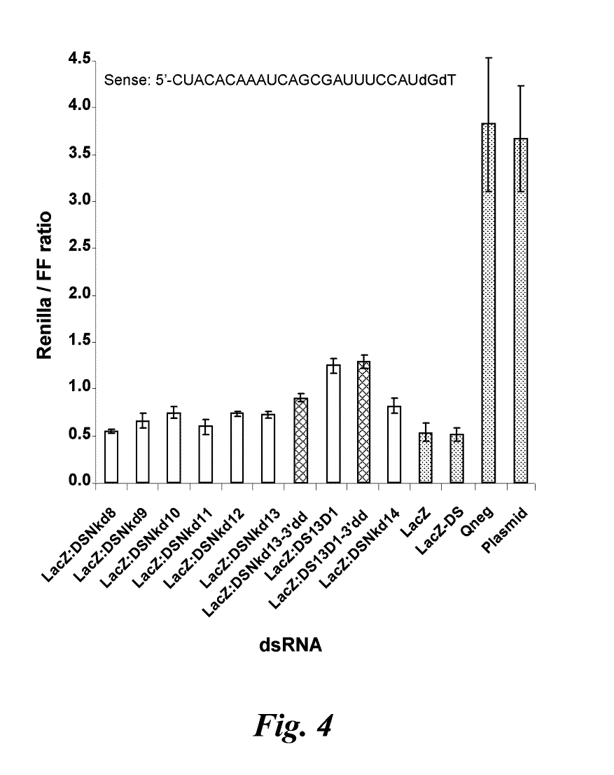

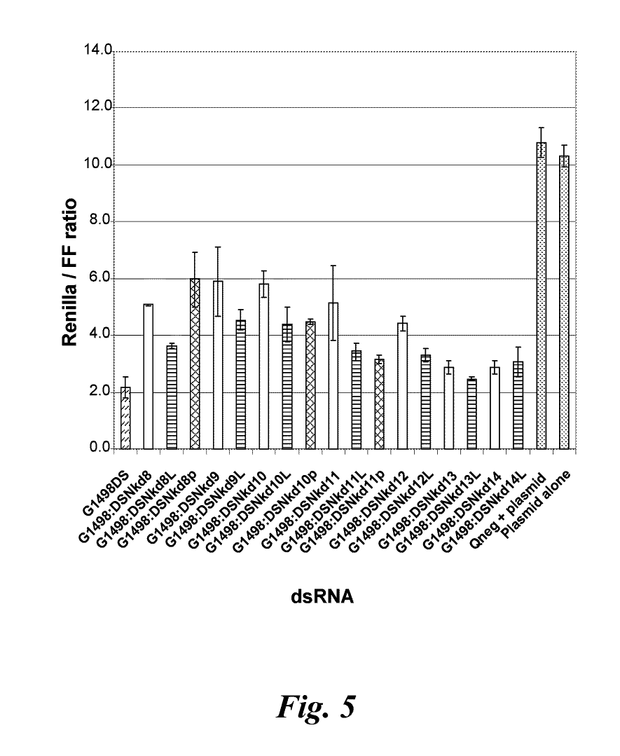

The dual fluorescence assay of Example 3 was used to measure knockdown activity with 5 nM of the LacZ sequences and 0.5 nM of the influenza sequences. The lacZ dicer substrate (25/27, LacZ-DS) and lacZ RISC activator (21/21, LacZ) are equally active, and the LacZ-DS can be nicked in any position between 8 and 14 without affecting activity (FIG. 3). In addition, the inclusion of a ddG on the 5′-end of the 3′-most LacZ sense sequence having a nick (LacZ:DSNkd13-3′dd) or a one nucleotide gap (LacZ:DSNkd13D1-3′dd) was essentially as active as the unsubstituted sequence (FIG. 4). The influenza dicer substrate (G1498DS) nicked at any one of positions 8 to 14 was also highly active (FIG. 5). Phosphorylation of the 5′-end of the 3′-most strand of the nicked sense influenza sequence had essentially no effect on activity, but addition of a locked nucleic acid appears to improve activity.

### Example 5

### Mean Inhibitory Concentration of mdRNA

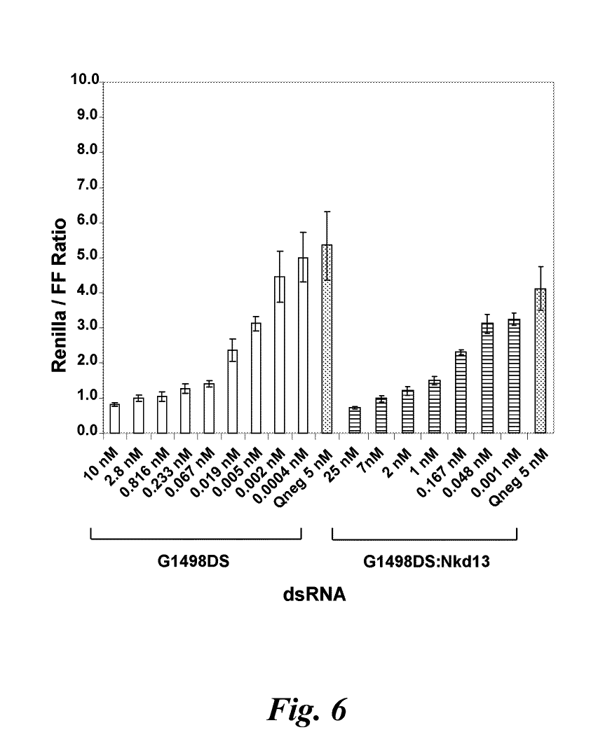

In this example, a dose response assay was performed to measure the mean inhibitory concentration (IC₅₀) of the influenza dicer substrate dsRNA of Example 8 having a sense strand with a nick at position 12, 13, or 14, including or not a locked nucleic acid. The dual luciferase assay of Example 2 was used. The influenza dicer substrate dsRNA (G1498DS) was tested at 0.0004 nM, 0.002 nM, 0.005 nM, 0.019 nM, 0.067 nM, 0.233 nM, 0.816 nM, 2.8 nM, and 10 nM, while the mdRNA with a nick at position 13 (G1498DS:Nkd13) was tested at 0.001 nM, 0.048 nM, 0.167 nM, 1 nM, 2 nM, 7 nM, and 25 nM (see FIG. 6). Also tested were RISC activator molecules (21/21) with or without a nick at various positions (including G1498DS:Nkd11, G1498DS:Nkd12, and G1498DS:Nkd14), each of the nicked versions with a locked nucleic acid as described above (data not shown). The Qneg is a negative control dsRNA.

The IC₅₀ of the RISC activator G1498 was calculated to be about 22 pM, while the dicer substrate G1498DS IC₅₀ was calculated to be about 6 pM. The IC₅₀ of RISC and Dicer mdRNAs range from about 200 pM to about 15 nM. The inclusion of a single locked nucleic acid reduced the IC₅₀ of Dicer mdRNAs by up 4 fold (data not shown). These results show that a meroduplex dsRNA having a nick or gap in any position is capable of inducing gene silencing.

### Example 6

### Knockdown Activity of Gapped mdRNA

The activity of an influenza dicer substrate dsRNA having a sense strand with a gap of differing sizes and positions was examined. The influenza dicer substrate dsRNA of Example 8 was generated with a sense strand having a gap of 0 to 6 nucleotides at position 8, a gap of 4 nucleotides at position 9, a gap of 3 nucleotides at position 10, a gap of 2 nucleotides at position 11, and a gap of 1 nucleotide at position 12 (see Table 2). The Qneg is a negative control dsRNA. Each of the mdRNAs was tested at a concentration of 5 nM (data not shown) and 10 nM. The mdRNAs have the following antisense strand 5′-CAUUGUCUCCGAAGAAAUAAGAUCCUU (SEQ ID NO:11), and nicked or gapped sense strands as shown in Table 2.

TABLE 2

| | | | | | |
|---|---|---|---|---|---|
| | 5′ Sense* | | Gap | Gap | % |
| mdRNA | (SEQ ID NO.) | 3′ Sense (SEQ ID NO.) | Pos | Size | KD^{†} |

| | | | | | |
|---|---|---|---|---|---|
| G1498: DSNkd8 | GGAUCUUA (12) | UUUCUUCGGAGACAAdTdG (13) | 8 | 0 | 67.8 |
| G1498: DSNkd8D1 | GGAUCUUA (12) | UUCUUCGGAGACAAdTdG (14) | 8 | 1 | 60.9 |
| G1498: DSNkd8D2 | GGAUCUUA (12) | UCUUCGGAGACAAdTdG (15) | 8 | 2 | 48.2 |
| G1498: DSNkd8D3 | GGAUCUUA (12) | CUUCGGAGACAAdTdG (16) | 8 | 3 | 44.1 |
| G1498: DSNkd8D4 | GGAUCUUA (12) | UUCGGAGACAAdTdG (17) | 8 | 4 | 30.8 |
| G1498: DSNkd8D5 | GGAUCUUA (12) | UCGGAGACAAdTdG (18) | 8 | 5 | 10.8 |
| G1498: DSNkd8D6 | GGAUCUUA (12) | CGGAGACAAdTdG (19) | 8 | 6 | 17.9 |
| G1498: DSNkd9D4 | GGAUCUUAU (20) | UCGGAGACAAdTdG (18) | 9 | 4 | 38.9 |
| G1498: DSNkd10D3 | GGAUCUUAUU (21) | UCGGAGACAAdTdG (18) | 10 | 3 | 38.4 |
| G1498: DSNkd11D2 | GGAUCUUAUUU (22) | UCGGAGACAAdTdG (18) | 11 | 2 | 46.2 |
| G1498: DSNkd12D1 | GGAUCUUAUUUC (23) | UCGGAGACAAdTdG (18) | 12 | 1 | 49.6 |
| Plasmid | — | — | — | — | 5.3 |

*G indicates a locked nucleic acid G in the 5′ sense strand.
^{†}KD means percent knockdown activity.

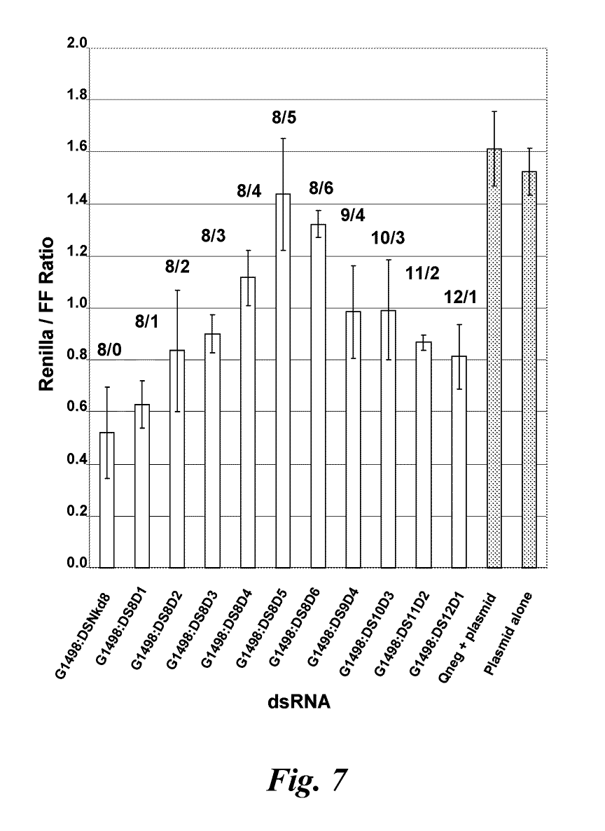

The dual fluorescence assay of Example 2 was used to measure knockdown activity. Similar results were obtained at both the 5 nM and 10 nM concentrations. These data show that an mdRNA having a gap of up to 6 nucleotides still has activity, although having four or fewer missing nucleotides shows the best activity (see, also, FIG. 7). Thus, mdRNA having various sizes gaps that are in various different positions have knockdown activity.

To examine the general applicability of a sequence having a sense strand with a gap of differing sizes and positions, a different dsRNA sequence was tested. The lacZ RISC dsRNA of Example 1 was generated with a sense strand having a gap of 0 to 6 nucleotides at position 8, a gap of 5 nucleotides at position 9, a gap of 4 nucleotides at position 10, a gap of 3 nucleotides at position 11, a gap of 2 nucleotides at position 12, a gap of 1 nucleotide at position 12, and a nick (gap of 0) at position 14 (see Table 3). The Qneg is a negative control dsRNA. Each of the mdRNAs was tested at a concentration of 5 nM (data not shown) and 25 nM. The lacZ mdRNAs have the following antisense strand 5′-AAAUCGCUGAUUUGUGUAGdTdTUAAA (SEQ ID NO:2) and nicked or gapped sense strands as shown in Table 3.

TABLE 3

| | | | | |
|---|---|---|---|---|
| | 5′ Sense* | | Gap | Gap |
| mdRNA | (SEQ ID NO.) | 3′ Sense* (SEQ ID NO.) | Pos | Size |

| | | | | |
|---|---|---|---|---|
| LacZ: Nkd8 | CUACACAA (24) | AUCAGCGAUUUdTdT (25) | 8 | 0 |
| LacZ: Nkd8D1 | CUACACAA (24) | UCAGCGAUUUdTdT (26) | 8 | 1 |
| LacZ Nkd8D2 | CUACACAA (24) | CAGCGAUUUdTdT (27) | 8 | 2 |
| LacZ Nkd8D3 | CUACACAA (24) | AGCGAUUUdTdT (28) | 8 | 3 |
| LacZ Nkd8D4 | CUACACAA (24) | GCGAUUUdTdT (29) | 8 | 4 |
| LacZ: Nkd8D5 | CUACACAA (24) | CGAUUUdTdT (30) | 8 | 5 |
| LacZ: Nkd8D6 | CUACACAA (24) | GAUUUdTdT (31) | 8 | 6 |
| LacZ: Nkd9D5 | CUACACAAA (32) | GAUUUdTdT (31) | 9 | 5 |
| LacZ: Nkd10D4 | CUACACAAAU (33) | GAUUUdTdT (31) | 10 | 4 |
| LacZ: Nkd11D3 | CUACACAAAUC (34) | GAUUUdTdT (31) | 11 | 3 |
| LacZ: Nkd12D2 | CUACACAAAUCA (35) | GAUUUdTdT (31) | 12 | 2 |
| LacZ: Nkd13D1 | CUACACAAAUCAG (36) | GAUUUdTdT (31) | 13 | 1 |
| LacZ: Nkd14 | CUACACAAAUCAGC (37) | GAUUUdTdT (31) | 14 | 0 |

*A indicates a locked nucleic acid A in each sense strand.

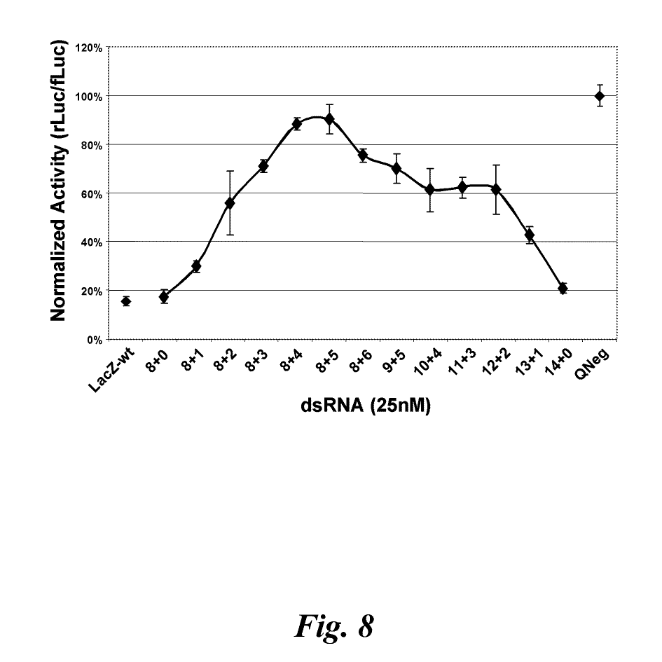

The dual fluorescence assay of Example 3 was used to measure knockdown activity. FIG. 8 shows that an mdRNA having a gap of up to 6 nucleotides has substantial activity and the position of the gap may affect the potency of knockdown. Thus, mdRNA having various sizes gaps that are in various different positions and in different mdRNA sequences have knockdown activity.

### Example 7

### Knockdown Activity of Substituted mdRNA

The activity of an influenza dsRNA RISC sequences having a nicked sense strand and the sense strands having locked nucleic acid substitutions were examined. The influenza RISC sequence G1498 of Example 3 was generated with a sense strand having a nick at positions 8 to 14 counting from the 5′-end. Each sense strand was substituted with one or two locked nucleic acids as shown in Table 4. The Qneg and Plasmid are negative controls. Each of the mdRNAs was tested at a concentration of 5 nM. The antisense strand used was 5′-CUCCGAAGAAAUAAGAUCCdTdT (SEQ ID NO:8).

TABLE 4

| | | | | |
|---|---|---|---|---|
| | | | Nick | % |
| mdRNA | 5′ Sense* (SEQ ID NO.) | 3′ Sense* (SEQ ID NO.) | Pos | KD |

| | | | | |
|---|---|---|---|---|
| G1498-wt | GGAUCUUAUUUCUUCGGAGdTdT (7) | — | — | 85.8 |
| G1498-L | GGAUCUUAUUUCUUCGGAGdTdT (61) | — | — | 86.8 |
| G1498: Nkd8-1 | GGAUCUUA (12) | UUUCUUCGGAGdTdT (47) | 8 | 36.0 |
| G1498: Nkd8-2 | GGAUCUUA (40) | UUUCUUCGGAGdTdT (54) | 8 | 66.2 |
| G1498: Nkd9-1 | GGAUCUUAU (20) | UUCUUCGGAGdTdT (48) | 9 | 60.9 |
| G1498: Nkd9-2 | GGAUCUUAU (41) | UUCUUCGGAGdTdT (55) | 9 | 64.4 |
| G1498: Nkd10-1 | GGAUCUUAUU (21) | UCUUCGGAGdTdT (49) | 10 | 58.2 |
| G1498: Nkd10-2 | GGAUCUUAUU (42) | UCUUCGGAGdTdT (56) | 10 | 68.5 |
| G1498: Nkd11-1 | GGAUCUUAUUU (22) | CUUCGGAGdTdT (50) | 11 | 75.9 |
| G1498: Nkd11-2 | GGAUCUUAUUU (43) | CUUCGGAGdTdT (57) | 11 | 67.1 |
| G1498: Nkd12-1 | GGAUCUUAUUUC (23) | UUCGGAGdTdT (51) | 12 | 59.9 |
| G1498: Nkd12-2 | GGAUCUUAUUUC (44) | UUCGGAGdTdT (58) | 12 | 72.8 |
| G1498: Nkd13-1 | GGAUCUUAUUUCU (38) | UCGGAGdTdT (52) | 13 | 37.1 |
| G1498: Nkd13-2 | GGAUCUUAUUUCU (45) | UCGGAGdTdT (59) | 13 | 74.3 |
| G1498: Nkd14-1 | GGAUCUUAUUUCUU (39) | CGGAGdTdT (53) | 14 | 29.0 |
| G1498: Nkd14-2 | GGAUCUUAUUUCUU (46) | CGGAGdTdT (60) | 14 | 60.2 |
| Qneg | — | — | — | 0 |
| Plasmid | — | — | — | 3.6 |

*Nucleotides that are bold and underlined are locked nucleic acids.

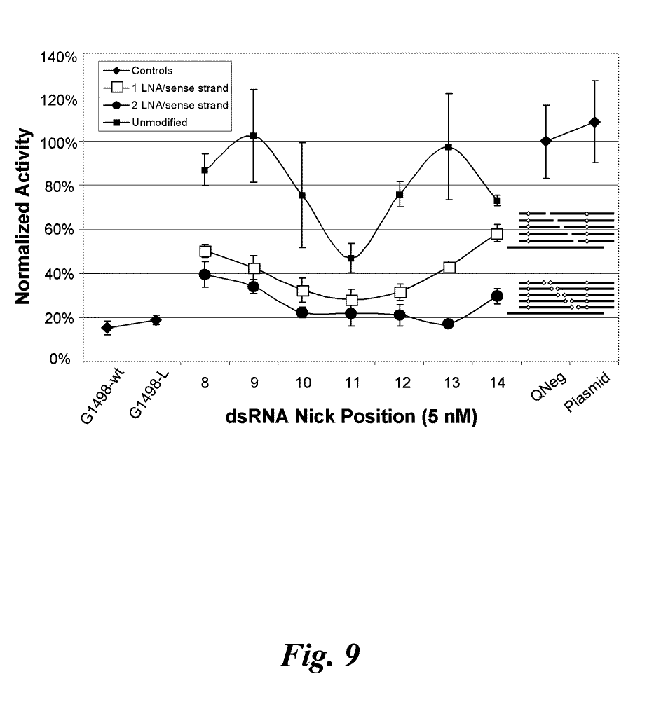

The dual fluorescence assay of Example 3 was used to measure knockdown activity. These data show that increasing the number of locked nucleic acid substitutions tends to increase activity of an mdRNA having a nick at any of a number of positions. The single locked nucleic acid per sense strand appears to be most active when the nick is at position 11 (see FIG. 9). But, multiple locked nucleic acids on each sense strand make mdRNA having a nick at any position as active as the most optimal nick position with a single substitution (i.e., position 11) (FIG. 9). Thus, mdRNA having duplex stabilizing modifications make mdRNA essentially equally active regardless of the nick position.

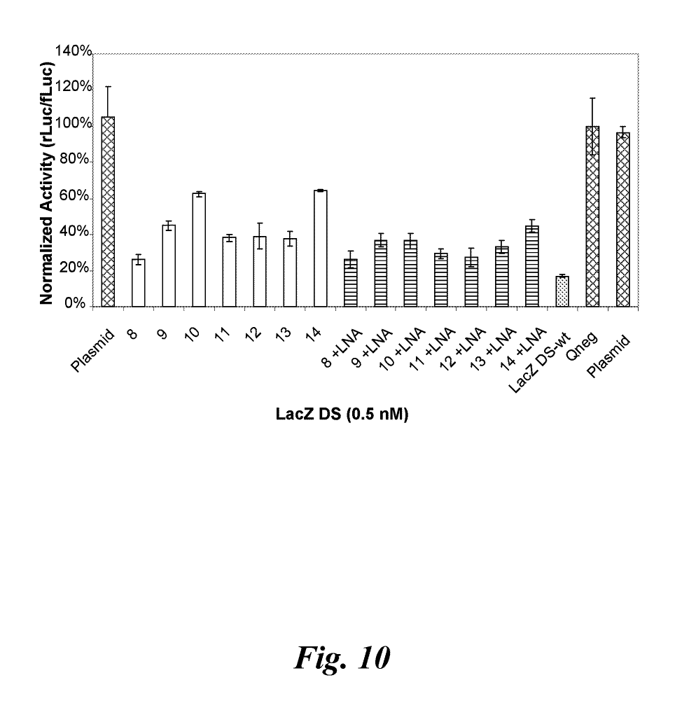

Similar results were observed when locked nucleic acid substitutions were made in the LacZ dicer substrate mdRNA of Example 2 (SEQ ID NOS:3 and 4). The lacZ dicer was nicked at positions 8 to 14, and a duplicate set of nicked LacZ dicer molecules were made with the exception that the A at position 3 (from the 5′-end) of the 5′ sense strand was substituted for a locked nucleic acid A (LNA-A). As is evident from FIG. 10, most of the nicked lacZ dicer molecules containing LNA-A were as potent in knockdown activity as the unsubstituted lacZ dicer.

### Example 8

### mdRNA Knockdown of Influenza Virus Titer

The activity of a dicer substrate nicked dsRNA in reducing influenza virus titer as compared to a wild-type dsRNA (i.e., not having a nick) was examined. The influenza dicer substrate sequence (25/27) is as follows:

| | |
|---|---|
| Sense | |
| 5′-GGAUCUUAUUUCUUCGGAGACAAdTdG | (SEQ ID NO: 62) |
| Antisense | |
| 5′-CAUUGUCUCCGAAGAAAUAAGAUCCUU | (SEQ ID NO: 11) |

The mdRNA sequences have a nicked sense strand after position 12, 13, and 14, respectively, as counted from the 5′-end, and the G at position 2 is substituted with locked nucleic acid G.

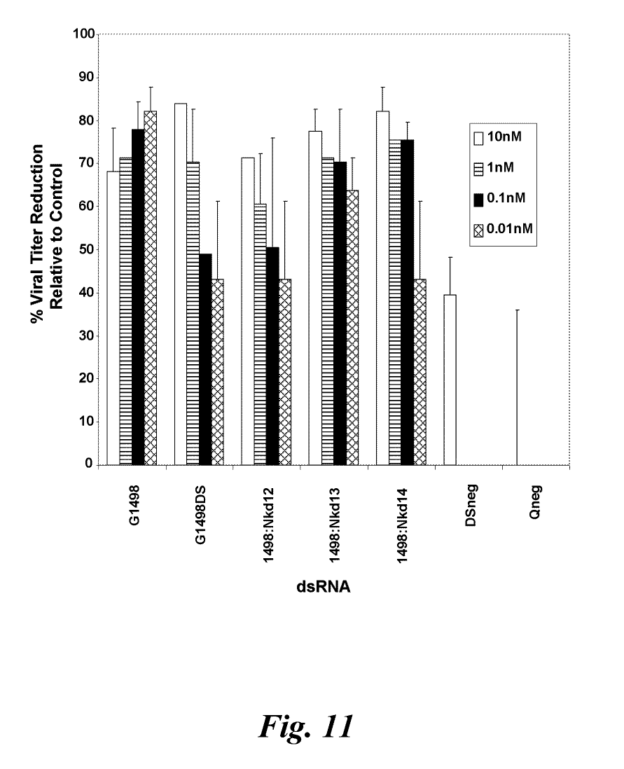

For the viral infectivity assay, Vero cells were seeded at 6.5×10⁴ cells/well the day before transfection in 500 μl A 10% FBS/DMEM media per well. Samples of 100, 10, 1, 0.1, and 0.01 nM stock of each dsRNA were complexed with 1.0 μl (1 mg/ml stock) of Lipofectamine™ 2000 (Invitrogen, Carlsbad, Calif.) and incubated for 20 minutes at room temperature in 150 μl OPTIMEM (total volume) (Gibco, Carlsbad, Calif.). Vero cells were washed with OPTIMEM, and 150 μl of the transfection complex in OPTIMEM was then added to each well containing 150 μl of OPTIMEM media. Triplicate wells were tested for each condition. An additional control well with no transfection condition was prepared. Three hours post transfection, the media was removed. Each well was washed once with 200 μl PBS containing 0.3% BSA and 10 mM HEPES/PS. Cells in each well were infected with WSN strain of influenza virus at an MOI 0.01 in 200 μl of infection media containing 0.3% BSA/10 mM HEPES/PS and 4 μg/ml trypsin. The plate was incubated for 1 hour at 37° C. Unadsorbed virus was washed off with the 200 μl of infection media and discarded, then 400 μl DMEM containing 0.3% BSA/10 mM HEPES/PS and 4 μg/ml trypsin was added to each well. The plate was incubated at 37° C., 5% CO₂ for 48 hours, then 50 μl supernatant from each well was tested in duplicate by TCID₅₀ assays (50% Tissue-Culture Infective Dose, WHO protocol) in MDCK cells and titers were estimated using the Spearman and Karber formula. The results show that these mdRNAs show about a 50% to 60% viral titer knockdown, even at a concentration as low as 10 pM (FIG. 11).

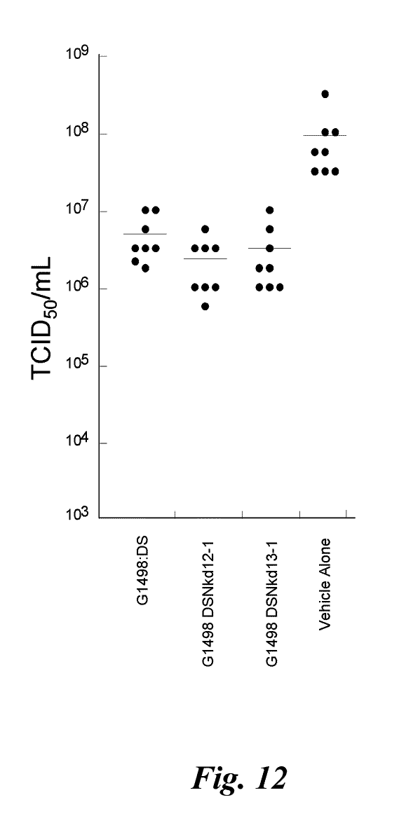

An in vivo influenza mouse model was also used to examine the activity of a dicer substrate nicked dsRNA in reducing influenza virus titer as compared to a wild-type dsRNA (i.e., not having a nick). Female BALB/c mice (age 8-10 weeks with 5-10 mice per group) were dosed intranasally with 120 nmol/kg/day dsRNA (formulated in C12-norArg(NH₃+Cl⁻)—C12/DSPE-PEG2000/DSPC/cholesterol at a ratio of 30:1:20:49) for three consecutive days before intranasal challenge with influenza strain PR8 (20 PFU/mouse). Two days after infection, whole lungs are harvested from each mouse and placed in a solution of PBS/0.3% BSA with antibiotics, homogenize, and measure the viral titer (TCID₅₀). Doses were well tolerated by the mice, indicated by less than 2% body weight reduction in any of the dose groups. The mdRNAs tested exhibit similar, if not slightly greater, virus reduction in vivo as compared to unmodified and unnicked G1498 dicer substrate (see FIG. 12). Hence, mdRNA are active in vivo.

### Example 9

### Effect of mdRNA on Cytokine Induction

The effect of the mdRNA structure on cytokine induction in vivo was examined. Female BALB/c mice (age 7-9 weeks) were dosed intranasally with about 50 μM dsRNA (formulated in C12-norArg(NH₃+Cl—)—C12/DSPE-PEG2000/DSPC/cholesterol at a ratio of 30:1:20:49) or with 605 nmol/kg/day naked dsRNA for three consecutive days. About four hours after the final dose is administered, the mice were sacrificed to collect bronchoalveolar fluid (BALF), and collected blood is processed to serum for evaluation of the cytokine response. Bronchial lavage was performed with 0.5 mL ice-cold 0.3% BSA in saline two times for a total of 1 mL. BALF was spun and supernatants collected and frozen until cytokine analysis. Blood was collected from the vena cava immediately following euthanasia, placed into serum separator tubes, and allowed to clot at room temperature for at least 20 minutes. The samples were processed to serum, aliquoted into Millipore ULTRAFREE 0.22 μm filter tubes, spun at 12,000 rpm, frozen on dry ice, and then stored at −70° C. until analysis. Cytokine analysis of BALF and plasma were performed using the Procarta™ mouse 10-Plex Cytokine Assay Kit (Panomics, Fremont, Calif.) on a Bio-Plex™ array reader. Toxicity parameters were also measured, including body weights, prior to the first dose on day 0 and again on day 3 (just prior to euthanasia). Spleens were harvested and weighed (normalized to final body weight). The results are provided in Table 5.

TABLE 5
In vivo Cytokine Induction by Naked mdRNA

| | | | G1498:Nkd | | G1498:DSNkd | G1498:DSNkd | G1498:DSNkd |
|---|---|---|---|---|---|---|---|
| Cytokine | | G1498 | 11-1 | G1498:DS | 12-1 | 13-1 | 14-1 |
| IL-6 | Conc (pg/mL) | 90.68 | 10.07 | 77.35 | 17.17 | 18.21 | 38.59 |
| | Fold decrease | — | 9 | — | 5 | 4 | 2 |
| IL-12 | Conc (pg/mL) | 661.48 | 20.32 | 1403.61 | 25.07 | 37.70 | 57.02 |
| (p40) | Fold decrease | — | 33 | — | 56 | 37 | 25 |
| TNFα | Conc (pg/mL) | 264.49 | 25.59 | 112.95 | 20.52 | 29.00 | 64.93 |
| | Fold decrease | — | 10 | — | 6 | 4 | 2 |

The mdRNA (RISC or dicer sized) induced cytokines to lesser extent than the intact (i.e., not nicked) parent molecules. The decrease in cytokine induction was greatest when looking at IL-12(p40), the cytokine with consistently the highest levels of induction of the 10 cytokine multiplex assay. For the mdRNA, the decrease in IL-12 (p40) ranges from 25- to 56-fold, while the reduction in either IL-6 or TNFα induction was more modest (the decrease in these two cytokines ranges from 2- to 10-fold). Thus, the mdRNA structure appears to provide an advantage in vivo in that cytokine induction is minimized compared to unmodified dsRNA.

Similar results were obtained with the formulated mdRNA, although the reduction in induction was not as prominent. In addition, the presence or absence of a locked nucleic acid has no effect on cytokine induction. These results are shown in Table 6.

TABLE 6
In vivo Cytokine Induction by Formulated mdRNA

| | | | G1498:Nkd | G1498:Nkd | G1498:DSNkd | G1498:DSNkd |
|---|---|---|---|---|---|---|
| Cytokine | | G1498:DS | 12-1 | 13-1 | 14-1 | 13 |
| IL-6 | Conc (pg/mL) | 29.04 | 52.95 | 10.28 | 7.79 | 44.29 |
| | Fold decrease | — | −1.8 | 3 | 4 | −1.5 |
| IL-12 | Conc (pg/mL) | 298.93 | 604.24 | 136.45 | 126.71 | 551.49 |
| (p40) | Fold decrease | — | 0 | 2 | 2 | 1 |
| TNFα | Conc (pg/mL) | 13.49 | 21.35 | 3.15 | 3.15 | 18.69 |
| | Fold decrease | — | −1.6 | 4 | 4 | 1.4 |

### Example 10

### Survivin siRNA Induce Caspase Activation

Induction of caspase activity and cell death by transfection of Survivin siRNA in a KU-7 bladder cancer cell line was examined.

Both Caspase 3 and 7 are effector caspases that mediate programmed cell death (i.e., apoptosis), which plays an important role in preventing cancer. Therefore, the ability of a drug, for example an siRNA, to induce the activity of caspases in a cancer or pre-cancer cell and consequently induce apoptosis to prevent cancer or treat cancer in a human subject is highly advantageous. In this Example, Survivin siRNA transfected into a bladder cancer cell line induced Caspase 3 and Caspase 7 activity, and further induced cell morphology changes observed via microscopy analysis that were highly indicative of apopotsis. Accordingly, the data indicate that Survivin siRNA may induce apoptosis in bladder cancer cells.

Briefly, KU-7 cells were plated at a density of 7,500 cells/well on a 96-well plate. Twenty-fours later, 25 μL mixture containing 25 nM or 5 nM siRNA and RNAi MAX diluted 1/50 in optiMEM media was added to each well containing 75 μL cell medium with 10% fetal bovine serum. The transfection was performed in triplacate. The transfection mixture was incubated with the cells for 24 hours. Following the incubation, cells were lysed, RNA extracted and qRT-PCR was performed to determine gene expression levels. The Qneg siRNA served as the negative control. Separately, the transfected cells of the duplicate plate were lysed, and CASPASE-GLO reagent was added (PROMEGA) to measure caspase activity per the manufacturer's protocol. Caspase activity was measured with a Wallac Plate reader. Cell viability was measured with the CELLTITER 96 assay kit (PROMEGA) per the manufacturer's protocol.

The sequence specific Survivin siRNA and a negative control siRNA (i.e., a scrambled sequence of a Survivin siRNA) used in this Example are shown below. Knockdown activity in transfected and untransfected cells was normalized to a Qneg control dsRNA (QIAGEN) and presented as a normalized value of the Qneg control (i.e., Qneg represented 100% or “normal” gene expression levels).

Survivin-11 (DX9792):

| | |
|---|---|
| Sense Strand: | |
| 5′-CCAGUGUUUCUUCUGCUUCTT-3′ | (SEQ ID NO: 1896) |
| Antisense Strand: | |
| 5′-GAAGCAGAAGAAACACUGGTT-3′ | (SEQ ID NO: 1897) |

Survivin-11UNA (DX9794):

Sense Strand:

(SEQ ID NO: 1898)

5′-CCAGUGUUUCUUCUGCUUCunaUunaU-3′
Antisense Strand:

(SEQ ID NO: 1899)

5′-GAAGCAGAAGAAACACUGGunaUunaU-3′

Survivin-11UNA-SCR (DX9794)—Negative Control:

Sense Strand:

(SEQ ID NO: 1900)

5′-UCCCGUUCUAGUGUUUCCUunaUunaU-3′
Antisense Strand:

(SEQ ID NO: 1901)

5′-AGGAAACACUAGAACGGGAunaUunaU-3′

Hydroxymethyl substituted monomer(s) in the sequences of the table below are identified as “unaX” where X is the one letter code for the nucleomonomer (e.g., “unaU” indicates that the uracil comprises a hydroxymethyl substituted monomer). In this Example, the Survivin:286UNA siRNA is a double-stranded RNA having a 19 base pair region with two blunt ends and two hydroxymethyl substituted monomers were covalently linked to the 3′-end of both the sense strand and the antisense strand.

The results for the percent survivin gene knockdown, percent cell death induction, and the fold caspase induction are shown below in Table 10. The percent knockdown (% KD) is relative to Qneg siRNA, percent cell death induction (% CD) is relative to untransfected cells; and fold caspase induction (Ca) is also relative to untransfected cells.

TABLE 10

| | siRNA | KU-7 Cells | | |
|---|---|---|---|---|
| siRNA Identifier | Conc. | % KD | % CD | Ca |
| Survivin-11 | 25 nM | 80 | 19 | 7.1 |
| (DX9792) | 5 nM | 85 | 17 | 5.2 |
| Survivin-11UNA | 25 nM | 79 | 23 | 6.6 |
| (DX9794) | 5 nM | 84 | 23 | 5.2 |
| Survivin-11UNA-SCR | 25 nM | 0 | 9 | 1 |
| (DX9794) | 5 nM | 15 | 8 | 1.1 |

The results show that Survivin siRNA reducted target gene expression levels relative to the Qneg siRNA negative control in the KU-7 bladder cancer cell line. Further, Survivin siRNA induced over a five-fold increase in caspase activity relative to untransfected cells. Lastly, these results show that there is a correlation between siRNA mediated knockdown of Survivin gene expression and both cell death induction and caspase activity induction. In other words, upon a reduction in Survivin gene expression mediated by the Survivin siRNA, both caspase activity increases and cell death induction increases.

What is claimed is:

1. An RNA molecule that down regulates the expression of a target mRNA, the RNA molecules comprising a first strand that is complementary to a human target mRNA and a second strand complementary to the first strand, wherein the first strand and second strands can anneal to form a double-stranded region having from 15 base pairs to 40 base pairs.

2. The RNA molecule of claim 1 wherein the first strand is from 19 to 24 nucleotides in length or from 25 to 29 nucleotides in length and is complementary to a human target gene selected from the group consisting of tumor necrosis factor (TNF), vascular endothelial growth factor (VEGF), vascular endothelial growth factor receptor (VEGFR), epidermal growth factor receptor (EGFR), erythroblastic leukemia viral oncogene homolog (ERBB), platelet derived growth factor (PDGF), platelet derived growth factor receptor (PDGFR), breakpoint cluster region (BCR)-abelson murine leukemia viral oncogene homolog (ABL), steroid-5-alpha-reductase, alpha polypeptide 1 (SRD5A1), steroid-5-alpha-reductase, alpha polypeptide 2 (SRD5A2), phosphoinositide-3-kinase, catalytic (PIK3C), mitogen-activated protein kinase (MAPK), p38 MAPK family, hypoxia-inducible factor 1 alpha (HIF1A), protein kinase N3 (PKN3), interleukin 17A (IL17A), interleukin 6 (IL6), interleukin 18 (IL18), tumor necrosis factor (ligand) superfamily member 13b (TNFSF13B), mitogen-activated protein kinase 1 (MAPK1), v-raf-1 murine leukemia viral oncogene homolog 1 (RAF1), v-AKT murine thymoma viral oncogene (AKT), FK506 binding protein 12-rapamycin associated protein 1 (FRAP1), mitogen-activated protein kinase 2 (MAPK2), cyclin-dependent kinase 2 (CDK2), ATP-binding cassette, subfamily B, member 1 (ABCB1), B-cell CLL/lymphoma 2 (BCL2), angiopoietin 2 (ANGPT2), checkpoint kinase 1 homolog (CHEK1), insulin-like growth factor 1 receptor (IGF1R), signal transducer and activator of transcription 3 (STAT3), matrix metalloproteinase (MMP), folate hydrolase (prostate-specific membrane antigen) 1 (FOLH1), v-myc myelocytomatosis viral oncogene homolog (avian) (MYC), telomerase RNA component (TERC), telomerase reverse transcriptase (TERT), protein kinase C, alpha (PRKCA), RAS viral (v-ras) oncogene homolog (RAS), chemokine (C-X-C motif) ligand or receptor (CXC), Wingless-Type MMTV (Murine Mammary Tumor Virus) Integration Site (WNT), toll-like receptor (TLR), Fc fragment of IgE, low affinity II, receptor for (CD23) (FCER2), FOS gene, (FOS, FOSB, FOSL1, OR FOSL2), hydroxysteroid (11-beta) dehydrogenase (HSD11B1), JUN gene (cJUN, JUNB, or JUND), thymidine phosphorylase (TYMP), early growth response (EGR), zeste homolog 2 (EZH2), cyclin D1 (CCND1), Fas (TNF receptor superfamily, member 6) (FAS), proliferating cell nuclear antigen (PCNA), fibroblast growth factor 2 (FGF2), tumor growth factor-beta (TGF-β), tumor growth factor-beta receptor (TGF-βR), tumor-associated calcium signal transducer 1 (TACSTD1), Mucin 1 (MUC1), protein tyrosine phosphatase, non-receptor-11 (Noonan Syndrome 1) (PTPN11), neuregulin 1 (NRG1), membrane metallo-endopeptidase (MME), CD19 molecule (CD19), CD40 molecule, TNF receptor superfamily member 5 (CD40), apolipoprotein B (including Ag(x) antigen) (ApoB), synuclein, alpha (non A4 component of amyloid precursor) (SNCA), silent mating type information regulation 2 homolog (SIRT2), histone deacetylase (HDAC), membrane-spanning 4-domains, subfamily A, member 1 (MS4A1), CD22 molecule (CD22), diacylglycerol o-acyltransferase 1 (DGAT1), diacylglycerol o-acyltransferase 2 (DGAT2), CD3 molecule (CD3), proprotein convertase subtilisin-like kexin type 9 (PCSK9), MET (Mesenchymal epithelial transition factor) (c-Met proto-oncogene), catenin (cadherin-associated protein) (beta-catenin) (CTNNB1), inhition of DNA binding proteins (Inhibition of Differentiation Proteins, Dominant Negative Helix-Loop-Helix Protein) (ID), protein tyrosine phosphatase, non-receptor type 1 (PTPN1), tie-1 (TIE1; tyrosine kinase with immunoglobulin and EGF factor homology domains 1), tek tyrosine kinase (TEK), fibroblast growth factor receptor (FGFR), mitogen-activated protein kinase 3 (MAPK3), survivin (BIRC5), and polo-like kinase family genes (PLK1).

3. The RNA molecule of claim 1 wherein the RNA molecule has at least one blunt end.

4. The RNA molecule of claim 1 wherein the RNA molecule has at least one 3′-overhang.

5. The RNA molecule of claim 1 further comprising at least one acyclic nucleomonomer.

6. The RNA molecule of claim 5 wherein the acyclic nucleomonomer is selected from the group consisting of: 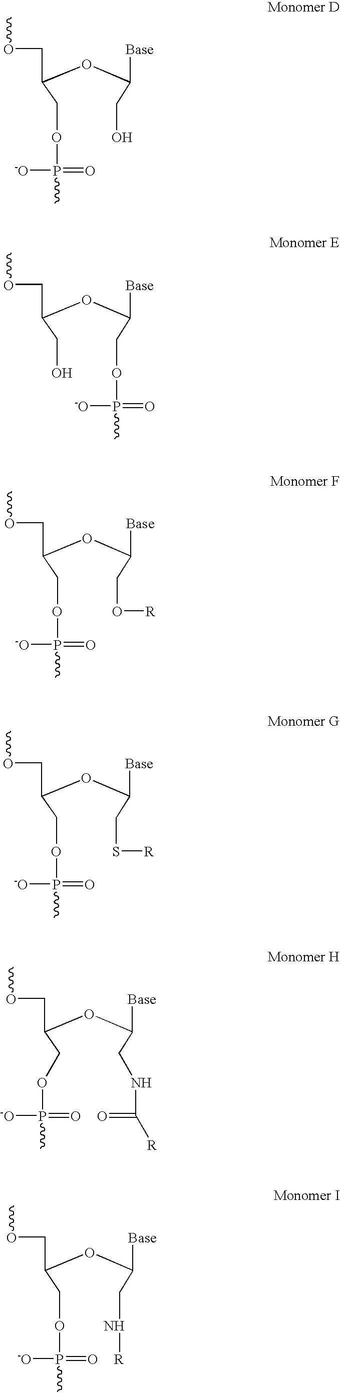 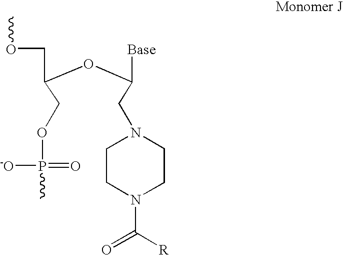
wherein,
R is selected from the group consisting of hydrogen, a methyl group, C(1-10) alkyl, cholesterol, naturally or non-naturally occurring amino acid, sugar, vitamin, fluorophore, polyamine and fatty acid.

7. The RNA molecule of claim 6 wherein at least one acyclic nucleomonomer is linked to the blunt end of the RNA molecule.

8. The RNA molecule of claim 6 at least one acyclic nucleomonomer is in the double-stranded region of the RNA molecule.

9. A method for reducing the expression of a human target gene, comprising administering an RNA molecule of claim 1 to a cell expressing the target gene, wherein the RNA molecule reduces expression of the target gene in the cell.

10. The method according to claim 9 wherein the cell is a human cell.

11. A meroduplex ribonucleic acid (mdRNA) molecule that down regulates the expression of a target mRNA, the mdRNA molecules comprising a first strand of 15 to 40 nucleotides in length that is complementary to a target mRNA and a second strand and a third strand that are each complementary to non-overlapping regions of the first strand, wherein the second strand and third strand can anneal with the first strand to form at least two double-stranded regions spaced apart by a nick or a gap.

12. The mdRNA molecule of claim 11 wherein the first strand is 15 to 25 nucleotides in length or 26 to 40 nucleotides in length.

13. The mdRNA molecule of claim 11 wherein the first strand is from 19 to 24 nucleotides in length or from 25 to 29 nucleotides in length and is complementary to a human target gene selected from the group consisting of tumor necrosis factor (TNF), vascular endothelial growth factor (VEGF), vascular endothelial growth factor receptor (VEGFR), epidermal growth factor receptor (EGFR), erythroblastic leukemia viral oncogene homolog (ERBB), platelet derived growth factor (PDGF), platelet derived growth factor receptor (PDGFR), breakpoint cluster region (BCR)-abelson murine leukemia viral oncogene homolog (ABL), steroid-5-alpha-reductase, alpha polypeptide 1 (SRD5A1), steroid-5-alpha-reductase, alpha polypeptide 2 (SRD5A2), phosphoinositide-3-kinase, catalytic (PIK3C), mitogen-activated protein kinase (MAPK), p38 MAPK family, hypoxia-inducible factor 1 alpha (HIF1A), protein kinase N3 (PKN3), interleukin 17A (IL17A), interleukin 6 (IL6), interleukin 18 (IL18), tumor necrosis factor (ligand) superfamily member 13b (TNFSF13B), mitogen-activated protein kinase 1 (MAPK1), v-raf-1 murine leukemia viral oncogene homolog 1 (RAF1), v-AKT murine thymoma viral oncogene (AKT), FK506 binding protein 12-rapamycin associated protein 1 (FRAP1), mitogen-activated protein kinase 2 (MAPK2), cyclin-dependent kinase 2 (CDK2), ATP-binding cassette, subfamily B, member 1 (ABCB1), B-cell CLL/lymphoma 2 (BCL2), angiopoietin 2 (ANGPT2), checkpoint kinase 1 homolog (CHEK1), insulin-like growth factor 1 receptor (IGF1R), signal transducer and activator of transcription 3 (STAT3), matrix metalloproteinase (MMP), folate hydrolase (prostate-specific membrane antigen) 1 (FOLH1), v-myc myelocytomatosis viral oncogene homolog (avian) (MYC), telomerase RNA component (TERC), telomerase reverse transcriptase (TERT), protein kinase C, alpha (PRKCA), RAS viral (v-ras) oncogene homolog (RAS), chemokine (C-X-C motif) ligand or receptor (CXC), Wingless-Type MMTV (Murine Mammary Tumor Virus) Integration Site (WNT), toll-like receptor (TLR), Fc fragment of IgE, low affinity II, receptor for (CD23) (FCER2), FOS gene, (FOS, FOSB, FOSL1, OR FOSL2), hydroxysteroid (11-beta) dehydrogenase (HSD11B1), JUN gene (cJUN, JUNB, or JUND), thymidine phosphorylase (TYMP), early growth response (EGR), zeste homolog 2 (EZH2), cyclin D1 (CCND1), Fas (TNF receptor superfamily, member 6) (FAS), proliferating cell nuclear antigen (PCNA), fibroblast growth factor 2 (FGF2), tumor growth factor-beta (TGF-β), tumor growth factor-beta receptor (TGF-βR), tumor-associated calcium signal transducer 1 (TACSTD1), Mucin 1 (MUC1), protein tyrosine phosphatase, non-receptor-11 (Noonan Syndrome 1) (PTPN11), neuregulin 1 (NRG1), membrane metallo-endopeptidase (MME), CD19 molecule (CD19), CD40 molecule, TNF receptor superfamily member 5 (CD40), apolipoprotein B (including Ag(x) antigen) (ApoB), synuclein, alpha (non A4 component of amyloid precursor) (SNCA), silent mating type information regulation 2 homolog (SIRT2), histone deacetylase (HDAC), membrane-spanning 4-domains, subfamily A, member 1 (MS4A1), CD22 molecule (CD22), diacylglycerol o-acyltransferase 1 (DGAT1), diacylglycerol o-acyltransferase 2 (DGAT2), CD3 molecule (CD3), proprotein convertase subtilisin-like kexin type 9 (PCSK9), MET (Mesenchymal epithelial transition factor) (c-Met proto-oncogene), catenin (cadherin-associated protein) (beta-catenin) (CTNNB1), inhition of DNA binding proteins (Inhibition of Differentiation Proteins, Dominant Negative Helix-Loop-Helix Protein) (ID), protein tyrosine phosphatase, non-receptor type 1 (PTPN1), tie-1 (TIE1; tyrosine kinase with immunoglobulin and EGF factor homology domains 1), tek tyrosine kinase (TEK), fibroblast growth factor receptor (FGFR), mitogen-activated protein kinase 3 (MAPK3), survivin (BIRC5), and polo-like kinase family genes (PLK1).

14. The mdRNA molecule of claim 11 wherein the gap comprises from 1 to 10 unpaired nucleotides.

15. The mdRNA molecule of claim 11 wherein the mdRNA molecule has at least one blunt end.

16. The mdRNA molecule of claim 11 wherein the mdRNA molecule has at least one 3′-overhang comprising one to four nucleotides that are not part of the gap.

17. The mdRNA molecule of claim 11 further comprising at least on acyclic nucleomonomer.

18. The mdRNA molecule of claim 17 wherein the at least one acyclic nucleomonomer selected from the group consisting of: 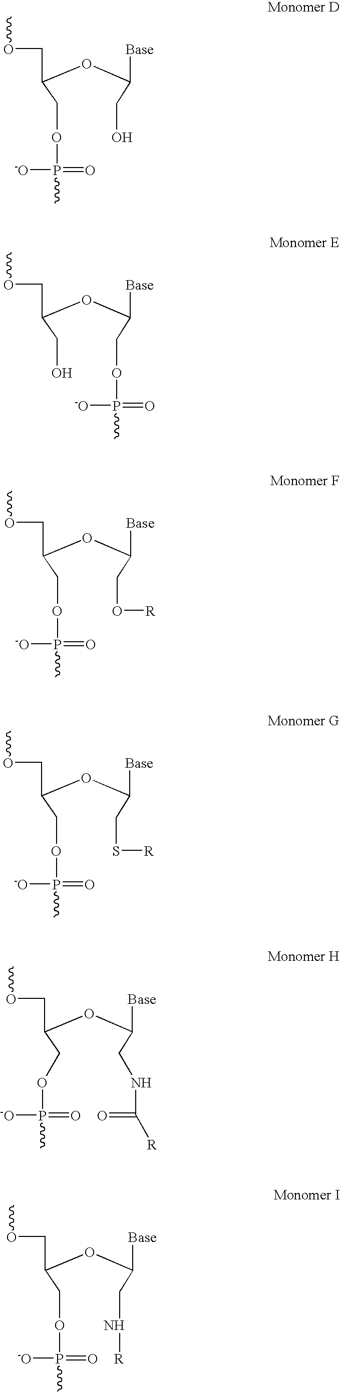 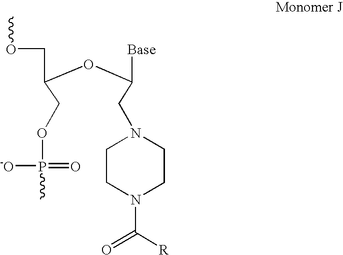
wherein,
R is selected from the group consisting of hydrogen, methyl group, C(1-10) alkyl, cholesterol, naturally or non-naturally occurring amino acid, sugar, vitamin, fluorophore, polyamine and fatty acid.

19. The mdRNA molecule of claim 18 wherein at least one acyclic nucleomonomer is linked to the blunt end of the mdRNA molecule.

20. The mdRNA molecule of claim 18 at least one acyclic nucleomonomer is in one of the double-stranded regions of the mdRNA molecule.

21. A method for reducing the expression of a human target gene, comprising administering an mdRNA molecule of claim 11 to a cell expressing the target gene, wherein the mdRNA molecule reduces expression of the target gene in the cell.

22. The method according to claim 21 wherein the cell is a human cell.
